(19) United States
(12) Patent Application Publication
(10) Pub. No.: US 2013/0190529 A1
(43) Pub. Date: Jul. 25, 2013

(54) Fluorine Radiolabelling Process

(71) Applicants: Veronique Gouveneur, Oxford (GB); Lei Li, Oxford (GB); Yee-Hwee Lim, Oxford (GB); Mickael Huiban, London (GB)

(72) Inventors: Veronique Gouveneur, Oxford (GB); Lei Li, Oxford (GB); Yee-Hwee Lim, Oxford (GB); Mickael Huiban, London (GB)

(21) Appl. No.: 13/806,511

(22) PCT Filed: Jul. 6, 2011
(86) PCT No.: PCT/GB2011/001027

§ 371 Date: Mar. 11, 2013

(30) Foreign Application Priority Data
Jul. 6, 2010 (GB) 1011408.0

(51) Int. Cl. C07C 37/62; C07C 227/16; C07C 209/74

(52) U.S. Cl. CPC C07C 37/62; C07C 209/74; C07C 227/16
USPC 562/456; 568/775; 564/412; 564/374; 568/765

(57) ABSTRACT

The invention relates to a process for producing a process for producing an ¹⁸F-labelled compound, the process comprising treating a compound of formula (I)

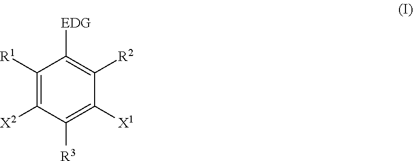

wherein EDG is an electron-donating group selected from —OH, —OR⁴, —NHR⁵ and —NR⁵⁵R⁵; R¹, R², X¹ and X² are as defined herein; and R³ is selected from H, X³ and X⁴, wherein X³ is a monodentate cleavable surrogate group, and X⁴ is a bidentate cleavable surrogate group which is bonded (a) to said X¹ or X² and (b) to the ring carbon atom para to EDG;
with [¹⁸F]fluoride in the presence of an oxidant, thereby producing, when R³ in the compound of formula (I) is H, an ¹⁸F-labelled compound of formula (II), wherein EDG is as defined above and R¹, R², X¹ and X² are as defined herein; or thereby producing, when R³ in the compound of formula (I) is said monodentate cleavable surrogate group X³, a compound of formula (IIa), wherein EDG′ is O, NR⁵, —NR⁵⁵R⁵ or [OR⁴]⁺, and wherein R⁴, R⁵, R⁵⁵, R¹, R², X¹, X² and X³ are as defined herein; or thereby producing, when R³ in the compound of formula (I) is said bidentate cleavable surrogate group X⁴, a compound of formula (IIc) or a compound of formula (IId), wherein EDG′ is O, NR⁵, —NR⁵⁵R⁵ or [OR⁴]⁺, and wherein R⁴, R⁵, R⁵⁵, R¹, R², X¹, X² and X⁴ are as defined herein

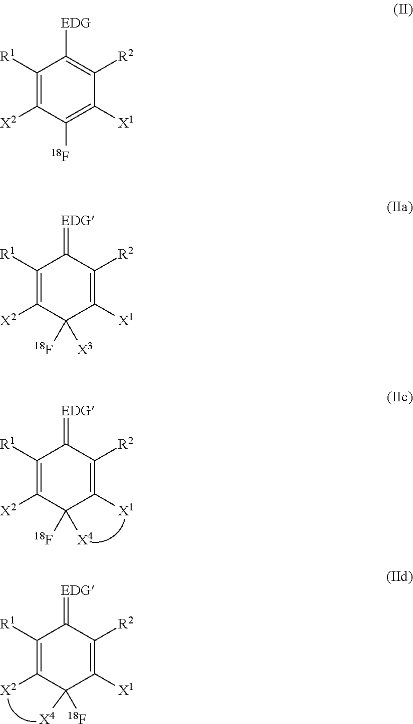

### FIELD OF THE INVENTION

The invention relates to a process for producing compounds labelled with ¹⁸F suitable for use in Positron Emission Tomography (PET).

### BACKGROUND OF THE INVENTION

Positron Emission Tomography (PET) is a nuclear imaging technique of ever increasing importance in diagnostic medicine today. It allows non-invasive diagnostic examination of subjects via the detection of pairs of gamma rays indirectly emitted from positron emitting radioisotopes, producing a 3D image of a functional process in vivo. PET requires the use of a positron emitting radionuclide to trace a physiological or biochemical process in tissue. In order to take a PET scan, a short half-life radionuclide which decays through positron emission is incorporated into a metabolically active molecule. This is injected into the patient and allowed to circulate round the body in order to obtain its optimum biodistribution. The subject is then placed within the PET scanner. A relatively accurate image can be drawn of the radiotracer distribution within the area of interest.

PET most commonly utilizes the radioactive forms of carbon (¹⁰C), nitrogen (¹³N), oxygen (¹⁵O) and fluorine (¹⁸F). Use of these isotopes allows the labelling of many different substrates without altering the biological activity. The half lives of these nuclei are relatively short, which poses a time-scale problem for radio-chemists and can leave little room for manoeuvre between introducing the radioisotope into the tracer, and conducting the PET scan. Of these isotopes ¹⁸F has the most convenient (longest) half life, of 109.7 minutes.

Positron emitting ¹⁸F can be reliably produced on large scale as ¹⁸F⁻. This can then be used to fluorinate in its nucleophilic fluoride form. The majority of nucleophilic fluorinations utilize the no-carrier added ¹⁸F-fluoride ion. Once the nucleophilic source of ¹⁸F⁻ has been produced, fluorination of a compound typically involves the activation of the no-carrier added fluoride by the addition of a cryptand (typically Kryptofix-222) to form a ‘naked fluoride ion’ as a K[¹⁸F]F—K₂₂₂ complex. Alternatively, [¹⁸F]tetrabutylammonium fluoride ([¹⁸F]TBAF) and [¹⁸F]cesium fluoride ([¹⁸F]CsF) can be used as sources of nucleophilic ¹⁸F-fluoride. [¹⁸F]TBAF and [¹⁸F]CsF are typically prepared by trapping ¹⁸F⁻ on an ion exchange column and eluting with tetrabutylammoniumhydrogencarbonate and Cs₂CO₃ respectively.

Alternatively, ¹⁸F⁻ can undergo further manipulation to convert it into one of a number of electrophilic fluorinating reagents. The most common of these electrophilic reagents is [¹⁸F]F₂. After its initial production, electrophilic fluorination with [¹⁸F]F₂ can be performed directly, the most common reactions being electrophilic aromatic substitutions of trialkyl tin or mercury groups. A major drawback of electrophilic radiofluorination however is that only one of the two atoms in elemental fluorine is positron-emitting ¹⁸F, and so use of [¹⁸F]F₂ either to fluorinate a species directly or to produce other fluorination reagents can only lead to a theoretical maximum radiochemical yield of 50%. This, combined with a low specific activity, means that electrophilic radiofluorination is only used when a nucleophilic method is not feasible.

Aromatic fluorine is often found in many drug molecules due to its metabolic stability towards oxidation and degradation, thus improving the drugs' pharmacokinetic profile. However, ¹⁸F-fluorination of electron-rich aromatics is only possible via electrophilic fluorination methods, using low specific activity [¹⁸F]F₂. Thus, unactivated aromatic rings which have not been activated with an electron withdrawing group, and aromatic rings which bear an electron donating group (such as hydroxyl or amino) can currently only be fluorinated directly using electrophilic fluorination. Such methods have the disadvantages mentioned above, including low radiochemical yield.

Although nucleophilic fluorination has been used in the past in, its use has been limited to special precursor types and multi-step procedures. For instance, electron-rich aromatics such as 4-fluorophenol and 4-fluoroaniline are very useful prosthetic groups for ¹⁸F-radiosynthesis. However, current selected methods of synthesis for 4-[¹⁸F]fluorophenol using nucleophilic fluoride involve elaborate precursors (e.g. iodonium salts), harsh conditions (high temperature and/or pressure), multi-step synthesis and/or non-selective synthesis via the Baeyer-Villiger reaction (see FIG. 1(a)). Similarly, 4-[¹⁸F]fluoroaniline is synthesized via a two step procedure by nucleophilic aromatic substitution (S_{N}Ar) of a nitroaryl substrate followed by a hydrogenation (see FIG. 1(b)).

There therefore exists a continuing need to develop a straightforward, one-step method to access unactivated ¹⁸F fluoroaromatics via nucleophilic fluorination. This would inevitably have a significant impact on drug discovery and PET.

### SUMMARY OF THE INVENTION

The invention provides a simple, direct process for producing ¹⁸F-labelled fluoroaromatics via nucleophilic fluorination of electron-rich aromatics. The process enables useful prosthetic groups (such as 4-[¹⁸F]fluorophenol and 4-[¹⁸F]fluoroaniline) to be produced directly from their electron-rich precursors in a “one-pot” synthesis. The inventors have achieved this by performing the nucleophilic radiolabelling reaction in the presence of an oxidant. Without wishing to be bound by theory, it is thought that the oxidant oxidises the electron-rich aromatic ring prior to radiolabelling in order to facilitate nucleophilic attack of [¹⁸F]fluoride.

Accordingly, the invention provides a process for producing an ¹⁸F-labelled compound, the process comprising:

treating a compound of formula (I)

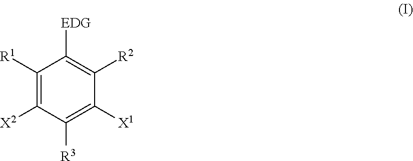

wherein

EDG is an electron-donating group selected from —OH, —OR⁴, —NHR⁵ and —N(R⁵⁵)(R⁵);

R⁴ is unsubstituted or substituted C₁₋₂₀ alkyl, unsubstituted or substituted acyl, unsubstituted or substituted aryl, unsubstituted or substituted C₃₋₁₀ cycloalkyl, or —SiR⁶⁶R⁶R⁷; wherein R⁶⁶, R⁶ and R⁷, which are the same or different, are independently selected from H, unsubstituted or substituted C₁₋₂₀ alkyl, unsubstituted or substituted aryl, and unsubstituted or substituted C₃₋₁₀ cycloalkyl, and unsubstituted or substituted C₁₋₂₀ alkoxy;

R⁵ is selected from —C(O)OR⁸, —S(O)₂R⁹, unsubstituted or substituted C₁₋₂₀ alkyl, unsubstituted or substituted aryl, unsubstituted or substituted C₃₋₁₀ cycloalkyl, acyl, and —SiR⁶⁶R⁶R⁷, provided that R⁵ and R¹ or R⁵ and R² may together form a bidentate group L² wherein L² is -alk-, —C(O)-alk-, —C(O)O-alk- or —S(O)₂-alk- wherein -alk- is unsubstituted or substituted C₁₋₃ alkylene; wherein R⁶⁶, R⁶ and R⁷, which are the same or different, are independently selected from H, unsubstituted or substituted C₁₋₂₀ alkyl, unsubstituted or substituted aryl, unsubstituted or substituted C₃₋₁₀ cycloalkyl, and unsubstituted or substituted C₁₋₂₀ alkoxy; wherein R⁸ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R⁹ is unsubstituted or substituted aryl or unsubstituted or substituted C₁₋₂₀ alkyl;

R⁵⁵ is selected from —C(O)OR⁸, —S(O)₂R⁹, unsubstituted or substituted C₁₋₂₀ alkyl, unsubstituted or substituted aryl, unsubstituted or substituted C₃₋₁₀ cycloalkyl, acyl, and —SiR⁶⁶R⁶R⁷; wherein R⁶⁶, R⁶ and R⁷, which are the same or different, are independently selected from H, unsubstituted or substituted C₁₋₂₀ alkyl, unsubstituted or substituted aryl, unsubstituted or substituted C₃₋₁₀ cycloalkyl, and unsubstituted or substituted C₁₋₂₀ alkoxy; wherein R⁸ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R⁹ is unsubstituted or substituted aryl or unsubstituted or substituted C₁₋₂₀ alkyl;

R¹ and R², which are the same or different, are independently selected from H, unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, acyl, amido, acylamido, halo, cyano, —OR¹⁰ and —NR¹¹R¹¹,

provided that when EDG is —NHR⁵ or —N(R⁵⁵)(R⁵), R⁵ and R¹ or R⁵ and R² may together form a bidentate group L² wherein L² is -alk-, —C(O)-alk-, —C(O)O-alk- or —S(O)₂-alk- wherein -alk- is unsubstituted or substituted C₁₋₃ alkylene,

and provided that R¹ and X² may together form a bidentate group such that R¹, X² and the ring carbon atoms to which R¹ and X² are bonded together form an unsubstituted or substituted fused aryl, heteroaryl, C₅₋₈-carbocyclic or C₅₋₈ heterocyclic ring;

and provided that R² and X¹ may together form a bidentate group such that R², X¹ and the ring carbon atoms to which R² and X¹ are bonded together form an unsubstituted or substituted fused aryl, heteroaryl, C₅₋₈ carbocyclic or C₅₋₈ heterocyclic ring;

R¹⁰ is a hydroxyl protecting group;

R¹¹ and R¹¹¹, which are the same or different, are independently selected from unsubstituted or substituted C₁₋₂₀ alkyl, unsubstituted or substituted C₃₋₁₀ cycloalkyl, C₁₋₂₀ perfluoroalkyl, acyl, unsubstituted or substituted aryl, perfluoroaryl, unsubstituted or substituted heteroaryl, unsubstituted or substituted C₃₋₁₀ heterocyclyl, —C(O)OR¹⁶ and —S(O)₂R¹⁷, wherein R¹⁶ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R¹⁷ is unsubstituted or substituted aryl or unsubstituted or substituted C₁₋₁₀ alkyl;

X¹ and X², which are the same or different, are independently selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, unsubstituted or substituted C₁₋₂₀ alkoxy, amino, unsubstituted or substituted C₁₋₁₀ alkylamino, unsubstituted or substituted di(C₁₋₁₀)alkylamino, unsubstituted or substituted acyl, unsubstituted or substituted amido, unsubstituted or substituted acylamido, halo, cyano and a group of formula (X), formula (X2), formula (Y), formula (Z1) or formula (Z2)

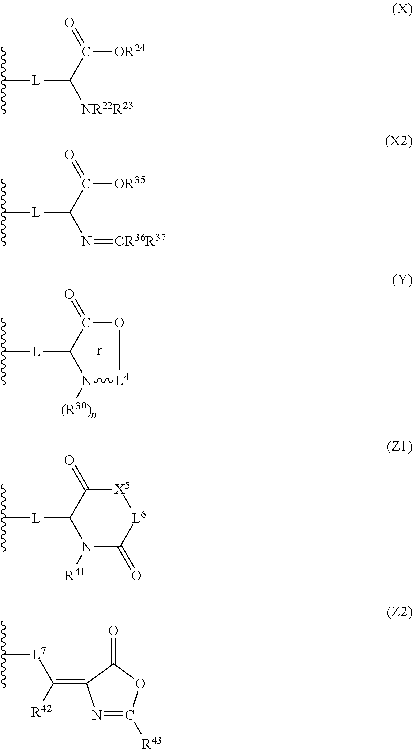

wherein

L⁵ is unsubstituted or substituted C₁₋₆ alkylene;

R⁴⁰ is an amino protecting group;

L is unsubstituted or substituted C₁₋₄ alkylene;

R²² and R²³, which are the same or different, are independently selected from H and an amino protecting group;

R²⁴ is H or a carboxyl protecting group;

R³⁵ is H or a carboxyl protecting group;

R³⁶ and R³⁷, which are the same or different, are independently selected from unsubstituted or substituted aryl, unsubstituted or substituted heteroaryl, unsubstituted or substituted C₃₋₁₀ heterocyclyl, unsubstituted or substituted C₁₋₂₀ alkyl, or unsubstituted or substituted C₃₋₁₀ cycloalkyl, provided that R³⁶ and R³⁷ may together form an unsubstituted or substituted C₄. alkylene alkylene group;

R³⁰ is H, unsubstituted or substituted C₁₋₁₀ alkyl, or unsubstituted or substituted aryl;

n is 0 or 1, provided that when n is 0, the bond between L⁴ and N is a double bond and when n is 1, the bond between L⁴ and N is a single bond;

L⁴ is a linking group wherein L⁴ forms, together with the —N(R³⁰)ₙ—C(L)-C(O)—O— moiety to which L⁴ is bonded, a ring r which is a C₅₋₈ heterocyclic ring or a C₅₋₈ heteroaryl ring;

R⁴¹ is H or an amino protecting group, provided that when R³ is X⁴, R⁴¹ may be a single bond which connects X⁴ to said group of formula (Z1);

X⁵ is NR⁴⁴ or O, wherein R⁴⁴ is selected from unsubstituted or substituted C₁₋₁₀ alkyl, unsubstituted or substituted C₃₋₁₀ cycloalkyl, unsubstituted or substituted aryl, unsubstituted or substituted heteroaryl, and unsubstituted or substituted C₃₋₁₀ heterocyclyl;

L⁶ is substituted or unsubstituted C₁₋₃ alkylene;

L⁷ is a bond or an unsubstituted or substituted C₁₋₄ alkylene group;

R⁴² is H, unsubstituted or substituted C₁₋₁₀ alkyl, or unsubstituted or substituted aryl;

R⁴³ is unsubstituted or substituted aryl, unsubstituted or substituted heteroaryl, unsubstituted or substituted C₃₋₁₀ heterocyclyl, unsubstituted or substituted C₁₋₂₀ alkyl, or unsubstituted or substituted C₃₋₁₀ cycloalkyl;

provided that X² and R¹ may together form a bidentate group such that R¹, X² and the ring carbon atoms to which R¹ and X² are bonded together form an unsubstituted or substituted fused aryl, heteroaryl, C₅₋₈ carbocyclic or C₅₋₈ heterocyclic ring;

and provided that X¹ and R² may together form a bidentate group such that R², X¹ and the ring carbon atoms to which R² and X¹ are bonded together form an unsubstituted or substituted fused aryl, heteroaryl, C₅₋₈ carbocyclic or C₅₋₈ heterocyclic ring;

and provided that when X¹ or X² is substituted C₁₋₂₀ alkyl, substituted -L⁵-N(R⁴⁰)H, substituted C₃₋₂₀ cycloalkyl, substituted aryl, substituted heteroaryl, substituted C₃₋₁₀ heterocyclyl, substituted C₁₋₂₀ alkoxy, substituted C₁₋₁₀ alkylamino, substituted di(C₁₋₁₀)alkylamino, substituted acyl, substituted amido, substituted acylamido, or a group of formula (X), formula (X2), formula (Y), formula (Z1) or formula (Z2), said X¹ or X² may be substituted with a group X⁴, wherein X⁴ is a bidentate cleavable surrogate group which is bonded (a) to said X¹ or X² and (b) to the ring carbon atom para to EDG;

R³ is selected from H, X³ and X⁴, wherein X³ is a monodentate cleavable surrogate group and X⁴ is said bidentate cleavable surrogate group; with [¹⁸F]fluoride in the presence of an oxidant, thereby producing, when R³ in the compound of formula (I) is H, an ¹⁸F-labelled compound of formula (II):

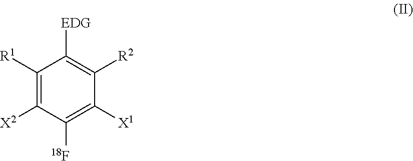

wherein EDG, R¹, R², X¹ and X² are as defined above,

or thereby producing, when R³ in the compound of formula (I) is said monodentate cleavable surrogate group X³, a compound of formula (IIa):

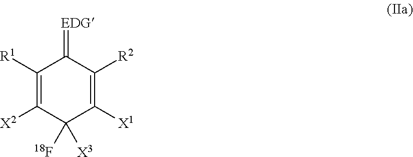

wherein EDG′ is O, NR⁵, [OR⁴]⁺ or [NR⁵⁵R⁵]⁺ and wherein R⁴, R⁵, R⁵⁵, R¹, R², X¹, X² and X³ are as defined above,

or thereby producing, when R³ in the compound of formula (I) is said bidentate cleavable surrogate group X⁴, a compound of formula (IIc) or a compound of formula (IId):

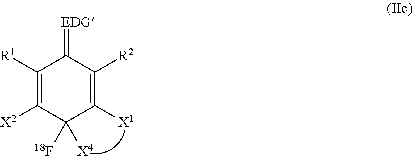

wherein EDG′ is O, NR⁵, [OR⁴]⁺ or [NR⁵⁵R⁵]⁺ and wherein R⁴, R⁵, R⁵⁵, R¹, R² and X² are as defined above; and wherein X¹ is a C₁₋₂₀ alkyl, -L⁵-N(R⁴⁰)H, C₃₋₂₀ cycloalkyl, aryl, heteroaryl, C₃₋₁₀ heterocyclyl, C₁₋₂₀ alkoxy, C₁₋₁₀ alkylamino, di(C₁₋₁₀)alkylamino, acyl, amido or acylamido group, or a group of formula (X), formula (X2), formula (Y), formula (Z1) or formula (Z2), provided that X^{L} is substituted with X⁴, wherein X⁴ is said bidentate cleavable surrogate group which is bonded (a) to X¹ and (b) to the ring carbon atom para to EDG′;

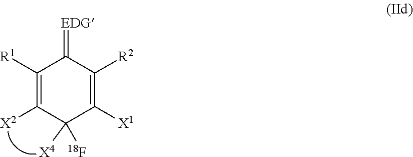

wherein EDG′ is O, NR⁵, [OR⁴]⁺ or [NR⁵⁵R⁵]⁺ and wherein R⁴, R⁵, R⁵⁵, R¹, R² and X¹ are as defined above; and wherein X² is a C₁₋₂₀ alkyl, -L⁵-N(R⁴⁰)H, C₃₋₂₀ cycloalkyl, aryl, heteroaryl, C₃₋₁₀ heterocyclyl, C₁₋₂₀ alkoxy, C₁₋₁₀ alkylamino, di(C₁₋₁₀)alkylamino, acyl, amido or acylamido group, or a group of formula (X), formula (X2), formula (Y), formula (Z1) or formula (Z2), provided that X² is substituted with X⁴, wherein X⁴ is said bidentate cleavable surrogate group which is bonded (a) to X² and (b) to the ring carbon atom para to EDG′.

In one embodiment, the process for producing an ¹⁸F-labelled compound comprises:

treating a compound of formula (I)

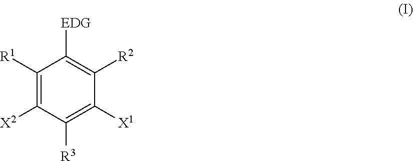

wherein

EDG is an electron-donating group selected from —OH, —OR⁴, —NHR⁵ and —N(R⁵⁵)(R⁵);

R⁴ is unsubstituted or substituted C₁₋₂₀ alkyl, unsubstituted or substituted aryl, unsubstituted or substituted C₃₋₁₀ cycloalkyl, or —SiR⁶⁶R⁶R⁷; wherein R⁶⁶, R⁶ and R⁷, which are the same or different, are independently selected from H, unsubstituted or substituted C₁₋₂₀ alkyl, unsubstituted or substituted aryl, and unsubstituted or substituted C₃₋₁₀ cycloalkyl, and unsubstituted or substituted C₁₋₂₀ alkoxy;

R⁵ is selected from —C(O)OR⁸, —S(O)₂R⁹, unsubstituted or substituted C₁₋₂₀ alkyl, unsubstituted or substituted aryl, unsubstituted or substituted C₃₋₁₀ cycloalkyl, acyl, and —SiR⁶⁶R⁶R⁷, provided that R⁵ and R¹ or R⁵ and R² may together form a bidentate group L² wherein L² is -alk-, —C(O)-alk-, —C(O)O-alk- or —S(O)₂-alk- wherein -alk- is unsubstituted or substituted C₁₋₃ alkylene; wherein R⁶⁶, R⁶ and R⁷, which are the same or different, are independently selected from H, unsubstituted or substituted C₁₋₂₀ alkyl, unsubstituted or substituted aryl, unsubstituted or substituted C₃₋₁₀ cycloalkyl, and unsubstituted or substituted C₁₋₂₀ alkoxy; wherein R⁸ is selected from unsubstituted or substituted C₁₋₁₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R⁹ is unsubstituted or substituted aryl or unsubstituted or substituted C₁₋₂₀ alkyl;

R⁵⁵ is selected from —C(O)OR⁸, —S(O)₂R⁹, unsubstituted or substituted C₁₋₂₀ alkyl, unsubstituted or substituted aryl, unsubstituted or substituted C₃₋₁₀ cycloalkyl, acyl, and —SiR⁶⁶R⁶R⁷; wherein R⁶⁶, R⁶ and R⁷, which are the same or different, are independently selected from H, unsubstituted or substituted C₁₋₂₀ alkyl, unsubstituted or substituted aryl, unsubstituted or substituted C₃₋₁₀ cycloalkyl, and unsubstituted or substituted C₁₋₂₀ alkoxy; wherein R⁸ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R⁹ is unsubstituted or substituted aryl or unsubstituted or substituted C₁₋₂₀ alkyl;

R¹ and R², which are the same or different, are independently selected from H, unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, acyl, amido, acylamido, halo, cyano, —OR¹⁰ and —NR¹¹R¹¹¹,

provided that when EDG is —NHR⁵ or —N(R⁵⁵)(R⁵), R⁵ and R¹ or R⁵ and R² may together form a bidentate group L² wherein L² is -alk-, —C(O)-alk-, —C(O)O-alk- or —S(O)₂-alk- wherein -alk- is unsubstituted or substituted C₁₋₃ alkylene,

and provided that R¹ and X² may together form a bidentate group such that R¹, X² and the ring carbon atoms to which R¹ and X² are bonded together form an unsubstituted or substituted fused aryl, heteroaryl, C₅₋₈ carbocyclic or C₅₋₈ heterocyclic ring;

and provided that R² and X¹ may together form a bidentate group such that R², X¹ and the ring carbon atoms to which R² and X¹ are bonded together form an unsubstituted or substituted fused aryl, heteroaryl, C₅₋₈ carbocyclic or C₅₋₈ heterocyclic ring;

R¹⁰ is a hydroxyl protecting group;

R¹¹ and R¹¹¹, which are the same or different, are independently selected from unsubstituted or substituted C₁₋₂₀ alkyl, unsubstituted or substituted C_{3 -10} cycloalkyl, C₁₋₂₀ perfluoroalkyl, acyl, unsubstituted or substituted aryl, perfluoroaryl, unsubstituted or substituted heteroaryl, unsubstituted or substituted C₃₋₁₀ heterocyclyl, —C(O)OR¹⁶ and —S(O)₂R¹⁷, wherein R¹⁶ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R¹⁷ is unsubstituted or substituted aryl or unsubstituted or substituted C₁₋₁₀ alkyl;

X¹ and X², which are the same or different, are independently selected from H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, unsubstituted or substituted C₁₋₂₀ alkoxy, amino, unsubstituted or substituted C₁₋₁₀ alkylamino, unsubstituted or substituted di(C₁₋₁₀)alkylamino, unsubstituted or substituted acyl, unsubstituted or substituted amido, unsubstituted or substituted acylamido, halo, cyano and a group of formula (X) or formula (Y)

wherein

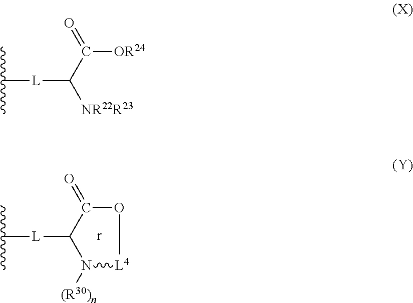

L is unsubstituted or substituted C₁₋₄ alkylene;

R²² and R²³, which are the same or different, are independently selected from H and an amino protecting group;

R²⁴ is H or a carboxyl protecting group;

R³⁰ is H, unsubstituted or substituted C₁₋₁₀ alkyl, or unsubstituted or substituted aryl;

n is 0 or 1, provided that when n is 0, the bond between L⁴ and N is a double bond and when n is 1, the bond between L⁴ and N is a single bond;

L⁴ is a linking group wherein L⁴ forms, together with the —N(R³⁰)ₙ—C(L)-C(O)—O— moiety to which L⁴ is bonded, a ring r which is a C₅₋₈ heterocyclic ring or a C₅₋₈ heteroaryl ring;

provided that X² and R¹ may together form a bidentate group such that R¹, X² and the ring carbon atoms to which R¹ and X² are bonded together form an unsubstituted or substituted fused aryl, heteroaryl, C₅₋₈ carbocyclic or C₅₋₈ heterocyclic ring;

and provided that X¹ and R² may together form a bidentate group such that R², X¹ and the ring carbon atoms to which R² and X¹ are bonded together form an unsubstituted or substituted fused aryl, heteroaryl, C₅₋₈ carbocyclic or C₅₋₈ heterocyclic ring; and provided that when X¹ or X² is substituted C₁₋₂₀ alkyl, substituted C₃₋₂₀ cycloalkyl, substituted aryl, substituted heteroaryl, substituted C₃₋₁₀ heterocyclyl, substituted C₁₋₂₀ alkoxy, substituted C₁₋₁₀ alkylamino, substituted di(C₁₋₁₀)alkylamino, substituted acyl, substituted amido, substituted acylamido, or a group of formula (X) or formula (Y), said X¹ or X² may be substituted with a group X⁴, wherein X⁴ is a bidentate cleavable surrogate group which is bonded (a) to said X¹ or X² and (b) to the ring carbon atom para to EDG;

R³ is selected from H, X³ and X⁴, wherein X³ is a monodentate cleavable surrogate group and X⁴ is said bidentate cleavable surrogate group;

with [¹⁸F]fluoride in the presence of an oxidant, thereby producing, when R³ in the compound of formula (I) is H, an ¹⁸F-labelled compound of formula (II):

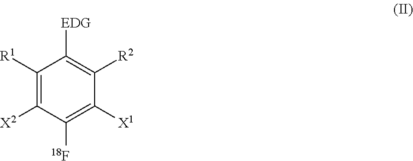

wherein EDG, R¹, R², X¹ and X² are as defined above, or thereby producing, when R³ in the compound of formula (I) is said monodentate cleavable surrogate group X³, a compound of formula (IIa):

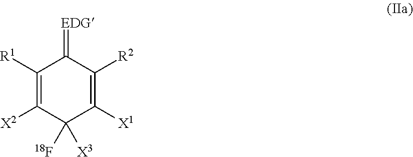

wherein EDG′ is O, NR⁵, [OR⁴]⁺ or [NR⁵⁵R⁵]⁺ and wherein R⁴, R⁵, R⁵⁵, R¹, R², X¹, X² and X³ are as defined above,

or thereby producing, when R³ in the compound of formula (I) is said bidentate cleavable surrogate group X⁴, a compound of formula (IIc) or a compound of formula (IId):

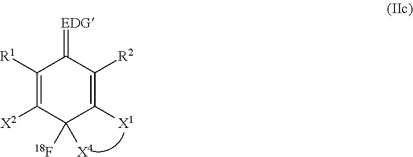

wherein EDG′ is O, NR⁵, [OR⁴]⁺ or [NR⁵⁵R⁵]⁺ and wherein R⁴, R⁵, R⁵⁵, R¹, R² and X² are as defined above; and wherein X¹ is a C₁₋₂₀ alkyl, C₃₋₂₀ cycloalkyl, aryl, heteroaryl, C₃₋₁₀ heterocyclyl, C₁₋₂₀ alkoxy, C₁₋₁₀ alkylamino, di(C₁₋₁₀)alkylamino, acyl, amido or acylamido group, or a group of formula (X) or formula (Y), provided that X¹ is substituted with X⁴, wherein X⁴ is said bidentate cleavable surrogate group which is bonded (a) to X¹ and (b) to the ring carbon atom para to EDG′;

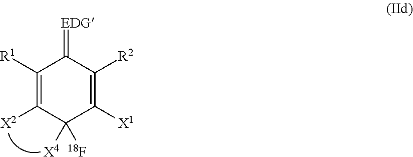

wherein EDG′ is O, NR⁵, [OR⁴]⁺ or [NR⁵⁵R⁵]⁺ and wherein R⁴, R⁵, R⁵⁵, R¹, R² and X¹ are as defined above; and wherein X² is a C₁₋₂₀ alkyl, C₃₋₂₀ cycloalkyl, aryl, heteroaryl, C₃₋₁₀ heterocyclyl, C₁₋₂₀ alkoxy, C₁₋₁₀ alkylamino, di(C₁₋₁₀)alkylamino, acyl, amido or acylamido group, or a group of formula (X) or formula (Y), provided that X² is substituted with X⁴, wherein X⁴ is said bidentate cleavable surrogate group which is bonded (a) to X² and (b) to the ring carbon atom para to EDG′.

Typically, when R³ in the compound of formula (I) is said monodentate cleavable surrogate group X³, the process further comprises rearomatization of the compound of formula (IIa) to produce a compound of formula (II)

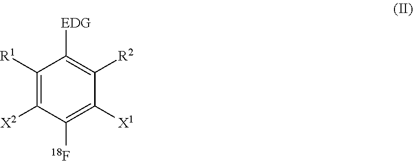

wherein EDG, R¹, R², X¹ and X² are as defined above. The rearomatization is typically performed in situ, in the presence of a reagent which effects cleavage of X³ from the compound of formula (IIa) to produce a compound of formula (II).

Similarly, when R³ in the compound of formula (I) is said bidentate cleavable surrogate group X⁴, the process typically further comprises (i) rearomatization of said compound of formula (IIc) or (IId), comprising cleavage of X⁴ from the ring carbon atom para to EDG′ in said compound. In this embodiment, the process may also comprise (ii) cleavage of X⁴ from the group X¹ or X² to which X⁴ is bonded, thereby producing a compound of the following formula (II):

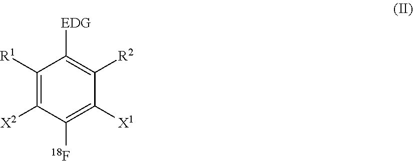

wherein EDG, R¹ and R² are as defined above; and

one of X¹ and X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, unsubstituted or substituted C₁₋₂₀ alkoxy, amino, unsubstituted or substituted C₁₋₁₀ alkylamino, unsubstituted or substituted di(C₁₋₁₀)alkylamino, unsubstituted or substituted acyl, unsubstituted or substituted amido, unsubstituted or substituted acylamido, halo, cyano and a group of formula (X), formula (X2), formula (Y), formula (Z1) or formula (Z2) as defined above; and

the other of X¹ and X² is selected from unsubstituted or substituted C₁₋₂₀ alkyl, unsubstituted or substituted -L⁵-N(R⁴⁰)H, unsubstituted or substituted C₃₋₂₀ cycloalkyl, unsubstituted or substituted aryl, unsubstituted or substituted heteroaryl, unsubstituted or substituted C₃₋₁₀ heterocyclyl, unsubstituted or substituted C₁₋₂₀ alkoxy, unsubstituted or substituted C₁₋₁₀ alkylamino, unsubstituted or substituted di(C₁₋₁₀)alkylamino, unsubstituted or substituted acyl, unsubstituted or substituted amido, unsubstituted or substituted acylamido, and a group of formula (X), formula (X2), formula (Y), formula (Z1) or formula (Z2) as defined above.

When EDG is —NHR⁵ the process may or may not further comprise a deprotection step in which H is substituted for R⁵, thereby producing a compound wherein EDG is —NH₂.

When EDG is —OR⁴ the process may or may not further comprise a deprotection step in which H is substituted for R⁴, thereby producing a compound wherein EDG is —OH.

When R¹ or R² is —OR¹⁰ the process may or may not further comprise a deprotection step in which H is substituted for R¹⁰, thereby producing a compound wherein said R¹ or R² is —OH.

### BRIEF DESCRIPTION OF THE FIGURES

FIG. 1 shows schematically the syntheses of 4-[¹⁸F]fluorophenol ([¹⁸F]3-93) and 4-[¹⁸F]fluoroaniline ([¹⁸F]3-98) by prior art methods.

FIG. 2 shows schematically the one-pot synthesis of ¹⁸F-fluorophenol from 4-tert-butylphenol in accordance with the present invention.

FIG. 3 shows schematically a method of radiolabelling a chiral precursor to 6-¹⁸F-meta-tyrosine. with a microfluidic apparatus (NanoTek®, Advion)

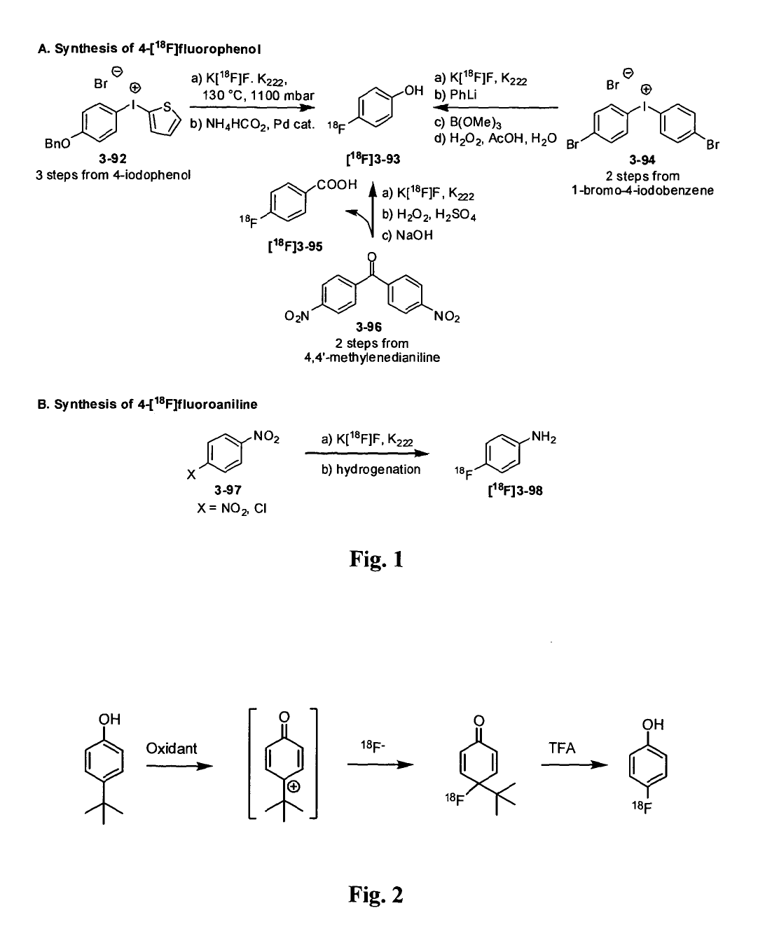

### DETAILED DESCRIPTION OF THE INVENTION

The following substituent definitions apply with respect to the compounds defined herein:

A C₁₋₂₀ alkyl group is an unsubstituted or substituted, straight or branched chain saturated hydrocarbon radical. Typically it is C₁₋₁₀ alkyl, for example methyl, ethyl, propyl, butyl, pentyl, hexyl, heptyl, octyl, nonyl or decyl, or C₁₋₆ alkyl, for example methyl, ethyl, propyl, butyl, pentyl or hexyl, or C₁₋₄ alkyl, for example methyl, ethyl, i-propyl, n-propyl, t-butyl, s-butyl or n-butyl. When an alkyl group is substituted it typically bears one or more substituents selected from substituted or unsubstituted C₁₋₂₀ alkyl, substituted or unsubstituted aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. Examples of substituted alkyl groups include haloalkyl, hydroxyalkyl, aminoalkyl, alkoxyalkyl and alkaryl groups. The term alkaryl, as used herein, pertains to a C₁₋₂₀ alkyl group in which at least one hydrogen atom has been replaced with an aryl group. Examples of such groups include, but are not limited to, benzyl(phenylmethyl, PhCH₂—), benzhydryl (Ph₂CH—), trityl(triphenylmethyl, Ph₃C—), phenethyl(phenylethyl, Ph-CH₂CH₂—), styryl (Ph-CH═CH—), cinnamyl (Ph-CH═CH—CH₂—).

Typically a substituted C₁₋₂₀ alkyl group carries 1, 2 or 3 substituents, for instance 1 or 2.

A C₁₋₂₀ perfluoroalkyl group is a straight or branched chain saturated perfluorinated hydrocarbon radical having from 1 to 20 carbon atoms. Typically, it is a C₁₋₁₀ perfluoroalkyl group, i.e. straight or branched chain saturated perfluorinated hydrocarbon radical having from 1 to 10 carbon atoms. A C₃₋₂₀ perfluoroalkyl group is a straight or branched chain saturated perfluorinated hydrocarbon radical having from 3 to 20 carbon atoms. “Perfluorinated” in this context means completely fluorinated such that there are no carbon-bonded hydrogen atoms replaceable with fluorine. A C₁₋₂₀ or C₃₋₂₀ perfluoroalkyl group may however be substituted with one, two or three perfluoroaryl groups. In such a substituted C₃₋₂₀ perfluoroalkyl group, one, two or three of the carbon-bonded fluorine atoms are replaced with a perfluoroaryl substituent group. Where more than one perfluoroaryl substituent group is present, the perfluoroaryl substituent groups may be bonded to the same or different carbon atoms of the substituted perfluoroalkyl group. Alternatively a C₃₋₂₀ perfluoroalkyl group may be unsubstituted, such that none of the carbon-bonded fluorine atoms is replaced with another group such as a perfluoroaryl group. Typically a C₁₋₂₀ or C₃₋₂₀ perfluoroalkyl group is a is C₃₋₁₂ perfluoroalkyl group. Examples of C₃₋₁₂ perfluoro alkyl groups are perfluoropropyl (C₃) (including perfluoro-n-propyl and perfluoro-iso-propyl), perfluorobutyl (C₄) (including perfluoro-n-butyl, perfluoro-sec-butyl and perfluoro-tert-butyl), perfluoropentyl (C₅), perfluorohexyl (C₆), perfluoroheptyl (C₇), perfluorooctyl (C₈), perfluorononyl (C₉), perfluorodecyl (C₁₀), perfluoroundecyl (C₁₁) and perfluorododecyl (C₁₂), including straight chained and branched isomers thereof. C₁₋₂₀ perfluoroalkyl also of course includes longer-chain perfluoroalkyls, with up to 20 carbon atoms, and shorter ones including —CF₃ and —CF₂—CF₃.

A C₃₋₁₀ cycloalkyl group is an unsubstituted or substituted alkyl group which is also a cyclyl group; that is, a monovalent moiety obtained by removing a hydrogen atom from an alicyclic ring atom of a carbocyclic ring of a carbocyclic compound, which moiety has from 3 to 10 carbon atoms (unless otherwise specified), including from 3 to 10 ring atoms. Thus, the term “cycloalkyl” includes the sub-classes cycloalkyenyl and cycloalkynyl. Examples of groups of C₃₋₁₀o cycloalkyl groups include C₃₋₇ cycloalkyl. When a C₃₋₁₀ cycloalkyl group is substituted it typically bears one or more substituents selected from C₁₋₆ alkyl which is unsubstituted, aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, phosphoric acid, phosphate ester, phosphonic acid and phosphonate ester and sulfonyl. Typically a substituted C₃₋₁₀ cycloalkyl group carries 1, 2 or 3 substituents, for instance 1 or 2.

Examples of C₃₋₁₀ cycloalkyl groups include, but are not limited to, those derived from saturated monocyclic hydrocarbon compounds, which C₃₋₁₀ cycloalkyl groups are unsubstituted or substituted as defined above:

cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀);

unsaturated monocyclic hydrocarbon compounds:

cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C₅);

saturated polycyclic hydrocarbon compounds:

thujane (C₁₀), carane (C₁₀), pinane (C₁₀), bornane (C₁₀), norcarane (C₇), norpinane (C₇), norbornane (C₇), adamantane (C₁₋₁₀), decalin (decahydronaphthalene) (C₁₀);

unsaturated polycyclic hydrocarbon compounds: camphene (C₁₀), limonene (C₁₋₁₀), pinene (C₁₀),

polycyclic hydrocarbon compounds having an aromatic ring:

indene (C₉), indane (e.g., 2,3-dihydro-1H-indene) (C₉), tetraline (1,2,3,4-tetrahydronaphthalene) (C₁₀).

A C₃₋₁₀ heterocyclyl group is an unsubstituted or substituted monovalent moiety obtained by removing a hydrogen atom from a ring atom of a heterocyclic compound, which moiety has from 3 to 10 ring atoms (unless otherwise specified), of which from 1 to are ring heteroatoms. Preferably, each ring has from 3 to 7 ring atoms, of which from 1 to 4 are ring heteroatoms. When a C₃₋₁₀ heterocyclyl group is substituted it typically bears one or more substituents selected from C₁₋₆ alkyl which is unsubstituted, aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, phosphoric acid, phosphate ester, phosphonic acid and phosphonate ester and sulfonyl. Typically a substituted C₃₋₁₀ heterocyclyl group carries 1, 2 or 3 substituents, for instance 1 or 2.

Examples of groups of heterocyclyl groups include C₅₋₁₀ heterocyclyl, C₃₋₇ heterocyclyl, C₅₋₇ heterocyclyl, and C₅₋₆ heterocyclyl.

Examples of (non-aromatic) monocyclic C₃₋₁₀ heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole)

(C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Examples of C₃₋₁₀ heterocyclyl groups which are also aryl groups are described below as heteroaryl groups.

A C₅₋₈ heterocyclic ring is a closed ring of from 5 to 8 covalently linked atoms, which ring is saturated or unsaturated, wherein at least one of the ring atoms is a multivalent ring heteroatom, for example, nitrogen, phosphorus, silicon, oxygen, or sulfur (though more commonly nitrogen, oxygen, or sulfur). Typically, the C₅₋₈ heterocyclic ring is not an aromatic ring. Typically, the C₅₋₈ heterocyclic ring has from 1 to 4 heteroatoms, the remainder of the ring atoms are carbon. Typically, the C₅₋₈ heterocyclic ring is a C₅₋₆ heterocyclic ring in which from 1 to 4 of the ring atoms are ring heteroatoms, and the remainder of the ring atoms are carbon atoms. In this context, the prefixes C₅₋₁₀ and C₅₋₆ denote the number of ring atoms, or range of number of ring atoms. When a C₅₋₁₀ heterocyclic ring is substituted it typically bears one or more substituents selected from those listed above for C₁₋₂₀ alkyl groups.

Examples of monocyclic C₅₋₁₀ heterocyclic rings include, but are not limited to:

N₁: pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

An aryl group is a substituted or unsubstituted, monocyclic or bicyclic aromatic group which typically contains from 6 to 14 carbon atoms, preferably from 6 to 10 carbon atoms in the ring portion. Examples include phenyl, naphthyl, indenyl and indanyl groups. An aryl group is unsubstituted or substituted. When an aryl group as defined above is substituted it typically bears one or more substituents selected from C₁-C₆ alkyl which is unsubstituted (to form an aralkyl group), aryl which is unsubstituted, cyano, amino, C₁₋₁₀-alkylamino, di(C₁₋₁₀)alkylamino, arylamino, diarylamino, arylalkylamino, amido, acylamido, hydroxy, halo, carboxy, ester, acyl, acyloxy, C₁₋₂₀ alkoxy, aryloxy, haloalkyl, sulfhydryl (i.e. thiol, —SH), C₁₋₁₀ alkylthio, arylthio, sulfonic acid, phosphoric acid, phosphate ester, phosphonic acid and phosphonate ester and sulfonyl. Typically it carries 0, 1, 2 or 3 substituents. A substituted aryl group may be substituted in two positions with a single C₁₋₆ alkylene group, or with a bidentate group represented by the formula —X—C₁₋₆ alkylene, or —X—C₁₋₆ alkylene-X—, wherein X is selected from O, S and NR, and wherein R is H, aryl or C₁₋₆ alkyl. Thus a substituted aryl group may be an aryl group fused with a cycloalkyl group or with a heterocyclyl group. The term aralkyl as used herein, pertains to an aryl group in which at least one hydrogen atom (e.g., 1, 2, 3) has been substituted with a C₁₋₆ alkyl group. Examples of such groups include, but are not limited to, tolyl (from toluene), xylyl (from xylene), mesityl (from mesitylene), and cumenyl (or cumyl, from cumene), and duryl (from durene).

The ring atoms of an aryl group may include one or more heteroatoms (as in a heteroaryl group). Such an aryl group (a heteroaryl group) is a substituted or unsubstituted mono- or bicyclic heteroaromatic group which typically contains from 6 to 10 atoms in the ring portion including one or more heteroatoms. It is generally a 5- or 6-membered ring, containing at least one heteroatom selected from O, S, N, P, Se and Si. It may contain, for example, 1, 2 or 3 heteroatoms. Examples of heteroaryl groups include pyridyl, pyrazinyl, pyrimidinyl, pyridazinyl, furanyl, thienyl, pyrazolidinyl, pyrrolyl, oxazolyl, oxadiazolyl, isoxazolyl, thiadiazolyl, thiazolyl, isothiazolyl, imidazolyl, pyrazolyl, indolyl (e.g. 3-indolyl), quinolyl and isoquinolyl. A heteroaryl group may be unsubstituted or substituted, for instance, as specified above for aryl. Typically it carries 0, 1, 2 or 3 substituents.

A C₅₋₈ heteroaryl ring is a heteroaromatic ring of from 5 to 8 covalently linked atoms including one or more heteroatoms. The one or more heteroatoms are typically selected from nitrogen, phosphorus, silicon, oxygen and sulfur (more commonly from nitrogen, oxygen and sulfur). A C₅₋₈ heteroaryl ring is typically a 5- or 6-membered ring (i.e. a C₅₋₆ heteroaryl ring) containing at least one heteroatom selected from nitrogen, phosphorus, silicon, oxygen and sulfur (more commonly selected from nitrogen, oxygen and sulfur). It may contain, for example, 1, 2 or 3 heteroatoms. Examples of heteroaryl rings include pyridine, pyrazine, pyrimidine, pyridazine, furan, thiofuran, pyrazole, pyrrole, oxazole, oxadiazole, isoxazole, thiadiazole, thiazole, isothiazole, imidazole and pyrazole. In this context, the prefixes C₅₋₁₀ and C₅₋₆ denote the number of ring atoms, or range of number of ring atoms.

A perfluoroaryl group is a perfluorinated aromatic group which may be monocyclic or bicyclic and which typically contains from 6 to 14 carbon atoms, preferably from 6 to carbon atoms in the ring portion. “Perfluorinated” in this context means completely fluorinated such that there are no carbon-bonded hydrogen atoms replaceable with fluorine. Examples include perfluorophenyl (—C₆F₅), perfluoronaphthyl (—C₁₀F₇), perfluorobiphenylyl (—C₆F₄—C₆F₅), perfluoroindenyl (—C₉F₆) and perfluoroindanyl (—C₉F₉) groups. A perfluoroaryl group may however be substituted with one, two or three perfluoroalkyl groups, for instance C₁₋₂₀, C₃₋₂₀ and/or C₃₋₁₂ perfluoroalkyl groups. In such a substituted perfluoroaryl group, one, two or three of the carbon-bonded fluorine atoms are replaced with a perfluoroalkyl substituent group. Alternatively a perfluoroaryl group may be unsubstituted, such that none of the carbon-bonded fluorine atoms is replaced with another group such as a perfluoroalkyl group.

A C₁₋₂₀ alkylene group is an unsubstituted or substituted bidentate moiety obtained by removing two hydrogen atoms, either both from the same carbon atom, or one from each of two different carbon atoms, of a hydrocarbon compound having from 1 to 20 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below. Typically it is C₁₋₁₀ alkylene, for instance C₁₋₆ alkylene. Typically it is C₁₋₄ alkylene, for example methylene, ethylene, i-propylene, n-propylene, t-butylene, s-butylene or n-butylene. It may also be pentylene, hexylene, heptylene, octylene and the various branched chain isomers thereof. An alkylene group may be unsubstituted or substituted, for instance, as specified above for alkyl. Typically a substituted alkylene group carries 1, 2 or 3 substituents, for instance 1 or 2.

In this context, the prefixes (e.g., C₁₋₄, C₁₋₇, C₁₋₂₀, C₂₋₇, C₃₋₇, etc.) denote the number of carbon atoms, or range of number of carbon atoms. For example, the term “C₁₋₄ alkylene,” as used herein, pertains to an alkylene group having from 1 to 4 carbon atoms. Examples of groups of alkylene groups include C₁₋₄ alkylene (“lower alkylene”), C₁₋₇ alkylene, C₁₋₁₀ alkylene and C₁₋₂₀ alkylene.

Examples of linear saturated C₁₋₇ alkylene groups include, but are not limited to, —(CH₂)ₙ— where n is an integer from 1 to 7, for example, —CH₂— (methylene), —CH₂CH₂-(ethylene), —CH₂CH₂CH₂— (propylene), and —CH₂CH₂CH₂CH₂— (butylene).

Examples of branched saturated C₁₋₇ alkylene groups include, but are not limited to, —CH(CH₃)—, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₁₋₇ alkylene groups include, but is not limited to, —CH═CH— (vinylene), —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, and —CH═CH—CH₂—CH₂—CH═CH—.

Examples of branched partially unsaturated C₁₋₇ alkylene groups include, but is not limited to, —C(CH₃)═CH—, —C(CH₃)═CH—CH₂—, and —CH═CH—CH(CH₃)—.

Examples of alicyclic saturated C₁₋₇ alkylene groups include, but are not limited to, cyclopentylene (e.g., cyclopent-1,3-ylene), and cyclohexylene (e.g., cyclohex-1,4-ylene).

Examples of alicyclic partially unsaturated C₁₋₇ alkylene groups include, but are not limited to, cyclopentenylene (e.g., 4-cyclopenten-1,3-ylene), cyclohexenylene (e.g., 2-cyclohexen-1,4-ylene; 3-cyclohexen-1,2-ylene; 2,5-cyclohexadien-1,4-ylene).

C₁₋₂₀ alkylene and C₁₋₂₀ alkyl groups as defined herein are either uninterrupted or interrupted by one or more heteroatoms or heterogroups, such as S, O or N(R″) wherein R″ is H, C₁₋₆ alkyl or aryl (typically phenyl), or by one or more arylene (typically phenylene) groups, or by one or more —C(O)— or —C(O)N(R″)— groups. The phrase “optionally interrupted” as used herein thus refers to a C₁₋₂₀ alkyl group or an alkylene group, as defined above, which is uninterrupted or which is interrupted between adjacent carbon atoms by a heteroatom such as oxygen or sulfur, by a heterogroup such as N(R″) wherein R″ is H, aryl or C₁-C₆ alkyl, or by an arylene group, or by a —C(O)— or —C(O)N(R″)— group, again wherein R″ is H, aryl or C₁-C₆ alkyl.

For instance, a C₁₋₂₀ alkyl group such as n-butyl may be interrupted by the heterogroup N(R″) as follows: —CH₂N(R″)CH₂CH₂CH₃, —CH₂CH₂N(R″)CH₂CH₃, or —CH₂CH₂CH₂N(R″)CH₃. Similarly, an alkylene group such as n-butylene may be interrupted by the heterogroup N(R″) as follows: —CH₂N(R″)CH₂CH₂CH₂—, —CH₂CH₂N(R″)CH₂CH₂—, or —CH₂CH₂CH₂N(R″)CH₂—. Typically an interrupted group, for instance an interrupted C₁₋₂₀ alkylene or C₁₋₂₀ alkyl group, is interrupted by 1, 2 or 3 heteroatoms or heterogroups or by 1, 2 or 3 arylene (typically phenylene) groups. More typically, an interrupted group, for instance an interrupted C₁₋₂₀ alkylene or C₁₋₂₀ alkyl group, is interrupted by 1 or 2 heteroatoms or heterogroups or by 1 or 2 arylene (typically phenylene) groups. For instance, a C₁₋₂₀ alkyl group such as n-butyl may be interrupted by 2 heterogroups N(R″) as follows: —CH₂N(R″)CH₂N(R″)CH₂CH₃.

An arylene group is an unsubstituted or substituted bidentate moiety obtained by removing two hydrogen atoms, one from each of two different aromatic ring atoms of an aromatic compound, which moiety has from 5 to 14 ring atoms (unless otherwise specified). Typically, each ring has from 5 to 7 or from 5 to 6 ring atoms. An arylene group may be unsubstituted or substituted, for instance, as specified above for aryl.

In this context, the prefixes (e.g., C₅₋₂₀, C₆₋₂₀, C₅₋₁₄, C₅₋₇, C₅₋₆, etc.) denote the number of ring atoms, or range of number of ring atoms, whether carbon atoms or heteroatoms. For example, the term “C₅₋₆arylene,” as used herein, pertains to an arylene group having 5 or 6 ring atoms. Examples of groups of arylene groups include C₅₋₂₀ arylene, C₆₋₂₀ arylene, C₅₋₁₄ arylene, C₆₋₁₄ arylene, C₆₋₁₀ arylene, C₅₋₁₂ arylene, C₅₋₁₀ arylene, C₅₋₇ arylene, C₅₋₆arylene, C₅ arylene, and C₆ arylene.

The ring atoms may be all carbon atoms, as in “carboarylene groups” (e.g., C₆₋₂₀ carboarylene, C₆₋₁₄ carboarylene or C₆₋₁₀ carboarylene).

Examples of C₆₋₂₀ arylene groups which do not have ring heteroatoms (i.e., C₆₋₂₀ carboarylene groups) include, but are not limited to, those derived from the compounds discussed above in regard to aryl groups, e.g. phenylene, and also include those derived from aryl groups which are bonded together, e.g. phenylene-phenylene (diphenylene) and phenylene-phenylene-phenylene (triphenylene).

Alternatively, the ring atoms may include one or more heteroatoms, as in “heteroarylene groups” (e.g., C₅₋₁₀ heteroarylene).

Examples of C₅₋₁₀ heteroarylene groups include, but are not limited to, those derived from the compounds discussed above in regard to heteroaryl groups.

A perfluoroarylene group is a perfluorinated bidentate arylene moiety, which moiety has from 5 to 14 ring atoms (unless otherwise specified). “Perfluorinated” in this context means completely fluorinated such that there are no carbon-bonded hydrogen atoms replaceable with fluorine. Examples include perfluorophenylene (—C₆F₄—), perfluoronaphthylene (—C₁₀F₆—) and perfluorobiphenylene (—C₆F₄—C₆F₄—) groups. Typically, a perfluoroarylene group, as specified herein is a perfluorophenylene group (—C₆F₄—).

C₁₋₂₀, C₃₋₂₀ and C₃₋₁₂ perfluoroalkyl groups as defined herein are either uninterrupted or interrupted by one or more, typically one, two or three, perfluoroarylene groups (typically perfluorophenylene groups). The phrase “optionally interrupted” as used herein may therefore refer to a perfluoroalkyl group, as defined above, which is uninterrupted or which is interrupted between adjacent carbon atoms by one or more, typically one, two or three, perfluoroarylene groups (typically perfluorophenylene groups). Unless otherwise specified, the perfluoroalkyl group is usually uninterrupted.

For instance, a C₁₋₂₀ perfluoroalkyl group such as n-perfluorobutyl may be interrupted by one perfluoroarylene group, perfluorophenylene (—C₆F₄—), as follows: —CF₂(—C₆F₄—)CF₂CF₂CF₃, —CF₂CF₂(—C₆F₄—)CF₂CF₃, or —CF₂CF₂CF₂(—C₆F₄—)CF₃.

As used herein the term oxo represents a group of formula: ═O

As used herein the term acyl represents a group of formula: —C(═O)R, wherein R is an acyl substituent, for example, a substituted or unsubstituted C₁₋₂₀ alkyl group, a C₁₋₂₀ perfluoroalkyl group, a substituted or unsubstituted C₃₋₁₀ cycloalkyl group, a substituted or unsubstituted C₃₋₁₀ heterocyclyl group, a substituted or unsubstituted aryl group, a perfluoroaryl group, or a a substituted or unsubstituted heteroaryl group. Examples of acyl groups include, but are not limited to, —C(═O)CH₃ (acetyl), —C(═O)CH₂CH₃ (propionyl), —C(═O)C(CH₃)C(t-butyryl), and —C(═O)Ph (benzoyl, phenone).

As used herein the term acyloxy (or reverse ester) represents a group of formula: —OC(═O)R, wherein R is an acyloxy substituent, for example, substituted or unsubstituted C₁₋₂₀ alkyl group, a substituted or unsubstituted C₃₋₂₀ heterocyclyl group, or a substituted or unsubstituted aryl group, typically a C₁₋₆ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

As used herein the term ester (or carboxylate, carboxylic acid ester or oxycarbonyl) represents a group of formula: —C(═O)OR, wherein R is an ester substituent, for example, a substituted or unsubstituted C₁₋₂₀ alkyl group, a substituted or unsubstituted C₃₋₂₀ heterocyclyl group, or a substituted or unsubstituted aryl group (typically a phenyl group). Examples of ester groups include, but are not limited to, —C(═O)OCH₃, —C(═O)OCH₂CH₃, —C(═O)OC(CH₃)₃, and —C(═O)OPh.

As used herein the term amino represents a group of formula —NH₂. The term C₁₋₁₀-alkylamino represents a group of formula —NHR¹ wherein R¹ is a C₁₋₁₀ alkyl group, preferably a C₁₋₆ alkyl group, as defined previously. The term di(C₁₋₁₀)alkylamino represents a group of formula —NR′R″ wherein R′ and R″ are the same or different and represent C₁₋₁₀ alkyl groups, preferably C₁₋₆ alkyl groups, as defined previously. The term arylamino represents a group of formula —NHR′ wherein R″ is an aryl group, preferably a phenyl group, as defined previously. The term diarylamino represents a group of formula —NR′R″ wherein R′ and R″ are the same or different and represent aryl groups, preferably phenyl groups, as defined previously. The term arylalkylamino represents a group of formula —NR′R″ wherein R′ is a C₁₋₁₀ alkyl group, preferably a C₁₋₆ alkyl group, and R″ is an aryl group, preferably a phenyl group.

A halo group is chlorine, fluorine, bromine or iodine (a chloro group, a fluoro group, a bromo group or an iodo group). It is typically chlorine, fluorine or bromine.

As used herein the term amido represents a group of formula: —C(═O)NR′R″, wherein R′ and R″ are independently amino substituents, as defined for di(C₁₋₁₀)alkylamino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R′ and R″, together with the nitrogen atom to which they are attached, form a heterocyclic structure as in, for example, piperidinocarbonyl, morpholinocarbonyl, thiomorpholinocarbonyl, and piperazinocarbonyl.

As used herein the term acylamido represents a group of formula: —NR¹C(═O)R², wherein R¹ is an amide substituent, for example, hydrogen, a C₁₋₂₀ alkyl group, a C₃₋₂₀ heterocyclyl group, an aryl group, preferably hydrogen or a C₁₋₂₀ alkyl group, and R² is an acyl substituent, for example, a C₁₋₂₀ alkyl group, a C₃₋₂₀ heterocyclyl group, or an aryl group, preferably hydrogen or a C₁₋₂₀ alkyl group. Examples of acylamide groups include, but are not limited to, —NHC(═O)CH₃, —NHC(═O)CH₂CH₃, —NHC(═O)Ph, —NHC(═O)C₁₅H₃₁ and —NHC(═O)C₉H₁₉. Thus, a substituted C₁₋₂₀ alkyl group may comprise an acylamido substituent defined by the formula —NHC(═O)—C₁₋₂₀ alkyl, such as —NHC(═O)C₅H₃₁ or —NHC(═O)C₉H₁₉. R¹ and R² may together form a cyclic structure, as in, for example, succinimidyl, maleimidyl, and phthalimidyl:

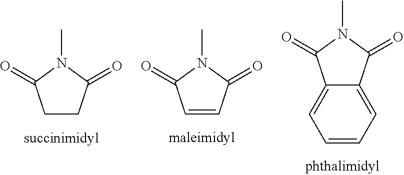

A C₁₋₁₀-alkylthio group is a said C₁₋₁₀ alkyl group, preferably a C₁₋₆ alkyl group, attached to a thio group. An arylthio group is an aryl group, preferably a phenyl group, attached to a thio group.

A C₁₋₂₀ alkoxy group is a said substituted or unsubstituted C₁₋₂₀ alkyl group attached to an oxygen atom. A C₁₋₆ alkoxy group is a said substituted or unsubstituted C₁₋₆ alkyl group attached to an oxygen atom. A C₁₋₄ alkoxy group is a substituted or unsubstituted C₁₋₄ alkyl group attached to an oxygen atom. Said C₁₋₂₀, C₁₋₆ and C₁₋₄ alkyl groups are optionally interrupted as defined herein. Examples of C₁₋₄ alkoxy groups include, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy). Further examples of C₁₋₂₀ alkoxy groups are —O(Adamantyl), —O—CH₂-Adamantyl and —O—CH₂—CH₂-Adamantyl. An aryloxy group is a substituted or unsubstituted aryl group, as defined herein, attached to an oxygen atom. An example of an aryloxy group is —OPh (phenoxy).

Unless otherwise specified, included in the above are the well known ionic, salt, solvate, and protected forms of these substituents. For example, a reference to carboxylic acid or carboxyl group (—COOH) also includes the anionic (carboxylate) form (—COO), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms.

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r- forms; endo- and exo-forms; R—, S—, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto, enol, and enolate forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

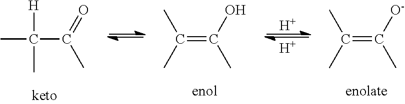

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting known methods, in a known manner.

Unless otherwise specified, a reference to a particular compound also includes ionic, salt, solvated and protected forms.

The process of the invention for producing an ¹⁸F-labelled compound comprises: treating a compound of formula (I)

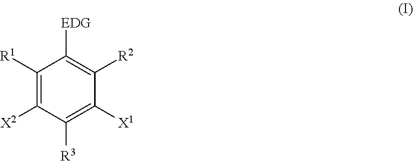

wherein

EDG is an electron-donating group selected from —OH, —OR⁴, —NHR⁵ and —N(R⁵)(R⁵);

R⁴ is unsubstituted or substituted C₁₋₂₀ alkyl, unsubstituted or substituted acyl, unsubstituted or substituted aryl, unsubstituted or substituted C₃₋₁₀ cycloalkyl, or —SiR⁶⁶R⁶R⁷; wherein R⁶⁶, R⁶ and R⁷, which are the same or different, are independently selected from H, unsubstituted or substituted C₁₋₂₀ alkyl, unsubstituted or substituted aryl, and unsubstituted or substituted C₃₋₁₀ cycloalkyl, and unsubstituted or substituted C₁₋₂₀ alkoxy;

R⁵ is selected from —C(O)OR⁸, —S(O)₂R⁹, unsubstituted or substituted C₁₋₂₀ alkyl, unsubstituted or substituted aryl, unsubstituted or substituted C₃₋₁₀ cycloalkyl, acyl, and —SiR⁶⁶R⁶R⁷, provided that R⁵ and R^{L} or R⁵ and R² may together form a bidentate group L², wherein L² is -alk-, —C(O)-alk-, —C(O)O-alk- or —S(O)₂-alk- wherein -alk- is unsubstituted or substituted C₁₋₃ alkylene; wherein R⁶⁶, R⁶ and R⁷, which are the same or different, are independently selected from H, unsubstituted or substituted C₁₋₂₀ alkyl, unsubstituted or substituted aryl, unsubstituted or substituted C₃₋₁₀ cycloalkyl, and unsubstituted or substituted C₁₋₂₀ alkoxy; wherein R⁸ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R⁹ is unsubstituted or substituted aryl or unsubstituted or substituted C₁₋₂₀ alkyl;

R⁵⁵ is selected from —C(O)OR⁸, —S(O)₂R⁹, unsubstituted or substituted C₁₋₂₀ alkyl, unsubstituted or substituted aryl, unsubstituted or substituted C₃₋₁₀ cycloalkyl, acyl, and —SiR⁶⁶R⁶R⁷; wherein R⁶⁶, R⁶ and R⁷, which are the same or different, are independently selected from H, unsubstituted or substituted C₁₋₂₀ alkyl, unsubstituted or substituted aryl, unsubstituted or substituted C₃₋₁₀ cycloalkyl, and unsubstituted or substituted C₁₋₂₀ alkoxy; wherein R⁸ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R⁹ is unsubstituted or substituted aryl or unsubstituted or substituted C₁₋₂₀ alkyl;

R¹ and R², which are the same or different, are independently selected from H, unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, acyl, amido, acylamido, halo, cyano, —OR¹⁰ and —NR¹¹R¹¹,

provided that when EDG is —NHR⁵ or —N(R⁵⁵)(R⁵), R⁵ and R¹ or R⁵ and R² may together form a bidentate group L² wherein L² is -alk-, —C(O)-alk-, —C(O)O-alk- or —S(O)₂-alk- wherein -alk- is unsubstituted or substituted C₁₋₃ alkylene,

and provided that R¹ and X² may together form a bidentate group such that R¹, X² and the ring carbon atoms to which R¹ and X² are bonded together form an unsubstituted or substituted fused aryl, heteroaryl, C₅₋₈ carbocyclic or C₅₋₈ heterocyclic ring;

and provided that R² and X¹ may together form a bidentate group such that R², X¹ and the ring carbon atoms to which R² and X¹ are bonded together form an unsubstituted or substituted fused aryl, heteroaryl, C₅₋₈ carbocyclic or C₅₋₈ heterocyclic ring;

R¹⁰ is a hydroxyl protecting group;

R¹¹ and R¹¹¹, which are the same or different, are independently selected from unsubstituted or substituted C₁₋₂₀ alkyl, unsubstituted or substituted C₃₋₁₀ cycloalkyl, C₁₋₂₀ perfluoroalkyl, acyl, unsubstituted or substituted aryl, perfluoroaryl, unsubstituted or substituted heteroaryl, unsubstituted or substituted C₃₋₁₀ heterocyclyl, —C(O)OR¹⁶ and —S(O)₂R¹⁷, wherein R¹⁶ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R¹⁷ is unsubstituted or substituted aryl or unsubstituted or substituted C₁₋₁₀ alkyl;

X¹ and X², which are the same or different, are independently selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, unsubstituted or substituted C₁₋₂₀ alkoxy, amino, unsubstituted or substituted C₁₋₁₀ alkylamino, unsubstituted or substituted di(C₁₋₁₀)alkylamino, unsubstituted or substituted acyl, unsubstituted or substituted amido, unsubstituted or substituted acylamido, halo, cyano and a group of formula (X), formula (X2), formula (Y), formula (Z1) or formula (Z2)

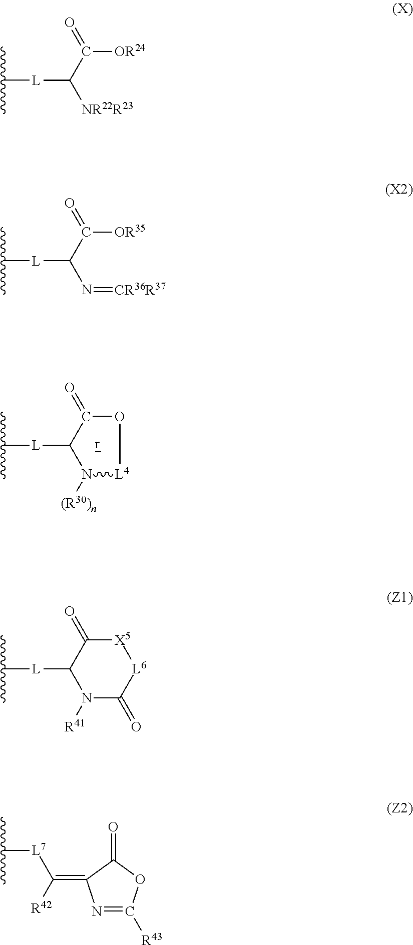

wherein

L⁵ is unsubstituted or substituted C₁₋₆ alkylene;

R⁴⁰ is an amino protecting group;

L is unsubstituted or substituted C₁₋₄ alkylene;

R²² and R²³, which are the same or different, are independently selected from H and an amino protecting group;

R²⁴ is H or a carboxyl protecting group;

R³⁵ is H or a carboxyl protecting group;

R³⁶ and R³⁷, which are the same or different, are independently selected from unsubstituted or substituted aryl, unsubstituted or substituted heteroaryl, unsubstituted or substituted C₃₋₁₀ heterocyclyl, unsubstituted or substituted C₁₋₂₀ alkyl, or unsubstituted or substituted C₃₋₁₀ cycloalkyl, provided that R³⁶ and R³⁷ may together form an unsubstituted or substituted C₄₋₆ alkylene group;

R³⁰ is H, unsubstituted or substituted C₁₋₁₀ alkyl, or unsubstituted or substituted aryl;

n is 0 or 1, provided that when n is 0, the bond between L⁴ and N is a double bond and when n is 1, the bond between L⁴ and N is a single bond;

L⁴ is a linking group wherein L⁴ forms, together with the —N(R³⁰)ₙ—C(L)-C(O)—O— moiety to which L⁴ is bonded, a ring r which is a C₅₋₈ heterocyclic ring or a C₅₋₈ heteroaryl ring;

R⁴¹ is H or an amino protecting group, provided that when R³ is X⁴, R⁴¹ may be a single bond which connects X⁴ to said group of formula (Z1);

X⁵ is NR⁴ or O, wherein R¹⁴ is selected from unsubstituted or substituted C₁₋₁₀ alkyl, unsubstituted or substituted C₃₋₁₀ cycloalkyl, unsubstituted or substituted aryl, unsubstituted or substituted heteroaryl, and unsubstituted or substituted C₃₋₁₀ heterocyclyl;

L⁶ is substituted or unsubstituted C₁₋₃ alkylene;

L⁷ is a bond or an unsubstituted or substituted C₁₋₄ alkylene group;

R⁴² is H, unsubstituted or substituted C₁₋₁₀ alkyl, or unsubstituted or substituted aryl;

R⁴³ is unsubstituted or substituted aryl, unsubstituted or substituted heteroaryl, unsubstituted or substituted C₃₋₁₀ heterocyclyl, unsubstituted or substituted C₁₋₂₀ alkyl, or unsubstituted or substituted C₃₋₁₀ cycloalkyl;

provided that X² and R¹ may together form a bidentate group such that R¹, X² and the ring carbon atoms to which R¹ and X² are bonded together form an unsubstituted or substituted fused aryl, heteroaryl, C₅₋₈ carbocyclic or C₅₋₈ heterocyclic ring;

and provided that X¹ and R² may together form a bidentate group such that R², X¹ and the ring carbon atoms to which R² and X¹ are bonded together form an unsubstituted or substituted fused aryl, heteroaryl, C₅₋₈ carbocyclic or C₅₋₈ heterocyclic ring;

and provided that when X¹ or X² is substituted C₁₋₂₀ alkyl, substituted -L⁵-N(R⁴⁰)H, substituted C₃₋₂₀ cycloalkyl, substituted aryl, substituted heteroaryl, substituted C₃₋₁₀ heterocyclyl, substituted C₁₋₂₀ alkoxy, substituted C₁₋₁₀ alkylamino, substituted di(C₁₋₁₀)alkylamino, substituted acyl, substituted amido, substituted acylamido, or a group of formula (X), formula (X2), formula (Y), formula (Z1) or formula (Z2), said X¹ or X² may be substituted with a group X⁴, wherein X⁴ is a bidentate cleavable surrogate group which is bonded (a) to said X¹ or X² and (b) to the ring carbon atom para to EDG;

R³ is selected from H, X³ and X⁴, wherein X³ is a monodentate cleavable surrogate group and X⁴ is said bidentate cleavable surrogate group;

with [¹⁸F]fluoride in the presence of an oxidant,
thereby producing, when R³ in the compound of formula (I) is H, an ¹⁸F-labelled compound of formula (II):

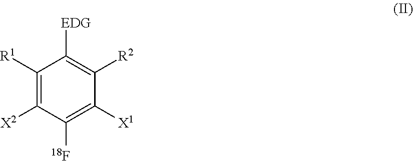

wherein EDG, R¹, R², X¹ and X² are as defined above,

or thereby producing, when R³ in the compound of formula (I) is said monodentate cleavable surrogate group X³, a compound of formula (IIa):

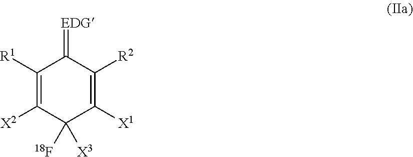

wherein EDG′ is O, NR⁵, [OR⁴]⁺ or [NR⁵⁵R⁵]⁺ and wherein R⁴, R⁵, R⁵⁵, R¹, R², X¹, X² and X³ are as defined above,

or thereby producing, when R³ in the compound of formula (I) is said bidentate cleavable surrogate group X⁴, a compound of formula (IIc) or a compound of formula (IId):

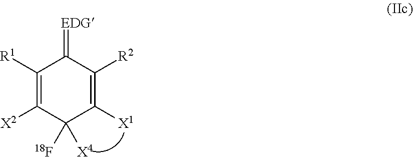

wherein EDG′ is O, NR⁵, [OR⁴]⁺ or [NR⁵⁵R⁵]⁺ and wherein R⁴, R⁵, R⁵⁵, R¹, R² and X² are as defined above; and wherein X¹ is a C₁₋₂₀ alkyl, -L⁵-N(R⁴⁰)H, C₃₋₂₀ cycloalkyl, aryl, heteroaryl, C₃₋₁₀ heterocyclyl, C₁₋₂₀ alkoxy, C₁₋₁₀ alkylamino, di(C₁₋₁₀)alkylamino, acyl, amido or acylamido group, or a group of formula (X), formula (X2), formula (Y), formula (Z1) or formula (Z2), provided that X¹ is substituted with X⁴, wherein X⁴ is said bidentate cleavable surrogate group which is bonded (a) to X¹ and (b) to the ring carbon atom para to EDG′;

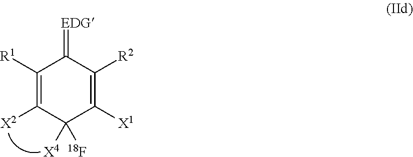

wherein EDG′ is O, NR⁵, [OR⁴]⁺ or [NR⁵⁵R⁵]⁺ and wherein R⁴, R⁵, R⁵⁵, R¹, R² and X¹ are as defined above; and wherein X² is a C₁₋₂₀ alkyl, -L⁵-N(R⁴⁰)H, C₃₋₂₀ cycloalkyl, aryl, heteroaryl, C₃₋₁₀ heterocyclyl, C₁₋₂₀ alkoxy, C₁₋₁₀ alkylamino, di(C₁₋₁₀)alkylamino, acyl, amido or acylamido group, or a group of formula (X), formula (X2), formula (Y), formula (Z1) or formula (Z2), provided that X² is substituted with X⁴, wherein X⁴ is said bidentate cleavable surrogate group which is bonded (a) to X² and (b) to the ring carbon atom para to EDG′.

More typically, the process comprises treating a compound of formula (I)

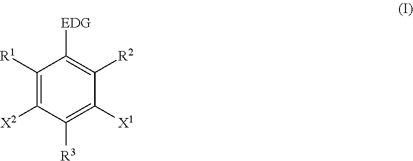

wherein

EDG is an electron-donating group selected from —OH, —OR⁴ and —NHR⁵;

R⁴ is unsubstituted or substituted C₁₋₂₀ alkyl, unsubstituted or substituted aryl, unsubstituted or substituted C₃₋₁₀ cycloalkyl, or —SiR⁶⁶R⁶R⁷; wherein R⁶⁶, R⁶ and R⁷, which are the same or different, are independently selected from H, unsubstituted or substituted C₁₋₂₀ alkyl, unsubstituted or substituted aryl, and unsubstituted or substituted C₃₋₁₀ cycloalkyl, and unsubstituted or substituted C₁₋₂₀ alkoxy;

R⁵ is selected from —C(O)OR⁸, —S(O)₂R⁹, unsubstituted or substituted C₁₋₂₀ alkyl, unsubstituted or substituted aryl, unsubstituted or substituted C₃₋₁₀ cycloalkyl, acyl, and —SiR⁶⁶R⁶R⁷, provided that R⁵ and R¹ or R⁵ and R² may together form a bidentate group L² wherein L² is -alk-, —C(O)-alk-, —C(O)O-alk- or —S(O)₂-alk- wherein -alk- is unsubstituted or substituted C₁₋₃ alkylene; wherein R⁵, R⁶ and R⁷, which are the same or different, are independently selected from H, unsubstituted or substituted C₁₋₂₀ alkyl, unsubstituted or substituted aryl, unsubstituted or substituted C₃₋₁₀ cycloalkyl, and unsubstituted or substituted C₁₋₂₀ alkoxy; wherein R⁸ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R⁹ is unsubstituted or substituted aryl or unsubstituted or substituted C₁₋₂₀ alkyl;

R¹ and R², which are the same or different, are independently selected from H, unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, acyl, amido, acylamido, halo, cyano, —OR¹⁰ and —NR¹¹R¹¹¹, provided that when EDG is NR⁵, R⁵ and R¹ or R⁵ and R² may together form a bidentate group L², wherein L² is -alk-, —C(O)-alk-, —C(O)O-alk- or —S(O)₂-alk- wherein -alk- is unsubstituted or substituted C₁₋₃ alkylene;

R¹⁰ is a hydroxyl protecting group;

R¹¹ and R¹¹¹, which are the same or different, are independently selected from unsubstituted or substituted C₁₋₂₀ alkyl, unsubstituted or substituted C₃₋₁₀ cycloalkyl, C₁₋₂₀ perfluoroalkyl, acyl, unsubstituted or substituted aryl, perfluoroaryl, unsubstituted or substituted heteroaryl, unsubstituted or substituted C₃₋₁₀ heterocyclyl, —C(O)OR¹⁶ and —S(O)₂R¹⁷, wherein R¹⁶ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R¹⁷ is unsubstituted or substituted aryl or unsubstituted or substituted C₁₋₁₀ alkyl;

R³ is selected from H and X³, wherein X³ is a cleavable surrogate group;

X¹ and X², which are the same or different, are independently selected from H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X)

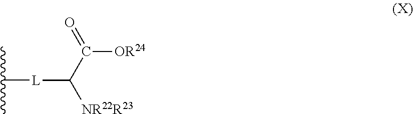

wherein

L is unsubstituted or substituted C₁₋₄ alkylene;

R²² and R²³, which are the same or different, are independently selected from H and an amino protecting group; and

R²⁴ is H or a carboxyl protecting group;

with [¹⁸F]fluoride in the presence of an oxidant,

thereby producing, when R³ in the compound of formula (I) is H, an ¹⁸F-labelled compound of formula (II):

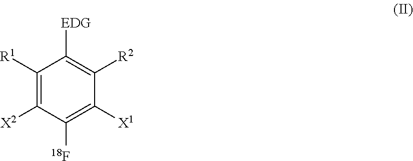

wherein EDG, R¹, R, X¹ and X² are as defined above,

or, when R³ in the compound of formula (I) is said cleavable surrogate group X³, thereby producing a compound of formula (IIa):

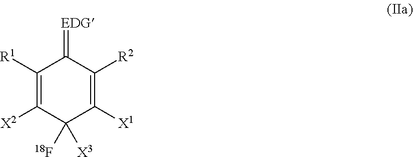

wherein EDG′ is O, NR⁵ or [OR⁴]⁺, and wherein R⁴, R⁵, R¹, R², X¹, X² and X³ are as defined above.

In the processes of the invention for producing an ¹⁸F-labelled compound, the compound of formula (I) is treated with [¹⁸F]fluoride in the presence of an oxidant, thereby fluorinating the compound of formula (I) to produce the ¹⁸F-labelled compound of formula (II), (IIa), (IIc) or (IId). This treatment with [¹⁸F]fluoride may be carried out at room temperature. The treatment with [¹⁸F]fluoride is usually carried out in the presence of a solvent. When a solvent is used, any suitable solvent may be employed. Typically, however, the solvent is a polar aprotic solvent. For instance, the solvent may comprise, or may be, a halogenated organic solvent, acetonitrile, THF or DMSO. The solvent may also comprise a mixture of these solvents, for instance a mixture of any of two of a halogenated organic solvent, acetonitrile, THF and/or DMSO. Typically, the solvent comprises a halogenated organic solvent or acetonitrile. More typically, it comprises an aprotic halogenated organic solvent. Typically, the aprotic halogenated organic solvent is an aprotic chlorinated organic solvent, such as, for instance, dichloromethane, 1,2-dichloroethane, or 1,1,1-trichloroethane. More typically, it is dichloromethane or 1,2-dichloroethane. In one embodiment, the solvent comprises two different aprotic halogenated organic solvents, for instance two different aprotic chlorinated organic solvents. Thus, for instance, the solvent may comprise a mixture of dichloromethane and 1,2-dichloroethane.

Any suitable source of [¹⁸F]fluoride may be used. As will be understood by the skilled person the ¹⁸F⁻ will typically be present in the form of a salt, with a counter cation. Typically, therefore, the process of the invention comprises treating the compound of formula (I) with a salt of ¹⁸F⁻ in the presence of a solvent. Thus, usually the step of treating the compound of formula (I) with [¹⁸F]fluoride comprises treating the compound of formula (I) with a compound comprising ¹⁸F⁻ and a counter cation.

Any suitable counter cation may be used. Typically, the counter cation is a quaternary ammonium cation, for instance tetra-n-butylammonium, or an alkali metal cation, for instance Cs⁺ or K⁺, or a proton, H⁺.

Thus, the step of treating the compound of formula (I) with [¹⁸F]fluoride may comprise treating the compound of formula (I) with: (R³⁰)₄N[¹⁸F]F, wherein each R³⁰, which is the same or different, is independently selected from unsubstituted or substituted C₁₋₂₀ alkyl, unsubstituted or substituted C₃₋₂₀ cycloalkyl, unsubstituted or substituted aryl, unsubstituted or substituted heteroaryl, and unsubstituted or substituted C₃₋₁₀ heterocyclyl. Usually, however, each R³⁰ is unsubstituted C₁₋₁₀ alkyl, more typically unsubstituted C₁₋₆ alkyl, for instance unsubstituted butyl. Thus in one embodiment, each R³⁰ is n-butyl.

Compounds of formula (R³⁰)₄N[¹⁸F]F can be generated by treating (R³⁰)₄NX with [¹⁸F]fluoride, wherein X is a counter anion. In particular, such compounds can be generated by trapping ¹⁸F⁻ on an ion exchange column and eluting with (R³⁰)₄NX. Thus, in one embodiment, the process further comprises generating said (R³⁰)₄N[¹⁸F]F by treating (R³⁰)₄NX with [¹⁸F]fluoride. X may be any suitable counter anion, but typically, X is HCO₃. Accordingly, compounds of formula (R³⁰)₄N[¹⁸F]F are typically generated by treating (R³⁰)₄NHCO₃ with [¹⁸F]fluoride. In particular, such compounds can be generated by trapping ¹⁸F⁻ on an ion exchange column and eluting with (R³⁰)₄NHCO₃. Thus, in one embodiment, the process further comprises generating said (R³⁰)₄N[¹⁸F]F by treating (R³⁰)₄NHCO₃ with [¹⁸F]fluoride.

In another embodiment, the step of treating the compound of formula (I) with [¹⁸F]fluoride comprises treating the compound of formula (I) with: M[¹⁸F]F, wherein M is an alkali metal. Thus, for instance, M may be Li, Na, K or Cs, but it is typically K or Cs. Usually, M is Cs.

Compounds of formula M[¹⁸F]F can be generated by treating a corresponding alkali metal salt, MₙYⁿ⁻, with [¹⁸F]fluoride, wherein Y is a counter anion. In particular, such compounds can be generated by trapping ¹⁸F⁻ on an ion exchange column and eluting with MₙYⁿ⁻. Any suitable counter anion may be employed, but often it is CO₃²⁻. Accordingly, compounds of formula M[¹⁸F]F are typically generated by treating the corresponding alkali metal carbonate, M₂CO₃, with [¹⁸F]fluoride. In particular, such compounds can be generated by trapping ¹⁸F⁻ on an ion exchange column and eluting with M₂CO₃.

Thus, in one embodiment, the process further comprises generating said M[¹⁸F]F by treating M₂CO₃ with [¹⁸F]fluoride, wherein M is said alkali metal. Thus, for instance, M may be Li, Na, K or Cs, but it is typically K or Cs. Usually, M is Cs.

Alternatively, the source of [¹⁸F]fluoride may be [¹⁸F]HF. Thus, the step of treating the compound of formula (I) with [¹⁸F]fluoride may comprise treating the compound of formula (I) with: H[¹⁸F]F.

In another embodiment, the step of treating the compound of formula (I) with [¹⁸F]fluoride comprises treating the compound of formula (I) with: K[¹⁸F]F—K₂₂₂.

In one embodiment, when an alkali metal cation M is employed, the alkali metal cation M is complexed in a cryptand, for instance aminopolyether 2.2.2 (K₂₂₂), which is commercially available as Kryptofix-222. Advantageously, the addition of such a cryptand enables the fluoride ion 18F⁻ to be solubilized in a polar aprotic solvent, for instance acetonitrile. It also enables the formation of a ‘naked fluoride ion’ as a KF—K₂₂₂ complex. In one embodiment, therefore, the source of [¹⁸F]fluoride is M[L¹⁸F]F—K₂₂₂ complex, wherein M is an alkali metal. Thus, for instance, M may be Li, Na, K or Cs, but it is typically K in this embodiment. Thus, the M[¹⁸F]F—K₂₂₂ complex is usually a K[¹⁸F]F—K₂₂₂ complex.

More typically, the step of treating the compound of formula (I) with [¹⁸F]fluoride comprises treating the compound of formula (I) with: [¹⁸F]TBAF (tetrabutylammonium fluoride), or [¹⁸F]CsF.

Thus, in one embodiment, the step of treating the compound of formula (I) with [¹⁸F]fluoride comprises treating the compound of formula (I) with tetra-n-butylammonium[¹⁸F]fluoride or Cs[¹⁸F]F. The tetra-n-butylammonium[¹⁸F]fluoride is typically generated from a mixture of [¹⁸F]fluoride and tetra-n-butylammonium hydrogencarbonate. The Cs[¹⁸]F is typically generated from a mixture of [¹⁸F]fluoride and Cs₂CO₃.

It is thought that the oxidant oxidises the electron-rich aromatic ring of the compound of formula (I) prior to radiolabelling, to facilitate nucleophilic attack of [¹⁸F]fluoride to produce the ¹⁸F-labelled compound of formula (II), (IIa), (IIc) or (IId).

Any suitable oxidant can be used to achieve this. However, hypervalent iodonium (III) reagents have been found to be particularly efficient. Accordingly, the oxidant is typically a hypervalent iodonium (III) reagent. Any hypervalent iodonium (III) reagent may be used. The hypervalent iodonium (III) reagent may for instance be PhI(acetate)₂ or PhI(trifluoroacetate)₂ (PIFA).

Metal oxide oxidants are also useful for the purpose. Accordingly, in one embodiment, the oxidant is a metal oxide. For instance, the oxidant may be MnO₂ or Ag₂O.

The inventors have found that the presence of an additive is desirable but not essential. For instance, it has been observed that no additive is necessary when PhI(trifluoroacetate)₂ (PIFA) is employed as the oxidant.

Typically, however, the step of treating the compound of formula (I) with [¹⁸F]fluoride is performed in the presence of an additive. The additive is typically an acid, but may be a crown ether. Accordingly, in one embodiment, the additive is an acid or a crown ether. Usually, though, the additive is an acid.

Any suitable acid may be used as the additive. Acids which have a pKa which is less than or equal to the pKa of HF are particularly suitable. Thus, in one embodiment, the acid has a pKa less than or equal to the pKa of HF.

The additive may for instance be a mineral acid, a sulfonic acid or an organic acid. Accordingly, in one embodiment, the additive is a mineral acid selected from H₂SO₄, HCl, HNO₃, HBr, HI and HClO₄; a sulfonic acid selected from camphorsulfonic acid (CSA), MeSO₃H and PhSO₃H; or an organic acid selected from p-nitrobenzoic acid and a halogenated organic acid.

Usually, the acid additive is a strong organic acid, for instance p-nitrobenzoic acid or a halogenated organic acid. More typically, the acid used is a halogenated organic acid. Particularly preferred are halogenated organic acids having the formula R³¹—COOH, wherein R³¹ is a C₁₋₁₀o alkyl group substituted with one or more halo groups, for instance one, two or three halo groups, or wherein R³¹ is a C₁₋₁₀ perfluoroalkyl group.

In one embodiment, the additive is trifluoroacetic acid.

In particularly preferred embodiments, the process is performed in a microfluidic reactor. The process of the invention can give particularly high yields of the ¹⁸F-labelled product when performed in a microfluidic reactor.

Thus, in one embodiment of the process of the present invention, said step of treating said compound of formula (I) with said [¹⁸F]fluoride in the presence of said oxidant is performed in a microfluidic reactor.

When performed in a microfluidic reactor, the step of treating said compound of formula (I) with said [¹⁸F]fluoride in the presence of said oxidant typically comprises:

contacting a first solution comprising said compound of formula (I) and said [¹⁸F]fluoride with a second solution comprising said oxidant, in said microfluidic reactor. The oxidant is as defined herein. The second solution typically further comprises said additive.

Typically, the concentration of the compound of formula (I) in said first solution is from about 0.1 M to about 1.0 M, more typically from 0.25 M to 0.5 M.

The concentration of the oxidant in said second solution may also be from about 0.1 M to about 1.0 M, more typically from 0.25 M to 0.5 M.

Typically, when an additive is present and the additive is trifluoroacetic acid, it is present in a concentration of about 3% (v/v).

The solvent employed in said first and second solutions typically comprises a polar aprotic solvent. It usually comprises an aprotic halogenated organic solvent, or a mixture of two or more aprotic halogenated organic solvents. Typically, the aprotic halogenated organic solvent or solvents employed in said first and second solutions are aprotic chlorinated organic solvents, such as, for instance, dichloromethane, 1,2-dichloroethane, or 1,1,1-trichloroethane. Thus, the solvent employed in said first and second solutions typically comprises dichloromethane and/or 1,2-dichloroethane. In one embodiment, the solvent comprises two different aprotic halogenated organic solvents, for instance two different aprotic chlorinated organic solvents. Thus, for instance, the solvent may comprise a mixture of dichloromethane and 1,2-dichloroethane.

Accordingly, when the step of treating said compound of formula (I) with said [¹⁸F]fluoride in the presence of said oxidant is performed in a microfluidic reactor, said first solution typically comprises said compound of formula (I) and a compound comprising ¹⁸F⁻ and a counter cation. Typically, the counter cation is a quaternary ammonium cation, an alkali metal or H⁺.

Usually, said first solution comprises said compound of formula (I) and:

(i) (R³⁰)₄N[¹⁸F]F, wherein each R³⁰, which is the same or different, is independently selected from unsubstituted or substituted C₁₋₂₀ alkyl, unsubstituted or substituted C₃₋₂₀ cycloalkyl, unsubstituted or substituted aryl, unsubstituted or substituted heteroaryl, and unsubstituted or substituted C₃₋₁₀ heterocyclyl, preferably wherein each R³⁰ is n-butyl; or

(ii) (R³⁰)₄NX and [¹⁸F]fluoride, wherein each R³⁰, which is the same or different, is independently selected from unsubstituted or substituted C₁₋₂₀ alkyl, unsubstituted or substituted C₃₋₂₀ cycloalkyl, unsubstituted or substituted aryl, unsubstituted or substituted heteroaryl, and unsubstituted or substituted C₃₋₁₀ heterocyclyl, preferably wherein each R³⁰ is n-butyl, and wherein X is a counter anion, typically wherein X is HCO₃; or

(iii) M[¹⁸F]F, wherein M is an alkali metal, preferably wherein M is Cs; or

(iv) MₙYⁿ⁻ and [¹⁸F]fluoride, wherein M is an alkali metal, preferably wherein M is Cs, and wherein Y is a counter anion, typically wherein Y is CO₃ (in which case said MₙYⁿ⁻ is M₂CO₃); or

(v) H[¹⁸F]F; or

(vi) K[¹⁸F]F—K₂₂₂.

When said first solution comprises said compound of formula (I) and (R³⁰)₄N[¹⁸F]F, the process typically further comprises generating said (R³⁰)₄N[¹⁸F]F in said first solution by treating (R³⁰)₄NHCO₃ with [¹⁸F]fluoride. In these compounds, each R³⁰, which is the same or different, is independently selected from unsubstituted or substituted C₁₋₂₀ alkyl, unsubstituted or substituted C₃₋₂₀ cycloalkyl, unsubstituted or substituted aryl, unsubstituted or substituted heteroaryl, and unsubstituted or substituted C₃₋₁₀ heterocyclyl. Preferably, each R³⁰ is n-butyl,

When said first solution comprises said compound of formula (I) and M[¹⁸F]F, the process typically further comprises generating said M[¹⁸F]F in said first solution by treating M₂CO₃ with [¹⁸F]fluoride. M is an alkali metal, preferably Cs.

In one embodiment, the first solution comprises said compound of formula (I) and tetrabutylammonium[¹⁸F]fluoride. Typically, in this embodiment, the process further comprises generating the tetrabutylammonium[¹⁸F]fluoride from a mixture of [¹⁸F]fluoride and tetrabutylammoniumhydrogencarbonate.

In another embodiment, the first solution comprises said compound of formula (I) and Cs[¹⁸F]F. Typically, in this embodiment, the process further comprises generating the Cs[¹⁸F]F from a mixture of [¹⁸F]fluoride and Cs₂CO₃.

After the fluorination step, the process of the invention may further comprise recovering the compound of formula (II), (IIa), (IIc) or (IId). These compounds can be recovered from the reaction mixture using standard methods for purification of ¹⁸F-labelled compounds, for instance by solid phase extraction and/or HPLC. Accordingly, in one embodiment the process further comprises purifying said compound of formula (II), (IIa), (IIc) or (IId) by solid phase extraction and/or HPLC.

When R³ in the compound of formula (I) is said cleavable surrogate group X³, the process typically further comprises rearomatisation of the compound of formula (IIa) to produce a compound of formula (II)

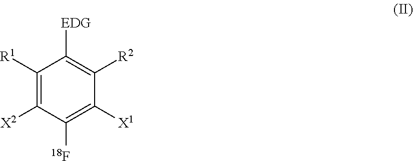

wherein EDG, R¹, R², X¹ and X² are as defined above.

The rearomatisation may exceptionally be performed on the isolated, purified compound of formula (IIa). Usually, however, said rearomatisation is performed in situ.

Thus, typically said rearomatisation comprises the addition (to the reaction mixture) of a reagent, which reagent effects cleavage of X³ from the carbon atom of the ring which is para to EDG′ in the compound of formula (IIa), to produce a compound of formula (II). Alternatively, the reagent may already be present in the reaction mixture. In some embodiments, for instance, the additive is the same reagent as that which effects rearomatisation. In particular, the acids described herein as additives may also act as effective rearomatisation reagents.

Any suitable reagent which effects cleavage of X³ from the carbon atom of the ring which is para to EDG′ in the compound of formula (IIa), and therefore rearomatisation, may be used. As the skilled person will appreciate, different reagents will be suitable for different groups X³, and the type of reagent employed will depend on the strength of the bond between X³ and the carbon atom of the ring which is para to EDG′. Typically, however, the reagent is an acid, base or oxidising agent.

Usually said cleavable surrogate group X³ is —CR¹⁸R¹⁹R²⁰, wherein R¹⁸ is H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, —O-acyl, acylamido or halo; and R¹⁹ and R²⁰, which are the same or different, are independently selected from unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀alkylamino, di(C₁₋₁₀)alkylamino, —O-acyl, acylamido or halo. More typically, said cleavable surrogate group X³ is —CR⁸R¹⁹R²⁰, wherein R¹⁸, R¹⁹ and R²⁰, which are the same or different, are independently selected from unsubstituted or substituted C₁₋₁₀- alkyl, unsubstituted or substituted C₃₋₁₀ cycloalkyl, and unsubstituted or substituted aryl. Thus, for instance, X³ may be tert-butyl.

For these particular groups, said rearomatisation usually comprises the addition of an acid. As the skilled person will appreciate, any suitable acid may be used. Typically, however, the acid is a mineral acid, a sulfonic acid or an organic acid. Particularly suitable are those which have a pKa less than or equal to the pKa of HF. Thus, the acid may be a mineral acid selected from H₂SO₄, HCl, HNO₃, HBr, HI and HClO₄; a sulfonic acid selected from camphorsulfonic acid (CSA), MeSO₃H and PhSO₃H; or an organic acid selected from p-nitrobenzoic acid and a halogenated organic acid.

Usually, the acid which is used for the rearomatisation is a strong organic acid, for instance p-nitrobenzoic acid or a halogenated organic acid. More typically, the acid used is a halogenated organic acid. Particularly preferred are halogenated organic acids having the formula R³¹—COOH, wherein R³¹ is a C₁₋₁₀ alkyl group substituted with one or more halo groups, for instance one, two or three halo groups, or wherein R³¹ is a C₁₋₁₀ perfluoroalkyl group. In one embodiment, the acid which is used for the rearomatisation is trifluoroacetic acid.

Once rearomatisation has been effected, the resulting compound of formula (II) may be recovered from the reaction mixture. Accordingly, the process of the invention may further comprise recovering the compound of formula (II). This compound can be recovered from the reaction mixture using standard methods for purification of ¹⁸F-labelled compounds, for instance by solid phase extraction and/or HPLC. Accordingly, in one embodiment the process further comprises purifying said compound of formula (II) by solid phase extraction and/or HPLC.

In one embodiment, R³ in the compound of formula (I) is said cleavable surrogate group X³, and one of X¹ and X² in the compound of formula (I) is a group of formula (Z2):

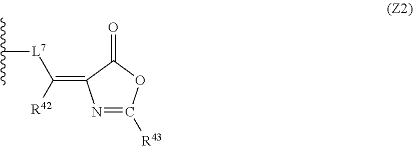

wherein L⁷, R⁴² and R⁴³ are as defined above;

and the process further comprises
(i) rearomatisation of the compound of formula (IIa), comprising cleavage of X³ from the ring carbon atom para to EDG′ in said compound; and
(ii) performing a reductive hydrolysis, in order to convert said group of formula (Z2) into a group of formula (Z3):

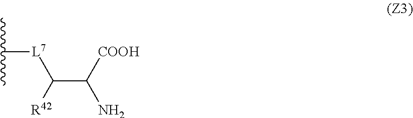

wherein L⁷ and R⁴² are as defined above for the group of formula (Z2);

thereby producing a compound of formula (IIZ)

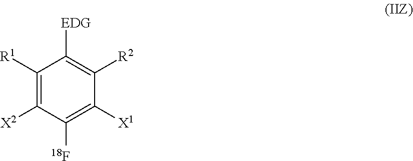

wherein EDG, R¹ and R² are as defined above, one of X¹ and X² is a said group of formula (Z3), and the other of X¹ and X² is as defined hereinbefore.

The rearomatisation of the compound of formula (IIa) may be performed as described above.

The step of performing a reductive hydrolysis typically comprises treatment (of the rearomatised compound produced in step (i)) with an acid and a reducing agent, typically in the presence of heat. The acid used for the reductive hydrolysis may be any suitable acid, for instance any of the acids described herein which can be used in the rearomatisation step or those which can be used as additives during the fluorination step. The acid may for instance be acetic acid. Any suitable reducing agent may be employed, for instance red phosphorus and HI may be used. Thus, the step of performing a reductive hydrolysis may comprise treatment (of the rearomatised compound produced in step (i)) with acetic acid, red phosphorus and HI, in the presence of heat. Typically the reaction mixture is heated to a temperature of up to about 130° C. in this step.

Typically, in this embodiment, L⁷ is a single bond, and R⁴² is H.

More typically, in this embodiment, EDG is OH, R¹ and R² are both H, L⁷ is a single bond, R⁴² is H, the other of X¹ and X² is H, and the compound of formula (IIZ) is as follows:

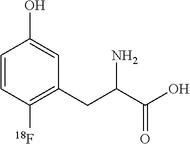 [SMILES: NC(Cc1cc(O)ccc1[18F])C(=O)O]

In another embodiment, R³ in the compound of formula (I) is said cleavable surrogate group X³, one of X¹ and X² in the compound of formula (I) is a group of formula (X2)

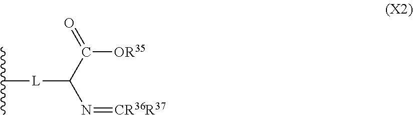

wherein R³⁵, R³⁶ and R³⁷ are as defined hereinbefore;

and the process further comprises
(i) rearomatisation of the compound of formula (IIa), comprising cleavage of X³ from the ring carbon atom para to EDG′ in said compound; and
(ii) a deprotection step, comprising converting said N═CR³⁶R³⁷ group in the group of formula (X2) into NH₂ and, when R³⁵ is a carboxyl protecting group, substituting H for said carboxyl protecting group, thereby converting the group of formula (X2) into a group of formula (X3):

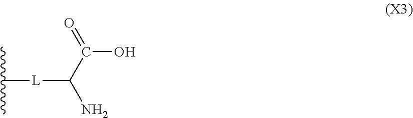

wherein L is as defined hereinbefore;

thereby producing a compound of formula (IIX)

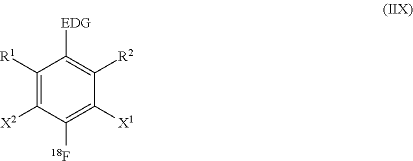

wherein EDG, R¹ and R² are as defined hereinbefore, one of X¹ and X² is a said group of formula (X3), and the other of X¹ and X² is as defined hereinbefore.

The rearomatisation of the compound of formula (IIa) may be performed as described above.

The deprotection step typically comprises treatment (of the rearomatised compound produced in step (i)) with an acid, usually in the presence of heat. The acid used for the deprotection step may be any suitable acid, for instance any of the acids described herein which can be used in the rearomatisation step or those which can be used as additives during the fluorination step. The acid may for instance be a mineral acid, such as hydrochloric acid. Thus, the deprotection step may comprise treatment (of the rearomatised compound produced in step (i)) with a mineral acid in the presence of heat. Typically the reaction mixture is heated to a temperature of up to about 110° C. in this step.

Typically, in this embodiment, L is CH₂.

Preferably, in this embodiment, the compound of formula (IIX) is as follows:

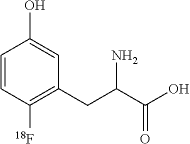 [SMILES: NC(Cc1cc(O)ccc1[18F])C(=O)O]

This embodiment may be used to produce enantioenriched products. Thus, typically, the compound of formula (IIX) is:

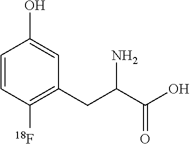 [SMILES: NC(Cc1cc(O)ccc1[18F])C(=O)O]

which is enantioenriched with the following enantiomer:

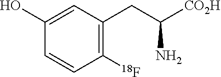 [SMILES: N[C@@H](Cc1cc(O)ccc1[18F])C(=O)O]

Typically, the enantiomeric excess of said enantiomer is at least 80%, more typically at least 95%.

Thus, typically, EDG is OH, R¹ and R² are both H, L is CH₂, the other of X¹ and X² is H, and the compound of formula (IIX) is as follows:

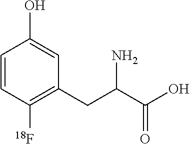 [SMILES: NC(Cc1cc(O)ccc1[18F])C(=O)O]

This embodiment may be used to produce enantioenriched products. Thus, in a preferred embodiment, EDG is OH, R¹ and R² are both H, L is CH₂, the other of X¹ and X² is H, and the compound of formula (IIX) is:

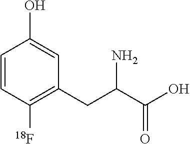 [SMILES: NC(Cc1cc(O)ccc1[18F])C(=O)O]

which is enantioenriched with the following enantiomer:

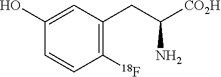 [SMILES: N[C@@H](Cc1cc(O)ccc1[18F])C(=O)O]

Typically, the enantiomeric excess of said enantiomer is at least 80%, more typically at least 95%.

When R³ in the compound of formula (I) is said bidentate cleavable surrogate group X⁴, the process typically further comprises rearomatisation of the compound of formula (IIc) or (IId) to produce a compound of formula (IIc′) or (IId′) respectively:

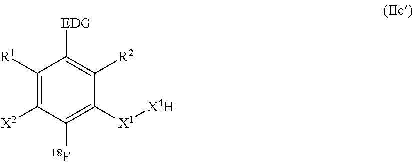

wherein EDG, R¹ and R² are as defined hereinbefore;

wherein X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2) as defined hereinbefore;

wherein X¹ is a C₁₋₂₀ alkyl, -L⁵-N(R⁴⁰)H, C₃₋₂₀ cycloalkyl, aryl, heteroaryl, C₃₋₁₀ heterocyclyl, C₁₋₂₀ alkoxy, C₁₋₁₀ alkylamino, di(C₁₋₁₀)alkylamino, acyl, amido or acylamido group, or a group of formula (X), formula (X2), formula (Y), formula (Z1) or formula (Z2), wherein X¹ is substituted with X⁴; and

wherein X⁴ is said bidentate cleavable surrogate group which is bonded (a) to X¹ and (b) to H;

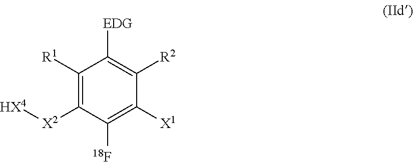

wherein EDG, R¹ and R² are as defined hereinbefore;

wherein X¹ is selected from H, unsubstituted or substituted -L⁵-N(R⁴⁰)H as defined hereinbefore, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2), as defined hereinbefore;

wherein X² is a C₁₋₂₀ alkyl, -L⁵-N(R⁴⁰)H as defined hereinbefore, C₃₋₂₀ cycloalkyl, aryl, heteroaryl, C₃₋₁₀ heterocyclyl, C₁₋₂₀ alkoxy, C₁₋₁₀ alkylamino, di(C₁₋₁₀)alkylamino, acyl, amido or acylamido group, or a group of formula (X), formula (X2), formula (Y), formula (Z1) or formula (Z2), as defined hereinbefore, wherein X² is substituted with X⁴; and

wherein X⁴ is said bidentate cleavable surrogate group which is bonded (a) to X² and (b) to H.

The rearomatisation may exceptionally be performed on the isolated, purified compound of formula (IIc) or (IId). Usually, however, said rearomatisation is performed in situ.

Thus, typically said rearomatisation comprises the addition (to the reaction mixture) of a reagent, which reagent effects cleavage of X⁴ from the carbon atom of the ring which is para to EDG′ in the compound of formula (IIc) or (IId), to produce a compound of formula (IIc′) or (IId′). Alternatively, the reagent may already be present in the reaction mixture. In some embodiments, for instance, the additive is the same reagent as that which effects rearomatisation. In particular, the acids described herein as additives may also act as effective rearomatisation reagents.

Any suitable reagent which effects cleavage of X⁴ from the carbon atom of the ring which is para to EDG′ in the compound of formula (IIc) or (IId), and which therefore effects rearomatisation of that compound, may be used. As the skilled person will appreciate, different reagents will be suitable for different groups X⁴, and the type of reagent employed will depend on the strength of the bond between X⁴ and the carbon atom of the ring which is para to EDG′. Typically, however, the reagent is an acid, base or oxidising agent.

Usually, said cleavable surrogate group X⁴ is *—C(R¹¹⁸)(R¹¹⁹)—X⁶—R¹²⁰—X⁷—**, wherein

* is the point of attachment of X⁴ to the ring carbon atom para to EDG′;

** is the point of attachment of X⁴ to X¹ or X²;

R¹¹⁸ is H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, —O-acyl, acylamido or halo;

R¹¹⁹ is unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, —O-acyl, acylamido or halo;

X⁶ is a bond, —O—, —N(R″)—, —O—C(O)— or —N(R″)C(O)—, wherein R″ is H, C₁₋₆ alkyl or aryl;

R¹²⁰ is a bond, optionally interrupted unsubstituted or substituted C₁₋₁₀-alkylene, C₁₋₁₀ perfluoroalkylene, unsubstituted or substituted arylene or perfluoroarylene; and

X⁷ is a bond, —O—, —N(R″)—, —O—C(O)—, —C(O)—O—, —N(R″)C(O)—, or —C(O)N(R″)— wherein R″ is H, C₁₋₆ alkyl or aryl.

Typically, R¹⁸ and R¹¹⁹, which are the same or different, are independently selected from unsubstituted or substituted C₁₋₁₀ alkyl, unsubstituted or substituted C₃₋₁₀ cycloalkyl, and unsubstituted or substituted aryl; and R¹²⁰ is a bond or unsubstituted or substituted C₁₋₆ alkylene. More typically, R¹⁸ and R¹¹⁹ are both methyl and R¹²⁰ is a bond or unsubstituted or substituted C₁₋₆ alkylene.

More typically, said cleavable surrogate group X⁴ is —C(R¹¹⁸)(R¹¹⁹)— wherein R¹¹⁸ and R¹¹⁹, which are the same or different, are independently selected from unsubstituted or substituted C₁₋₁₀ alkyl, unsubstituted or substituted C₃₋₁₀ cycloalkyl, and unsubstituted or substituted aryl. Preferably, R¹¹⁸ and R¹¹⁹ are both methyl, and the cleavable surrogate group X⁴ is C(CH₃)₂, i.e. dimethylmethylene.

For these particular groups, said rearomatisation usually comprises the addition of an acid. As the skilled person will appreciate, any suitable acid may be used. Typically, however, the acid is a mineral acid, a sulfonic acid or an organic acid. Particularly suitable are those which have a pKa less than or equal to the pKa of HF. Thus, the acid may be a mineral acid selected from H₂SO₄, HCl, HNO₃, HBr, HI and HClO₄; a sulfonic acid selected from camphorsulfonic acid (CSA), MeSO₃H and PhSO₃H; or an organic acid selected from p-nitrobenzoic acid and a halogenated organic acid.

Usually, the acid which is used for the rearomatisation is a strong organic acid, for instance p-nitrobenzoic acid or a halogenated organic acid. More typically, the acid used is a halogenated organic acid. Particularly preferred are halogenated organic acids having the formula R³¹—COOH, wherein R³¹ is a C₁₋₁₀ alkyl group substituted with one or more halo groups, for instance one, two or three halo groups, or wherein R³¹ is a C₁₋₁₀ perfluoroalkyl group. In one embodiment, the acid which is used for the rearomatisation is trifluoroacetic acid.

Once rearomatisation has been effected, the resulting compound of formula (IIc′) or (IId′) may be recovered from the reaction mixture. Accordingly, the process of the invention may further comprise recovering the compound of formula (IIc′) or (IId′). These compounds can be recovered from the reaction mixture using standard methods for purification of ¹⁸F-labelled compounds, for instance by solid phase extraction and/or HPLC. Accordingly, in one embodiment the process further comprises purifying said compound of formula (IIc′) or (IId′) by solid phase extraction and/or HPLC.

In one embodiment, R³ in the compound of formula (I) is said bidentate cleavable surrogate group, X⁴, and either:

(a) X¹ is a said group of formula -L⁵-N(R⁴⁰)H which is substituted with said bidentate cleavable surrogate group, X⁴, to form a group of formula *-L⁵-N(R⁴⁰)—X⁴⁴**, wherein * is the point of attachment of X¹ to the ring carbon atom meta to EDG or EDG′ and ** is the point of attachment of X⁴ to the ring carbon atom para to EDG or EDG′; or

(b) X² is a said group of formula -L⁵-N(R⁴⁰)H which is substituted with said bidentate cleavable surrogate group, X⁴, to form a group of formula *-L⁵-N(R⁴⁰)—X⁴-** wherein * is the point of attachment of X² to the ring carbon atom meta to EDG or EDG′ and ** is the point of attachment of X⁴ to the ring carbon atom para to EDG or EDG′;

and the process further comprises rearomatisation of the compound of formula (IIc) or (IId) to produce a compound of formula (IIc″) or (IId″) respectively:

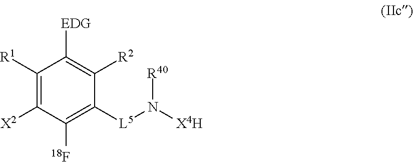

wherein EDG, R¹, R², L⁵ and R⁴⁰ are as defined in above;

wherein X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2) as defined above; and

wherein X⁴ is said bidentate cleavable surrogate group which is bonded (a) to X¹ and (b) to H;

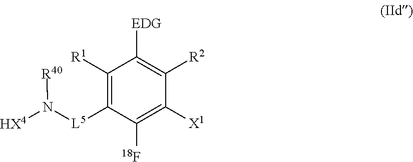

wherein EDG, R¹, R², L⁵ and R⁴⁰ are as defined above;

wherein X¹ is selected from H, unsubstituted or substituted -L⁵-N(R⁴⁰)H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2), as defined above; and

wherein X⁴ is said bidentate cleavable surrogate group which is bonded (a) to X² and (b) to H.

Typically, L⁵ is —CH₂—CH₂—, R⁴⁰ is benzyl and X⁴ is —C(CH₃)₂—.

Rearomatisation can be effected as described above. Once rearomatisation has been effected, the resulting compound of formula (IIc″) or (IId″) may be recovered from the reaction mixture. Accordingly, the process of the invention may further comprise recovering the compound of formula (IIc″) or (IId″). These compounds can be recovered from the reaction mixture using standard methods for purification of ¹⁸F-labelled compounds, for instance by solid phase extraction and/or HPLC. Accordingly, in one embodiment the process further comprises purifying said compound of formula (IIc″) or (IId″) by solid phase extraction and/or HPLC.

In another embodiment, R³ in the compound of formula (I) is said bidentate cleavable surrogate group, X⁴, and either:

(a) X¹ is a said group of formula (Z1) which is substituted with said bidentate cleavable surrogate group, X⁴, to form a group of formula (Z12)

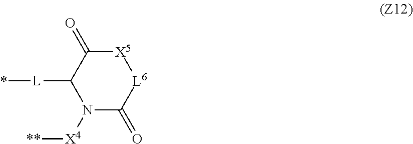

wherein * is the point of attachment of X¹ to the ring carbon atom meta to EDG or EDG′, ** is the point of attachment of X⁴ to the ring carbon atom para to EDG or EDG′ and X⁵, L⁶ and L are as defined above for (Z1); or

(b) X² is a said group of formula (Z1) which is substituted with said bidentate cleavable surrogate group, X⁴, to form a said group of formula (Z12)

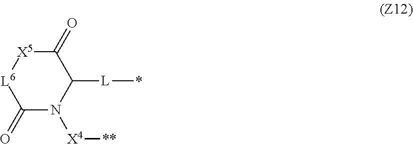

wherein * is the point of attachment of X² to the ring carbon atom meta to EDG or EDG′, ** is the point of attachment of X⁴ to the ring carbon atom para to EDG or EDG′, and X⁵, L⁶ and L are as defined above for (Z1);

and the process further comprises rearomatisation of the compound of formula (IIc) or (IId) to produce a compound of formula (IIc′″) or (IId′″) respectively:

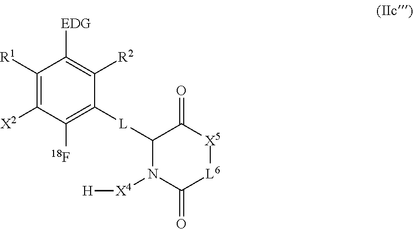

wherein EDG, R¹, R², L, X⁵ and L⁶ are as defined above;

wherein X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2) as defined above; and

wherein X⁴ is said bidentate cleavable surrogate group which is bonded (a) to X¹ and (b) to H;

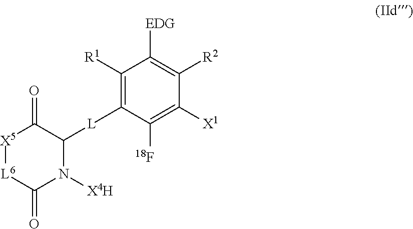

wherein EDG, R¹, R², L, X⁵ and L⁶ are as defined above;

wherein X¹ is selected from H, unsubstituted or substituted -L⁵-N(R⁴⁰)H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2), as defined above; and

wherein X⁴ is said bidentate cleavable surrogate group which is bonded (a) to X² and (b) to H.

Typically, L is CH₂; X⁵ is NR⁴⁴, wherein R⁴⁴ is unsubstituted C₁₋₆ alkyl; L⁶ is CH₂; and X⁴ is —C(CH₃)₂—.

Rearomatisation can be effected as described above. Once rearomatisation has been effected, the resulting compound of formula (IIc′″) or (IId′″) may be recovered from the reaction mixture. Accordingly, the process of the invention may further comprise recovering the compound of formula (IIc′″) or (IId′″). These compounds can be recovered from the reaction mixture using standard methods for purification of ¹⁸F-labelled compounds, for instance by solid phase extraction and/or HPLC. Accordingly, in one embodiment the process further comprises purifying said compound of formula (IIc′″) or (IId′″) by solid phase extraction and/or HPLC.

In another embodiment, when R³ in the compound of formula (I) is said bidentate cleavable surrogate group X⁴ the process further comprises:

(i) rearomatisation of said compound of formula (IIc) or (IId), comprising cleavage of X⁴ from the ring carbon atom para to EDG′ in said compound; and

(ii) cleavage of X⁴ from the group X¹ or X² to which X⁴ is bonded; thereby producing a compound of formula (II):

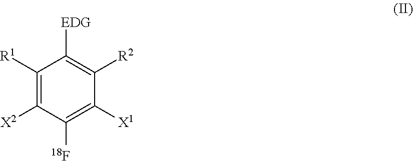

wherein EDG, R¹ and R² are as defined above; and

one of X¹ and X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, unsubstituted or substituted C₁₋₂₀ alkoxy, amino, unsubstituted or substituted C₁₋₁₀ alkylamino, unsubstituted or substituted di(C₁₋₁₀)alkylamino, unsubstituted or substituted acyl, unsubstituted or substituted amido, unsubstituted or substituted acylamido, halo, cyano and a group of formula (X), formula (X2), formula (Y), formula (Z1) or formula (Z2) as defined above; and

the other of X¹ and X² is selected from unsubstituted or substituted C₁₋₂₀ alkyl, unsubstituted or substituted -L⁵-N(R⁴⁰)H, unsubstituted or substituted C₃₋₂₀ cycloalkyl, unsubstituted or substituted aryl, unsubstituted or substituted heteroaryl, unsubstituted or substituted C₃₋₁₀ heterocyclyl, unsubstituted or substituted C₁₋₂₀ alkoxy, unsubstituted or substituted C₁₋₁₀ alkylamino, unsubstituted or substituted di(C₁₋₁₀)alkylamino, unsubstituted or substituted acyl, unsubstituted or substituted amido, unsubstituted or substituted acylamido, and a group of formula (X), formula (X2), formula (Y), formula (Z1) or formula (Z2) as defined above.

In this embodiment, the re-amortization step (i) may be performed as described above. The rearomatisation is usually performed in situ. Thus, typically said rearomatisation comprises the addition (to the reaction mixture) of a reagent, which reagent effects cleavage of X⁴ from the carbon atom of the ring which is para to EDG′ in the compound of formula (IIc) or (IId). Alternatively, the reagent may already be present in the reaction mixture.

Any suitable reagent which effects cleavage of X⁴ from the carbon atom of the ring which is para to EDG′ in the compound of formula (IIc) or (IId), and which therefore effects rearomatisation of that compound, may be used. As the skilled person will appreciate, different reagents will be suitable for different groups X⁴, and the type of reagent employed will depend on the strength of the bond between X⁴ and the carbon atom of the ring which is para to EDG′. Typically, however, the reagent is an acid, base or oxidising agent.

Usually, said cleavable surrogate group X⁴ is *—C(R¹¹⁸)(R¹¹⁹)—X⁶—R¹²⁰—X⁷—**, wherein

* is the point of attachment of X⁴ to the ring carbon atom para to EDG′;

** is the point of attachment of X⁴ to X¹ or X²;

R¹⁸ is H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, —O-acyl, acylamido or halo;

R¹¹⁹ is unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, —O-acyl, acylamido or halo;

X⁶ is a bond, —O—, —N(R″)—, —O—C(O)— or —N(R″)C(O)—, wherein R″ is H, C₁₋₆ alkyl or aryl;

R¹²⁰ is a bond, optionally interrupted unsubstituted or substituted C₁₋₁₀ alkylene, C₁₋₁₀ perfluoroalkylene, unsubstituted or substituted arylene or perfluoroarylene; and

X⁷ is a bond, —O—, —N(R″)—, —O—C(O)—, —C(O)—O—, —N(R″)C(O)—, or —C(O)N(R″)— wherein R″ is H, C₁₋₆ alkyl or aryl.

Typically, R¹¹⁸ and R¹¹¹⁹, which are the same or different, are independently selected from unsubstituted or substituted C₁₋₁₀ alkyl, unsubstituted or substituted C₃₋₁₀ cycloalkyl, and unsubstituted or substituted aryl; and R¹²⁰ is a bond or unsubstituted or substituted C₁₋₆ alkylene. More typically, R¹⁸ and R¹¹⁹ are both methyl and R¹²⁰ is a bond or unsubstituted or substituted C₁₋₆ alkylene.

More typically, said cleavable surrogate group X⁴ is —C(R¹¹⁸)(R¹¹⁹)— wherein R¹¹⁸ and R¹¹⁹, which are the same or different, are independently selected from unsubstituted or substituted C₁₋₁₀ alkyl, unsubstituted or substituted C₃₋₁₀ cycloalkyl, and unsubstituted or substituted aryl. Preferably, R¹¹¹⁸ and R¹¹⁹ are both methyl, and the cleavable surrogate group X⁴ is C(CH₃)₂.

For these particular groups, said rearomatisation in step (i) usually comprises the addition of an acid. As the skilled person will appreciate, any suitable acid may be used. Typically, however, the acid is a mineral acid, a sulfonic acid or an organic acid. Particularly suitable are those which have a pKa less than or equal to the pKa of HF. Thus, the acid may be a mineral acid selected from H₂SO₄, HCl, HNO₃, HBr, HI and HClO₄; a sulfonic acid selected from camphorsulfonic acid (CSA), MeSO₃H and PhSO₃H; or an organic acid selected from p-nitrobenzoic acid and a halogenated organic acid.

Usually, the acid which is used for the rearomatisation is a strong organic acid, for instance p-nitrobenzoic acid or a halogenated organic acid. More typically, the acid used is a halogenated organic acid. Particularly preferred are halogenated organic acids having the formula R³¹—COOH, wherein R³¹ is a C₁₋₁₀ alkyl group substituted with one or more halo groups, for instance one, two or three halo groups, or wherein R³¹ is a C₁₋₁₀ perfluoroalkyl group. In one embodiment, the acid which is used for the rearomatisation is trifluoroacetic acid.

Once rearomatisation has been effected in step (i), X⁴ is then cleaved from the group X¹ or X² to which X⁴ is bonded, in step (ii), to produce said compound of formula (II). Steps (i) and (ii) can be one and the same step, i.e. in some cases the reagent which is used for the rearomatisation may be suitable for cleaving X⁴ from the group X¹ or X². In other cases, however, a different reagent will need to be used. As the skilled person will appreciate, the reagent chosen depends on the type of linkage between X⁴ and the group X¹ or X².

Once cleavage of X⁴ from the group X¹ or X² has been effected, the resulting compound of formula (II) may be recovered from the reaction mixture. Accordingly, the process of the invention may further comprise recovering the compound of formula (II). This compound can be recovered from the reaction mixture using standard methods for purification of ¹⁸F-labelled compounds, for instance by solid phase extraction and/or HPLC. Accordingly, in one embodiment the process further comprises purifying said compound of formula (II) by solid phase extraction and/or HPLC.

In one embodiment, R³ in the compound of formula (I) is said bidentate cleavable surrogate group, X⁴, and either:

(a) X¹ is a said group of formula -L⁵-N(R⁴⁰)H which is substituted with said bidentate cleavable surrogate group, X⁴, to form a group of formula *-L⁵-N(R⁴⁰)—X⁴**, wherein * is the point of attachment of X¹ to the ring carbon atom meta to EDG or EDG′ and ** is the point of attachment of X⁴ to the ring carbon atom para to EDG or EDG′; or

(b) X² is a said group of formula -L⁵-N(R⁴⁰)H which is substituted with said bidentate cleavable surrogate group, X⁴, to form a group of formula *-L⁵-N(R⁴⁰)—X⁴-**, wherein * is the point of attachment of X² to the ring carbon atom meta to EDG or EDG′ and ** is the point of attachment of X⁴ to the ring carbon atom para to EDG or EDG′;

and the process further comprises:

(i) rearomatisation of said compound of formula (IIc) or (IId), comprising cleavage of X⁴ from the ring carbon atom para to EDG′ in said compound; and

(ii) cleavage of X⁴ from the group X¹ or X² to which X⁴ is bonded; thereby producing a compound of formula (IIc″″) or (IId″″) respectively:

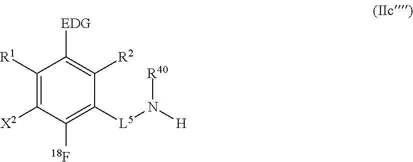

wherein EDG, R¹, R², L⁵ and R⁴⁰ are as defined above; and

X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2) as defined above;

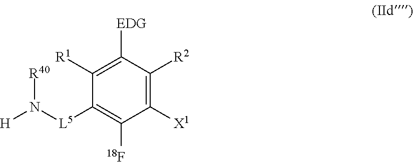

wherein EDG, R¹, R², L⁵ and R⁴⁰ are as defined above; and

X¹ is selected from H, unsubstituted or substituted -L⁵-N(R⁴⁰)H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2), as defined above.

Typically, L⁵ is —CH₂—CH₂—, R⁴⁰ is benzyl and X⁴ is —C(CH₃)₂—.

Rearomatisation can be effected as described above. Once rearomatisation has been effected in step (i), X⁴ is then cleaved from the group X¹ or X² to which X⁴ is bonded, in step (ii), to produce said compound of formula (IIc″″) or (IId″″). Steps (i) and (ii) can be one and the same step, i.e. in some cases the reagent which is used for the rearomatisation may be suitable for cleaving X⁴ from the group X¹ or X². In other cases, however, a different reagent will need to be used. As the skilled person will appreciate, the reagent chosen depends on the type of linkage between X⁴ and the group X¹ or X²

Once cleavage of X⁴ from the group X¹ or X² has been effected, the resulting compound of formula (IIc″″) or (IId″″) may be recovered from the reaction mixture.

Accordingly, the process of the invention may further comprise recovering the compound of formula (IIc″″) or (IId″″). These compound can be recovered from the reaction mixture using standard methods for purification of ¹⁸F-labelled compounds, for instance by solid phase extraction and/or HPLC. Accordingly, in one embodiment the process further comprises purifying said compound of formula (IIc″″) or (IId″″) by solid phase extraction and/or HPLC.

Once cleavage of X⁴ from the group X¹ or X² has been effected (whether or not the resulting compound is recovered from the reaction mixture) the process may further comprises a deprotection step, comprising substituting H for said amino protecting group R⁴⁰, thereby converting the group —NHR⁴⁰ in the compound of formula (IIc″″) or (IId″″) into a —NH₂ group. Suitable reaction conditions for deprotection are well known to the skilled person, and include nucleophilic substitution, catalytic hydrogenation and acid hydrolysis.

In one embodiment, R³ in the compound of formula (I) is said bidentate cleavable surrogate group, X⁴, and either:

(a) X¹ is a said group of formula (Z1) which is substituted with said bidentate cleavable surrogate group, X⁴, to form a group of formula (Z12)

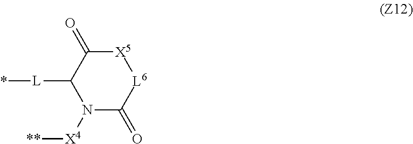

wherein * is the point of attachment of 12) to the ring carbon atom meta to EDG or wherein * is the point of attachment of X¹ to the ring carbon atom meta to EDG or EDG′ and ** is the point of attachment of X⁴ to the ring carbon atom para to EDG or EDG′; or

(b) X² is a said group of formula (Z1) which is substituted with said bidentate cleavable surrogate group, X⁴, to form a said group of formula (Z12)

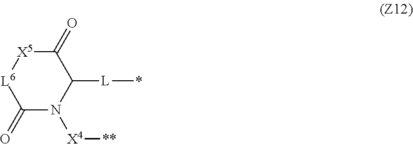

wherein * is the point of attachment of X² to the ring carbon atom meta to EDG or EDG′ and ** is the point of attachment of X⁴ to the ring carbon atom para to EDG or EDG′;

and the process further comprises

(i) rearomatisation of said compound of formula (IIc) or (IId), comprising cleavage of X⁴ from the ring carbon atom para to EDG′ in said compound; and

(ii) cleavage of X⁴ from the group X¹ or X² to which X⁴ is bonded; thereby producing a compound of formula (IIc′″″) or (IId′″″) respectively:

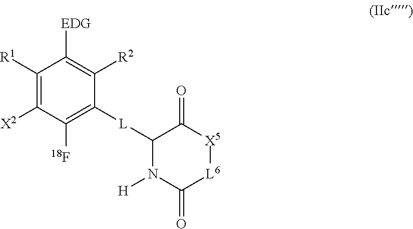

wherein EDG, R¹, R², L, X^{s} and L⁶ are as defined above; and

wherein X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2) as defined above;

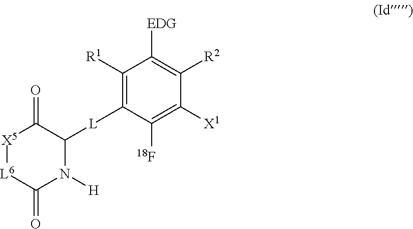

wherein EDG, R¹, R², L, X⁵ and L⁶ are as defined above; and

wherein X¹ is selected from H, unsubstituted or substituted -L⁵-N(R⁴⁰)H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2), as defined above.

Typically, L is CH₂; X⁵ is NR⁴⁴, wherein R⁴⁴ is unsubstituted C₁₋₆ alkyl; L⁶ is CH₂; and X⁴ is —C(CH₃)₂—.

Rearomatisation can be effected as described above. Once rearomatisation has been effected in step (i), X⁴ is then cleaved from the group X¹ or X² to which X⁴ is bonded, in step (ii), to produce said compound of formula (IIc′″″) or (IId′″″). Steps (i) and (ii) can be one and the same step, i.e. in some cases the reagent which is used for the rearomatisation may be suitable for cleaving X⁴ from the group X¹ or X². In other cases, however, a different reagent will need to be used. As the skilled person will appreciate, the reagent chosen depends on the type of linkage between X⁴ and the group X¹ or X².

Once cleavage of X⁴ from the group X¹ or X² has been effected, the resulting compound of formula (IIc′″″) or (IId″″) may be recovered from the reaction mixture. Accordingly, the process of the invention may further comprise recovering the compound of formula (IIc′″″) or (IId′″″). These compound can be recovered from the reaction mixture using standard methods for purification of ¹⁸F-labelled compounds, for instance by solid phase extraction and/or HPLC. Accordingly, in one embodiment the process further comprises purifying said compound of formula (IIc′″″) or (IId′″″) by solid phase extraction and/or HPLC.

Typically, however, the compound of formula (IIc′″″) or (IId′″″) (whether or not recovered from the reaction mixture) is also hydrolysed in order to cleave the X⁵-L⁶-C(O) moiety from the compound. Typically, this is achieved by acid hydrolysis, for instance by treatment with one or more acids at a temperature of up to about 120° C., or up to about 100° C. The acid may be any suitable acid, for instance any of the acids defined herein which can be used in the rearomatisation step or those which can be used as additives during the fluorination step. The acid hydrolysis is typically performed in situ.

Accordingly, the process typically further comprises a hydrolysis step, comprising hydrolysing the X⁵—C(O) bond and the N(H)—C(O) bond in the compound of formula (IIc′″″) or (IId′″″) in order to cleave the X⁵-L⁶-C(O) moiety from the compound, thereby producing a compound of formula (IIc″″″) or (IId″″″) respectively:

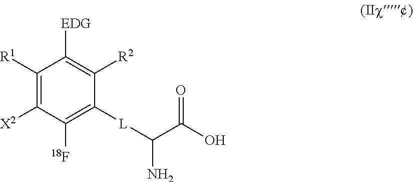

wherein EDG, R¹, R² and L are as defined above; and

X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2) as defined above;

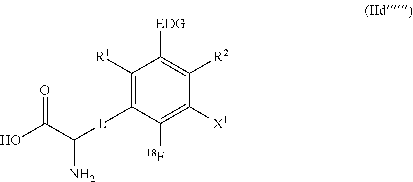

wherein EDG, R¹, R² and L are as defined above; and

wherein X¹ is selected from H, unsubstituted or substituted -L⁵-N(R⁴⁰)H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2), as defined above.

Typically, L is CH₂; X⁵ is NR⁴⁴, wherein R⁴⁴ is unsubstituted C₁₋₆ alkyl; L⁶ is CH₂; and X⁴ is —C(CH₃)₂—.

The resulting compound of formula (IIc″″″) or (IId″″″) may be recovered from the reaction mixture. Accordingly, the process of the invention may further comprise recovering the compound of formula (IIc″″″) or (IId″″″). These compound can be recovered from the reaction mixture using standard methods for purification of ¹⁸F-labelled compounds, for instance by solid phase extraction and/or HPLC. Accordingly, in one embodiment the process further comprises purifying said compound of formula (IIc″″″) or (IId″″″) by solid phase extraction and/or HPLC.

In one embodiment, R³ in the compound of formula (I) is said bidentate cleavable surrogate group, X⁴, and wherein either:

(a) X¹ is a said group of formula (Z1) which is substituted with said bidentate cleavable surrogate group, X⁴, to form a group of formula (Z12)

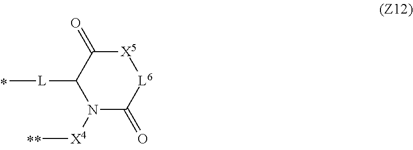

wherein * is the point of attachment of X¹ to the ring carbon atom meta to EDG or EDG′ and ** is the point of attachment of X⁴ to the ring carbon atom para to EDG or EDG′; or

(b) X² is a said group of formula (Z1) which is substituted with said bidentate cleavable surrogate group, X⁴, to form a said group of formula (Z12)

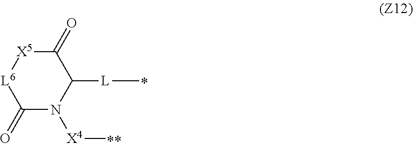

wherein * is the point of attachment of X to the ring carbon atom meta to EDG or EDG′ and ** is the point of attachment of X⁴ to the ring carbon atom para to EDG or EDG′;

and the process further comprises

(i) rearomatisation of said compound of formula (IIc) or (IId), comprising cleavage of X⁴ from the ring carbon atom para to EDG′ in said compound;

(ii) cleavage of X⁴ from the group X¹ or X² to which X⁴ is bonded; and

(iii) cleaving the X⁵-L⁶-C(O) moiety from the group X¹ or X² to which X⁴ is bonded, thereby producing a compound of formula (IIc″″″) or (IId″″″) respectively:

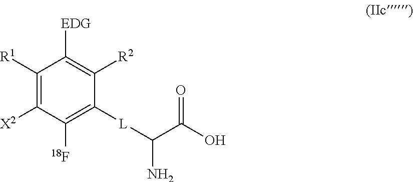

wherein EDG, R¹, R² and L are as defined above; and

X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2) as defined above;

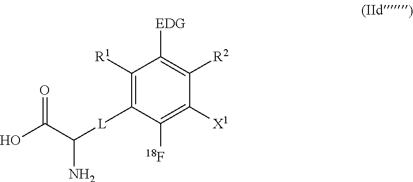

wherein EDG, R¹, R² and L are as defined above; and

wherein X¹ is selected from H, unsubstituted or substituted -L⁵-N(R⁴⁰)H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2), as defined above.

Typically, L is CH₂; X⁵ is NR⁴⁴, wherein R⁴⁴ is unsubstituted C₁₋₆ alkyl; L⁶ is CH₂; and X⁴ is —C(CH₃)₂—.

The step of cleaving the X⁵-L⁶-C(O) moiety from the group X¹ or X² to which X⁴ is bonded may comprise performing an acid hydrolysis. The acid hydrolysis may be performed in situ. Typically, performing an acid hydrolysis comprises treatment with one or more acids at a temperature of up to about 120° C., or up to about 100° C. The acid may be any suitable acid, for instance any of the acids defined herein which can be used in the rearomatisation step or those which can be used as additives during the fluorination step. The acid hydrolysis is typically performed in situ.

In some embodiments, R³ in the compound of formula (I) is H, and the step of treating said compound with [¹⁸F]fluoride in the presence of said oxidant produces a compound of formula (II) directly.

In the processes of the invention, EDG may be —NHR⁵ or —NR⁵⁵R⁵. When EDG is —NHR⁵, or —NR⁵⁵R⁵ the process may or may not further comprise a deprotection step comprising substituting H for R⁵, and when R⁵⁵ is present, substituting H for R⁵⁵, thereby producing a compound wherein EDG is —NH₂.

Thus, in some embodiments of the present invention, EDG is —NHR⁵ or —NR⁵⁵R⁵ and the process further comprises a deprotection step comprising substituting H for R⁵ in the compound of formula (II), and where R⁵⁵ is present, substituting H for R⁵⁵ in the compound of formula (II), thereby producing a compound of formula (IIb):

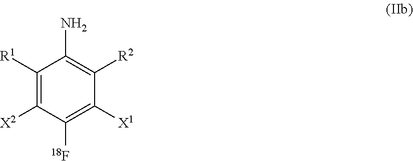

wherein

R¹ and R², which are the same or different, are independently selected from H, unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, acyl, amido, acylamido, halo, cyano, —OR¹⁰ and —NR¹¹R¹¹¹, wherein R¹⁰, R¹¹ and R¹¹¹ are as defined above; and

X¹ and X², which are the same or different, are independently selected from H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀o alkylamino, di(C₁₋₁₀)alkylamino, acyl, amido, acylamido, halo, cyano and a group of formula (X), formula (X2), formula (Y), formula (Z1) or formula (Z2) as defined above;

provided that X² and R¹ may together form a bidentate group such that R¹, X² and the ring carbon atoms to which R¹ and X² are bonded together form an unsubstituted or substituted fused aryl, heteroaryl, C₅₋₈ carbocyclic or C₅₋₈ heterocyclic ring;

and provided that X¹ and R² may together form a bidentate group such that R², X¹ and the ring carbon atoms to which R² and X¹ are bonded together form an unsubstituted or substituted fused aryl, heteroaryl, C₅₋₈ carbocyclic or C₅₋₈ heterocyclic ring.

The deprotection step may be performed on the isolated, purified compound of formula (II). Usually, however, said deprotection step is performed in situ.

Thus, the deprotection step may comprise the addition (to the reaction mixture) of a reagent, which reagent effects substitution of H for R⁵ (and, where R⁵⁵ is present, substitution of H for R⁵⁵) thereby producing a compound wherein EDG is —NH₂. Alternatively, however, the reagent may already be present in the reaction mixture. In some embodiments, for instance, the additive is the same reagent as that which effects substitution of H for R⁵ (and, where R⁵⁵ is present, substitution of H for R⁵⁵). In particular, the acids described herein as additives may also act as effective deprotection reagents which effect substitution of H for R⁵ (and, where R⁵⁵ is present, substitution of H for R⁵⁵).

Any suitable reagent which effects substitution of H for R⁵ (and, where R⁵⁵ is present, substitution of H for R⁵⁵) may be used. As the skilled person will appreciate, different reagents will be suitable for different groups R⁵, and the type of reagent employed will depend on the strength of the bond between R⁵ and N (and, where R⁵⁵ is present, the bond between R⁵⁵ and N). Typically, however, the reagent is an acid. In some embodiments, the acid is the same acid that is used as the additive in the fluorination reaction and is already therefore present in the reaction mixture.

Accordingly, said deprotection step typically requires the presence of an acid. In another embodiment, the deprotection step comprises the addition of an acid. Any suitable acid may be used. Typically, however, the acid is a mineral acid, a sulfonic acid or an organic acid. Particularly suitable are those which have a pKa less than or equal to the pKa of HF. Thus, the acid may be a mineral acid selected from H₂SO₄, HCl, HNO₃, HBr, HI and HClO₄; a sulfonic acid selected from camphorsulfonic acid (CSA), MeSO₃H and PhSO₃H; or an organic acid selected from p-nitrobenzoic acid and a halogenated organic acid.

Usually, the acid which is used for said deprotection step is a strong organic acid, for instance p-nitrobenzoic acid or a halogenated organic acid. More typically, the acid used is a halogenated organic acid. Particularly preferred are halogenated organic acids having the formula R³¹—COOH, wherein R³¹ is a C₁₋₁₀ alkyl group substituted with one or more halo groups, for instance one, two or three halo groups, or wherein R³¹ is a C₁₋₁₀ perfluoroalkyl group. In one embodiment, the acid which is used for said deprotection step is trifluoroacetic acid.

Once the deprotection step comprising substituting H for R⁵ (and, where R⁵⁵ is present, substituting H for R⁵⁵), and thereby producing a compound wherein EDG is —NH₂, has been effected, the resulting compound, which is typically of formula (IIb), may be recovered from the reaction mixture. Accordingly, the process of the invention may further comprise recovering the resulting compound, typically a compound of formula (IIb). This compound can be recovered from the reaction mixture using standard methods for purification of ¹⁸F-labelled compounds, for instance by solid phase extraction and/or HPLC. Accordingly, in one embodiment the process further comprises purifying said compound of formula (IIb) by solid phase extraction and/or HPLC.

In another embodiment, EDG in said compound of formula (I) is OH.

Typically, in the process of the invention for producing an ¹⁸F-labelled compound, R¹ and R², which are the same or different, are independently selected from H, unsubstituted or substituted C₁₋₂₀ alkyl, unsubstituted or substituted aryl, unsubstituted or substituted C₃₋₁₀ cycloalkyl, halo, —OR¹⁰ and —NR¹¹R¹¹¹;

wherein R¹⁰ is a hydroxyl protecting group; and

R¹¹ and R¹¹¹, which are the same or different, are independently selected from unsubstituted or substituted C₁₋₂₀ alkyl, unsubstituted or substituted C₃₋₁₀ cycloalkyl, C₁₋₂₀ unsubstituted or substituted aryl, —C(O)OR¹⁶ and —S(O)₂—R¹⁷, wherein R¹⁶ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl and 9-fluorenylmethyl; and wherein R¹⁷ is unsubstituted or substituted aryl or unsubstituted or substituted C₁₋₁₀ alkyl.

More typically, in the process of the invention, R¹ and R², which are the same or different, are independently selected from H, unsubstituted or substituted C₁₋₂₀ alkyl, unsubstituted or substituted aryl, halo and —OR¹⁰, wherein R¹⁰ is a hydroxyl protecting group.

Usually, R¹ and R², which are the same or different, are independently selected from H, unsubstituted or substituted C₁₋₁₀ alkyl, halo and —OR¹⁰, wherein R¹⁰ is a hydroxyl protecting group.

Suitable hydroxyl (OH) protecting groups are well known to the skilled person, and include, but are not limited to, acyl groups (for instance, acetyl, benzoyl and a group of formula (XX) below) and substituted or unsubstituted alkyl, alkenyl or alkaryl groups, for instance methoxymethyl (MOM), tetrahydropyranyl (THP), tert-butyl, benzyl, allyl, and tert-butyldimethylsilyl (TBDMS). Suitable reaction conditions for deprotection are also well known to the skilled person, and include hydrogenolysis and acid hydrolysis.

Particularly suitable hydroxyl protecting groups in this case are —CR¹²R¹³R¹⁴, C(O)R¹⁵, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, —C(O)OR¹⁶ and —S(O)₂R¹⁷;

wherein R¹² is H, unsubstituted or substituted C₁₋₁₀ alkyl, unsubstituted or substituted C₃₋₁₀ cycloalkyl, or unsubstituted or substituted aryl;

R¹³ and R¹⁴, which are the same or different, are independently selected from unsubstituted or substituted C₁₋₁₀ alkyl, unsubstituted or substituted C₃₋₁₀ cycloalkyl, and unsubstituted or substituted aryl;

R¹⁵ is selected from unsubstituted or substituted C₁₋₂₀ alkyl, unsubstituted or substituted C₃₋₁₀ cycloalkyl, unsubstituted or substituted C₃₋₁₀ heterocyclyl, unsubstituted or substituted aryl, unsubstituted or substituted heteroaryl, perfluoroaryl and a C₁₋₁₀ perfluoroalkyl group;

R¹⁶ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl and 9-fluorenylmethyl; and

R¹⁷ is unsubstituted or substituted aryl or unsubstituted or substituted C₁₋₁₀ alkyl.

Accordingly, in one embodiment, said hydroxyl protecting group R¹⁰ is selected from —CR¹²R¹³R¹⁴, —C(O)R¹⁵, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, —C(O)OR¹⁶ and —S(O)₂R¹⁷;

wherein R¹², R¹³, R¹⁴, R¹⁵, R¹⁶ and R¹⁷ are as defined above.

More typically, said hydroxyl protecting group R¹⁰ is selected from —CR¹²R³R¹⁴ and —C(O)R⁵, wherein:

R¹² is H, unsubstituted or substituted C₁₋₁₀ alkyl, unsubstituted or substituted C₃₋₁₀ cycloalkyl, or unsubstituted or substituted aryl;

R¹³ and R¹⁴, which are the same or different, are independently selected from unsubstituted or substituted C₁₋₁₀ alkyl, unsubstituted or substituted C₃₋₁₀ cycloalkyl, and unsubstituted or substituted aryl; and

R¹⁵ is selected from unsubstituted or substituted C₁₋₂₀ alkyl, unsubstituted or substituted C₃₋₁₀ cycloalkyl, unsubstituted or substituted C₃₋₁₀ heterocyclyl, unsubstituted or substituted aryl, unsubstituted or substituted heteroaryl, perfluoroaryl and a C₁₋₁₀ perfluoroalkyl group.

A particularly preferred hydroxyl protecting group for use in the present invention is a group of formula (XX):

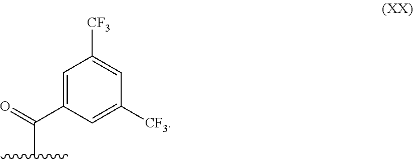 [SMILES: *C(=O)c1cc(C(F)(F)F)cc(C(F)(F)F)c1]

Typically, said hydroxyl protecting group R¹⁰ is said group of formula (XX).

Typically, when R¹ or R² is —OR¹⁰, wherein R¹⁰ is said hydroxyl protecting group, the process further comprises a deprotection step, performed after said step of treating said compound of formula (I) with said [¹⁸F]fluoride, said deprotection step comprising substituting H for R¹⁰ in said group —OR¹⁰, thereby converting said group —OR¹⁰ into an —OH group. Typically, the deprotection step comprises hydrogenolysis or acid hydrolysis. More typically, it comprises acid hydrolysis. Any suitable acid may be used. However, particularly preferred acids are those which are used in the rearomatisation step described above or those used as additives during the fluorination step as defined hereinbefore.

Accordingly, in one embodiment, at least one of R¹ and R² is —OR¹⁰, wherein R¹⁰ is said hydroxyl protecting group, and the process further comprises a deprotection step, performed after said step of treating said compound of formula (I) with said [¹⁸F]fluoride, said deprotection step comprising substituting H for R¹⁰ in said group —OR¹⁰, thereby converting said group —OR¹⁰ into an —OH group.

Typically, said deprotection step comprises the addition of an acid. Typically, the acid is any of those described herein which can be used in the rearomatisation step or those which can be used as additives during the fluorination step. The deprotection step may be performed in situ.

EDG may be OH or OR⁴, wherein R⁴ is as defined above. When EDG is OR⁴, the process typically further comprises a deprotection step, performed after said step of treating said compound of formula (I) with said [¹⁸F]fluoride, said deprotection step comprising substituting H for R⁴ in said group —OR⁴, thereby converting said group —OR⁴ into an —OH group. Typically, the deprotection step comprises hydrogenolysis or acid hydrolysis. More typically, it comprises acid hydrolysis. Any suitable acid may be used. However, particularly preferred acids are those which are used in the rearomatisation step described above or those used as additives during the fluorination step as defined hereinbefore. Typically, R⁴ is unsubstituted or substituted acyl, for instance a group of formula (XX):

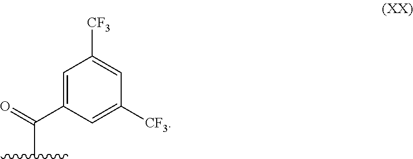 [SMILES: *C(=O)c1cc(C(F)(F)F)cc(C(F)(F)F)c1]

Typically, in the process of the invention for producing an 8F-labelled compound, X¹ and X², which are the same or different, are independently selected from H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, C₁₋₂₀ alkoxy, C₁₋₁₀ alkylamino, di(C₁₋₁₀)alkylamino, acyl, amido, acylamido, halo, cyano and a group of formula (X), formula (X2), formula (Y), formula (Z1) or formula (Z2) as defined above.

More typically, X¹ and X², which are the same or different, are independently selected from H, unsubstituted or substituted C₁₋₂₀ alkyl, unsubstituted or substituted C₃₋₂₀ cycloalkyl, C₁₋₁₀ perfluoroalkyl and a group of formula (X), formula (X2), formula (Y), formula (Z1) or formula (Z2) as defined hereinbefore.

More usually, X¹ and X², which are the same or different, are independently selected from H, unsubstituted or substituted C₁₋₂₀ alkyl, unsubstituted or substituted C₃₋₂₀ cycloalkyl, C₁₋₁₀ perfluoroalkyl and a group of formula (X), formula (X2), formula (Y), formula (Z1) or formula (Z2) as defined herein.

X¹ and X², which are the same or different, may be independently selected from H, unsubstituted or substituted C₁₋₂₀ alkyl, unsubstituted or substituted C₃₋₂₀ cycloalkyl, C₁₋₁₀ perfluoroalkyl and a group of formula (X), formula (X2), formula (Z1) or formula (Z2) as defined herein.

X¹ and X², which are the same or different, may be independently selected from H, unsubstituted or substituted C₁₋₂₀ alkyl, unsubstituted or substituted C₃₋₂₀ cycloalkyl, C₁₋₁₀ perfluoroalkyl and a group of formula (X), formula (X2) or formula (Z2) as defined herein.

X¹ and X², which are the same or different, may be independently selected from H, unsubstituted or substituted C₁₋₂₀ alkyl, unsubstituted or substituted C₃₋₂₀ cycloalkyl, C₁₋₁₀ perfluoroalkyl and a group of formula (X) or formula (X2) as defined herein.

X¹ and X², which are the same or different, may be independently selected from H, unsubstituted or substituted C₁₋₂₀ alkyl, unsubstituted or substituted C₃₋₂₀ cycloalkyl, C₁₋₁₀ perfluoroalkyl and a group of formula (X) as defined herein.

In one embodiment, R²² and R²³ in the group of formula (X), which are the same or different, are independently selected from H and an amino protecting group, which amino protecting group is selected from unsubstituted or substituted C₁₋₂₀ alkyl, unsubstituted or substituted C₃₋₁₀ cycloalkyl, C₁₋₂₀ perfluoroalkyl, acyl, unsubstituted or substituted aryl, perfluoroaryl, unsubstituted or substituted heteroaryl, unsubstituted or substituted C₃₋₁₀ heterocyclyl, —CHO, —C(O)OR²⁵ and —S(O)₂—R²⁶, wherein R²⁵ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R²⁶ is unsubstituted or substituted aryl or unsubstituted or substituted C₁₋₁₀ alkyl.

More typically, R²² and R²³ in the group of formula (X), which are the same or different, are independently selected from H and an amino protecting group, which amino protecting group is selected from unsubstituted or substituted C₁₋₁₀ alkyl, unsubstituted or substituted aryl, unsubstituted or substituted C₃₋₁₀ cycloalkyl, —CHO, —C(O)OR²⁵ and —S(O)₂—R²⁶, wherein R²⁵ is selected from unsubstituted or substituted C₁₋₁₀ alkyl, unsubstituted or substituted aryl, 9-fluorenylmethyl and pentafluorophenyl; and wherein R²⁶ is unsubstituted or substituted aryl or unsubstituted or substituted C₁₋₁₀ alkyl.

Suitable amino (NH₂) protecting groups are well known to the skilled person, and include, but are not limited to, t-Butyl carbamate (Boc), 9-fluorenylmethyl carbamate (Fmoc), benzyl carbamate, acyl groups, trityl, tosyl and benzyl. Typically, the amino protecting group is t-Butyl carbamate (Boc). Other amino protecting groups include C₁₋₂₀ alkyl and aryl groups. Suitable reaction conditions for deprotection are well known to the skilled person, and include nucleophilic substitution, catalytic hydrogenation and acid hydrolysis. Particularly suitable amino protecting groups in this case are unsubstituted or substituted C₁₋₂₀ alkyl, unsubstituted or substituted C₃₋₁₀ cycloalkyl, C₁₋₂₀ perfluoroalkyl, acyl, unsubstituted or substituted aryl, perfluoroaryl, unsubstituted or substituted heteroaryl, unsubstituted or substituted C₃₋₁₀ heterocyclyl, —C(O)OR¹⁶ and —S(O)₂R¹⁷, wherein R¹⁶ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R¹⁷ is unsubstituted or substituted aryl or unsubstituted or substituted C₁₋₁₀ alkyl.

Typically, R²⁴ in the group of formula (X) is H or a carboxyl protecting group, which carboxyl protecting group is unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl. Even more typically, R²⁴ in the group of formula (X) is H or a carboxyl protecting group, which carboxyl protecting group is unsubstituted or substituted C₁₋₁₀ alkyl, unsubstituted or substituted C₃₋₁₀ cycloalkyl, or unsubstituted or substituted aryl. In one embodiment R²⁴ is butyl, for instance tert-butyl.

Many suitable carboxyl (COOH) protecting groups are well known to the skilled person, and include, but are not limited to, unsubstituted or substituted C₁₋₆ alkyl (for instance methyl, ethyl and tert-butyl) and alkaryl (for instance benzyl); these protecting groups form simple esters to protect the carboxyl group. Suitable reaction conditions for deprotection of all such groups are also very well known to the skilled person, and include ester hydrolysis (saponification) and catalytic hydrogenation.

Typically, when X¹ or X² is a group of formula (X) in which at least one of R²² and R²³ is a said amino protecting group, the process further comprises a deprotection step, performed after said step of treating said compound of formula (I) with said [¹⁸F]fluoride, said deprotection step comprising substituting H for said amino protecting group or groups, thereby converting the group —NR²²R²³ in the group of formula (X) into an —NH₂ group. Typically, the deprotection step comprises nucleophilic substitution, catalytic hydrogenation or acid hydrolysis.

Accordingly, in one embodiment, at least one of X¹ and X² is a group of formula (X) in which at least one of R²² and R²³ is a said amino protecting group, and the process further comprises a deprotection step, performed after said step of treating said compound of formula (I) with said [¹⁸F]fluoride, said deprotection step comprising substituting H for said amino protecting group or groups, thereby converting the group —NR²²R²³ in the group of formula (X) into an —NH₂ group. Typically, the deprotection step comprises nucleophilic substitution, catalytic hydrogenation or acid hydrolysis.

Typically, when X¹ or X² is a group of formula (X) in which R²⁴ is a said carboxyl protecting group, the process further comprises a deprotection step, performed after said step of treating said compound of formula (I) with said [¹⁸F]fluoride, said deprotection step comprising substituting H for said carboxyl protecting group, thereby converting the group —COOR²⁴ in the group of formula (X) into a —COOH group. Typically, the deprotection step comprises ester hydrogenolysis or catalytic hydrogenation.

Accordingly, in one embodiment, at least one of X¹ and X² is a group of formula (X) in which R²⁴ is a said carboxyl protecting group, and the process further comprises a deprotection step, performed after said step of treating said compound of formula (I) with said [¹⁸F]fluoride, said deprotection step comprising substituting H for said carboxyl protecting group, thereby converting the group —COOR²⁴ in the group of formula (X) into a —COOH group. Typically, the deprotection step comprises ester hydrogenolysis or catalytic hydrogenation.

Said deprotection step or steps usually result in the conversion of said group of formula (X) into a group of formula (Xa) or (Xb)

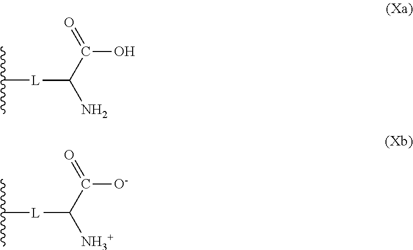

wherein L is unsubstituted or substituted C₁₋₄ alkylene.

Typically, in the process of the invention for producing an 18F-labelled compound, n in the group of formula (Y) is 1 and L⁴ in the group of formula (Y) is unsubstituted or substituted C₁₋₄ alkylene. Alternatively, n in the group of formula (Y) is 0 and L⁴ in the group of formula (Y) is a group of formula *═C(H)-alk-* * wherein * is the point of attachment of L⁴ to N, ** is the point of attachment of L⁴ to 0, and alk is unsubstituted or substituted C₁₋₃ alkylene.

Usually, when at least one of X¹ and X² is a group of formula (Y), the process of the invention further comprises a deprotection step, performed after said step of treating said compound of formula (I) with said [¹⁸F]fluoride, said deprotection step comprising converting said group of formula (Y) into a group of formula (Xa) or (Xb)

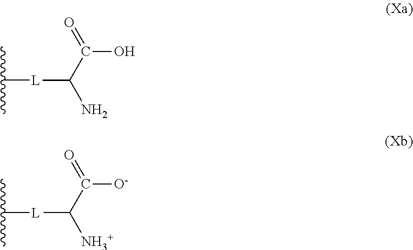

wherein L is unsubstituted or substituted C₁₋₄ alkylene.

Accordingly, in one embodiment, at least one of X¹ and X² is a group of formula (Y), and the process further comprises a deprotection step, performed after said step of treating said compound of formula (I) with said [¹⁸F]fluoride, said deprotection step comprising converting said group of formula (Y) into a group of formula (Xa) or (Xb)

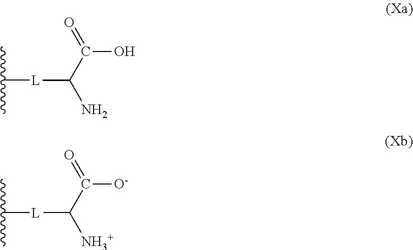

wherein L is unsubstituted or substituted C₁₋₄ alkylene.

This deprotection step typically comprises ester hydrogenolysis, catalytic hydrogenation, and/or acid hydrolysis.

Typically, after said deprotection step or steps described herein have been performed, the process of the invention further comprises recovering the resulting deprotected compound.

Such compounds can be recovered using standard methods for purification of ¹⁸F-labelled compounds, for instance by solid phase extraction and/or HPLC. Accordingly, in one embodiment the process further comprises purifying the resulting deprotected compound by solid phase extraction and/or by HPLC.

In one embodiment of the process of the invention, R¹, R², X¹ and X² are all H.

The present invention will be further illustrated in the Examples which follow:

### EXAMPLES

### Example 1

### Procedures for (A) Oxidative Nucleophilic ¹⁹F Fluorination, and (B) Oxidative Nucleophilic ¹⁸F Fluorination Using Conventional Apparatus and Using a Microfluidic Reactor

### A. Typical Procedure for Oxidative Nucleophilic ¹⁹F-Fluorination

To substrate (1 mmol) in solution of anhydrous CH₂Cl₂ (20 mL) at 0° C. or 25° C. was added HF-pyridine (4 mmol) followed by an oxidant (1 mmol). Reaction mixture was stirred for 10-2 h before quenching with K₂CO₃, filtered and concentrated in vacuo. The product was purified by column chromatography and analyzed by HPLC and/or ¹⁹F NMR.

### B. Typical Procedures for Oxidative Nucleophilic ¹⁸F-Fluorination

For Radiolabelling with Conventional Apparatus (e.g. Scintomics):

[¹⁸F]Fluoride was produced by the cyclotron of PETNET Solutions at Mont Vernon Hospital (UK) via the ¹⁸O(p,n)¹⁸F nuclear reaction and delivered as [¹⁸F]fluoride in [¹⁸O]H₂O (2-4 GBq, 1-3 mL). This target solution was passed through an anion exchange resin cartridge. [¹⁸F]Fluoride adsorbed on the charged-resin was eluted into a reaction vial with a solution of nBu₄NHCO₃ (8 mg) in 1 mL acetonitrile/water (4:1) or Cs₂CO₃ (8 mg) in water (500 μL). Excess water was removed under nitrogen stream at 120° C., and the resulting complex was azeotropically dried with acetonitrile (0.5 mL×3) under nitrogen stream. The resulting dry [¹⁸F]TBAF or [¹⁸F]CsF was further dissolved in the appropriate organic solvent and used for further reactions. Thin-layer chromatography (TLC) was carried out on aluminium plates coated with 60 F₂₅₄ silica and analyzed with a plastic scintillator/PMT detector, which usually gives the radiolabelling efficiency of the reaction. High performance liquid chromatography (HPLC) analysis was performed with the Gilson 322 system, equipped with a Na/PMT radiodetector and a UV-detector using the analytical Phenomenex Gemini-NX C18 column (150×4.6 mm, 5 μm).

To dried [¹⁸F]TBAF or [¹⁸F]CsF in CH₂Cl₂ at 25° C. was added the substrate (1 mmol) and trifluoroacetic acid (1.5% v/v) in CH₂Cl₂ followed by an oxidant (1 mmol) and the reaction mixture was stirred for 10 min. Trifluoroacetic acid (10% v/v) was added to the reaction mixture and stirred for a further 10 min at 25° C., should re-aromatization be necessary. Acetonitrile was added to dilute the reaction mixture. Reaction mixture was analyzed by HPLC and radio-TLC. Authenticity of the radiolabelled product was confirmed by spiking the reaction mixture with reference product.

For Radiolabelling with Microfluidic Apparatus (e.g. NanoTek®, Advion):

The radiosynthesis and azeotropic drying was automatically performed on a commercial microreactor device (NanoTek®, Advion). Cyclotron-produced non-carrier-added aqueous [¹⁸F]fluoride was first adsorbed onto an anion-exchange cartridge and subsequently released with 5001 solution of tBu₄NHCO₃ (4 mg) in acetonitrile/water (4:1) into the reactor. The solution was dried with two cycles of azeotropic drying with acetonitrile (300 μl) and dissolved in CH₂Cl₂/ClCH₂CH₂Cl (7:3 v/v, 1000 μl) containing the substrate (0.25-0.5 M). In a separate solution, the oxidant (0.25-0.5 M) was dissolved in CH₂Cl₂/ClCH₂CH₂Cl (7:3 v/v) containing trifluoroacetic acid (3%). Both solutions were delivered at various flow rate (2-30 μl/min) through the microfluidic reactor at 25° C. Chemical identity was verified with radio-HPLC using the Gilson 322 system, equipped with a NaI/PMT radiodetector and a UV-detector using the analytical Phenomenex Gemini-NX C18 column (150×4.6 mm, 5 km).

### Example 2

### Development of One-Pot Procedure to Synthesize 4-Fluorophenol from 4-Tert-Butylphenol and Phenol

The fluorination reaction (Scheme 1) was first validated with tert-butylphenol (3-99) using PhI(OAc)₂ as the oxidant and HF•py as the fluoride source (Table 1, Entry 1). In order to test its adaptability to ¹⁸F-radiochemistry, various fluoride sources were screened (Table 1). Interestingly, when TFA was added as an additive to either CsF or TBAF, the reaction proceed with 47% or 29% yield respectively (Table 1, Entry 2 and 4 respectively). Moreover, when PhI(TFA)₂ was used as the oxidant instead, the reaction is able to proceed without any additive, in 10% yield (Table 1, Entry 3).

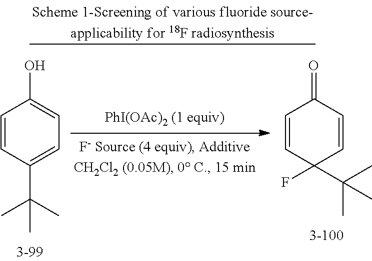

TABLE 1
Screening of various fluoride sources - results

| ENTRY^{a} | F⁻ SOURCE | ADDITIVE^{b} | YIELD^{c} |
|---|---|---|---|
| 1 | HF•py | None | 50% |
| 2 | CsF | TFA | 47% |
| 3^{e} | CsF | None | 10% |
| 4^{d} | TBAF | TFA | 29% |
| 5^{f} | HF•py | None | 21% |

(^{a}All reactions were performed on a 1 mmol scale.
^{b}The following amount of additive was added: TFA (4 equiv).
^{d}TBAF solution (1.0M in THF) or solid anhydrous TBAF was used.
^{e}PhI(TFA)₂ was used as oxidant instead.
^{f}Phenol (3-101) was used instead of tert-butyphenol.)

A one-pot procedure to synthesize 4-fluorophenol (3-93) from 4-tert-butylphenol (3-99) was developed (Scheme 2). Pleasingly, fluorination of 4-tert-butylphenol (3-99) followed by addition of 5% TFA gave the desired 4-fluorophenol (3-93) in 52% yield over the two steps.

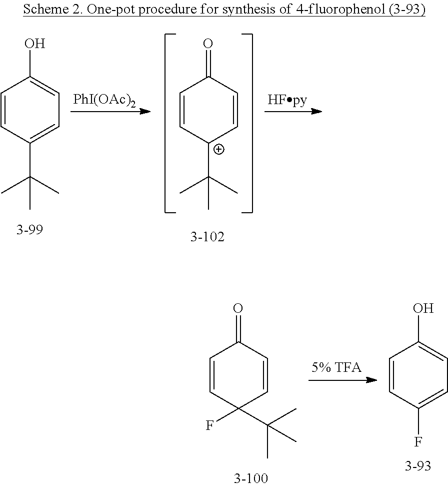

(Reagents and conditions: PhI(OAc)₂ (1 equiv), HF-py (4 equiv), CH₂Cl₂ (0.1 M), 0° C., 10 min; then TFA (5%), 15 min, 25° C., 52%.).

Alternatively, oxidative fluorination of unsubstituted substrate (i.e. no surrogate group in the para position) such as phenol is also feasible, albeit at a lower yield (21%) (Table 1, entry 5.)

### Example 3

### Development of One-Pot Procedure to Synthesize 4-Fluoroaniline

It was thought that the methodology might be extended to another prosthetic group 4-fluoroaniline (3-98), which is generally more useful for ¹⁸F-radiolabelling (Scheme 1). Pleasingly, N-tosylaniline (3-103a) was fluorinated under similar conditions, albeit in a lower yield (10%) (Table 2, Entry 1). Boc was also screened as it is more easily cleaved than a tosylate group. N-Boc-aniline (3-103c) reacts at room temperature over 30 min using PhI(TFA)₂ as the oxidant (Table 2, Entry 2, 3). With CsF as the fluoride source, N-Boc-aniline (3-103c) gave the desired product, albeit with a lower yield than using TBAF (Table 2, Entry 3).

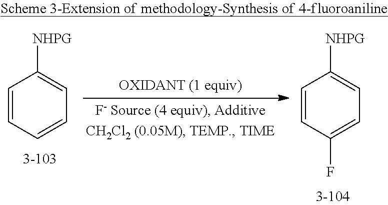

TABLE 2
Extension of methodology - Synthesis of 4-fluoroaniline

| ENTRY^{a} | PG | OXIDANT | F-SOURCE | ADDITIVE^{b} | TEMP. | TIME | YIELD^{c} |
|---|---|---|---|---|---|---|---|
| 1 | 3-103a: Ts | PhI(OAc)₂ | HF•py | None | 0° C. | 10 min | 10% |
| 2 | 3-103c: Boc | PhI(TFA)₂ | HF•py | None | 25° C. | 30 min | 49% |
| 3 | 3-103c: Boc | PhI(TFA)₂ | CsF | TFA | 25° C. | 30 min | 11% |

(^{a}All reactions are performed on 1 mmol scale. ^{b}4 equiv of additive was added. ^{c}Isolated yields.)

A one-pot procedure to synthesize 4-fluoroaniline (3-98) from N-Boc-aniline (3-103c) and N-Boc-4-tert-butylaniline (3-105c) was developed (Scheme 4). Thus, treatment of N-Boc-aniline (3-103c) and N-Boc-4-tert-butylaniline (3-105c) using the above standard fluorination conditions followed by the addition of 10% TFA gave the desired 4-fluoroaniline (3-98) in 15% and 37% yields over the two steps, respectively.

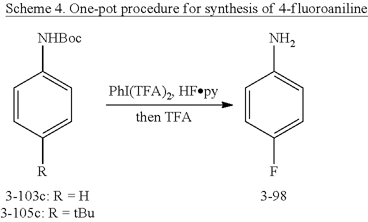

(Reagent and conditions. PhI(OAc)₂ (1 equiv), HF.py (4 equiv), CH2Cl2 (0.1 M), 25° C., min; then 10% TFA/CH₂Cl₂, 15 min, 25° C., 15% (from 3-103c), 37% (from 3-105c).)

### Example 4

### Radiosynthesis of 4-[¹⁸F]Fluorophenol ([¹⁸F]3-93) with Conventional Apparatus—Preliminary Results

Radiolabelling:

Radiolabelling work was performed at the Siemens-Oxford Medical Imaging Laboratory (SOMIL), Inorganic Chemistry Laboratory (ICL), Oxford using the Scintomics system behind lead shielding or carried out in a glove box with lead shielding. QMA and C₁₈ Sep-Pak cartridges were obtained from Waters (Milford, Mass.). [¹⁸F]Fluoride was produced by the cyclotron of PETNET Solutions at Mont Vernon Hospital (UK) via the ¹⁸O(p,n)¹⁸F nuclear reaction and delivered as [¹⁸F]fluoride in [18O]H₂O (2-4 GBq, 1-3 mL). This target solution was passed through the QMA anion exchange resin cartridge. [¹⁸F]Fluoride adsorbed on the charged-resin was eluted into a reaction vial with a solution of Kryptofix-222 (15 mg) and K₂CO₃ (3 mg) in 1 mL acetonitrile/water (4:1). Excess water was removed under N₂ stream at 120° C., and the resulting complex was azeotropically dried with acetonitrile (0.5 mL×3) under N₂ stream. The resulting dry complex of K[¹⁸F]F/Kryptofix-222 was further dissolved in the appropriate organic solvent and used for further reactions. Thin-layer chromatography (TLC) was carried out on aluminium plates coated with 60 F₂₅₄ silica and analyzed with a plastic scintillator/PMT detector, which usually gives the radiolabelling efficiency of the reaction. High performance liquid chromatography (HPLC) analysis was performed with the Gilson 322 system, equipped with a NaI/PMT radiodetector and a UV-detector using the analytical Phenomenex Gemini-NX C18 column (150×4.6 mm, 5 μm).

Synthesis from 4-tert-butylphenol

To dried TBA[¹⁸F]F or Cs[¹⁸F]F in CH₂Cl₂ (100 μL) at 25° C. was added 4-tert-butylphenol (3-99) (150 mg) and TFA (3 μL) in CH₂Cl₂ (100 μL) followed by PhI(OAc)₂ (320 mg) and the reaction mixture was stirred for 10 min. More TFA (20 μL) was added and the reaction mixture was stirred for a further 10 min at 25° C. MeCN (300 μL) was added to dilute the reaction mixture. Reaction mixture was analyzed by HPLC and radio-TLC. Authenticity of the radiolabelled product was confirmed by spiking the reaction mixture with reference product. [¹⁸F]3-93: RCY=4-6% (n=2) (based on radio-HPLC intergration); HPLC (gradient 5→95% B over 10 min; flow rate: 1 mL/min): t_{R}=6.27 min.

Synthesis from Phenol

Alternatively, to dried TBA[¹⁸F]F in CH₂Cl₂ (100 μL) at 25° C. was added phenol (3-101) (94 mg) and TFA (3 μL) in CH₂C₂ (100 μL) followed by PhI(OAc)₂ (320 mg) and the reaction mixture was stirred for 10 min. MeCN (300 μL) was added to quench the reaction. Reaction mixture was analyzed by HPLC and radio-TLC. Authenticity of the radiolabelled product was confirmed by spiking the reaction mixture with reference product. [¹⁸F]13-93: RCY=3% (based on radio-HPLC integration).

### Example 5

### Radiosynthesis of 4-[¹⁸F]Fluorophenol ([¹⁸F]3-93) in a Microfluidic Reactor—Results

Using the procedure described in Example 1 for radiolabelling in a microfluidic apparatus, 4-[¹⁸F]fluorophenol was synthesised from 4-tert-butylphenol with a radiochemical yield (RCY) of 94% and an isolated yield of 34%.

Using the same procedure, 4-[¹⁸F]fluorophenol was synthesised from phenol with a radiochemical yield (RCY) of 34% and an isolated yield of 11%.

### Example 6

### Tetrahydroisoquinoline

### Preparation and Fluorination

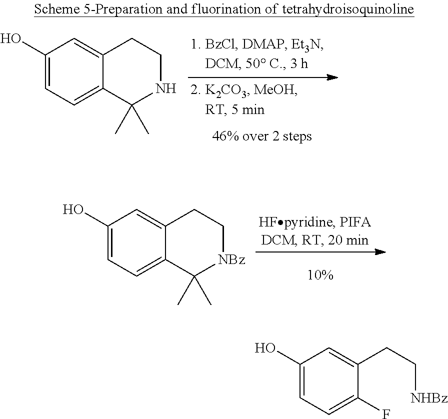

### (6-Hydroxy-1,1-dimethyl-3,4-dihydroisoquinolin-2(1H)-yl)(phenyl)methanone

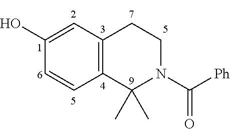 [SMILES: CC1(C)c2ccc(O)cc2CCN1C(=O)c1ccccc1]

Benzoyl chloride (591 μL, 5.1 mmol) was slowly added to a solution of 1,1-dimethyl-1,2,3,4-tetrahydroisoquinolin-6-ol (361 mg, 2.0 mmol), DMAP (5 mg, 0.04 mmol) and Et₃N (1.42 mL, 10.2 mmol) in DCM (30 mL) at RT. The reaction mixture was stirred at 50° C. for 3 h. The resulting mixture was diluted with DCM (70 mL), washed with H₂O (50 mL×2) and re-extracted with DCM (100 mL). The combined organic extracts were dried over anhydrous Na₂SO₄ and concentrated under reduced pressure. K₂CO₃ (0.94 g, 6.8 mmol) was added to a solution of the crude mixture in MeOH (30 mL) and stirred for 5 min at RT. The resulting mixture was diluted with H₂O (60 mL), acidified to pH 6-7 with 2 M HCl, and extracted with EtOAc (60 mL×2) and washed with H₂O (60 mL). The combined organic extracts were dried over anhydrous Na₂SO₄ and concentrated under reduced pressure. The crude product was purified by f.c.c. (eluent: DCM/Et₂O=94/6), yield=263 mg (46%) as a white solid. m.p.=211-214° C. R_{f} (DCM/Et₂O=94/6): 0.22. ¹H NMR (400 MHz, DMSO-d₆) δ: 9.27 (br. s, 1H, OH), 7.48-7.39 (m, 5H, Ph-H), 7.20 (d, J=8.8 Hz, 1H, H5), 6.67 (dd, J=2.5, 8.6 Hz, 1H, H6), 6.48 (d, J=2.5 Hz, 1H, H2), 3.39-3.30 (m, 2H, H8), 2.75-2.69 (m, 2H, H7), 1.79 (s, 6H, C(CH₃)₂). ¹³C NMR (101 MHz, DMSO-d₆) δ: 171.4 (C═O), 155.0 (C1), 138.9 (Ar—H), 135.2 (Ar—H), 134.9 (Ar—H), 129.4 (Ar—H), 128.5 (Ar—H), 127.4 (C5), 126.5 (Ar—H), 114.2 (C6), 114.0 (C2), 59.2 (C9), 44.9 (C8), 30.2 (C7), 27.5 (C(CH₃)₂). IR (Neat): 3307, 1608, 1593, 1413, 1227, 933, 864, 788, 693. HRMS (ESI⁺, m/z): calculated for C₁₈H₂₀NO₂ ([M+H]⁺) 282.1489. Found 282.1492.

HF•pyridine (70%, 26 μL, 1 mmol) was added to a solution of (6-Hydroxy-1,1-dimethyl-3,4-dihydroisoquinolin-2(1H)-yl)(phenyl)methanone (0.25 mmol) in anhydrous DCM (5 mL) at RT. [Bis(trifluoroacetoxy)iodo]benzene (107 mg, 0.25 mmol) was added to the reaction mixture 1 min later. The reaction mixture was stirred for 20 min at RT, and then concentrated under reduced pressure. The residue was dissolved in MeOD-d₄ and subjected to ¹⁹F NMR. Yield of the product (10%) was calculated based on ¹⁹F NMR peak intergration at 6-133 ppm with the addition of 2,4-dibromofluorobenzene in MeOD-d₄ (0.25 M, 100 μL, 0.25 mmol, δ-110 ppm) to the NMR sample as the internal reference.

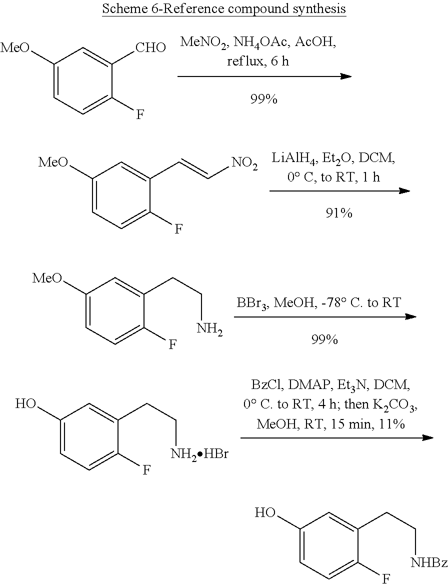

### Example 7

### Tetrahydroisoquinoline—a Tricyclic Precursor to 6-Fluoro-Meta-Tyrosine

### Preparation

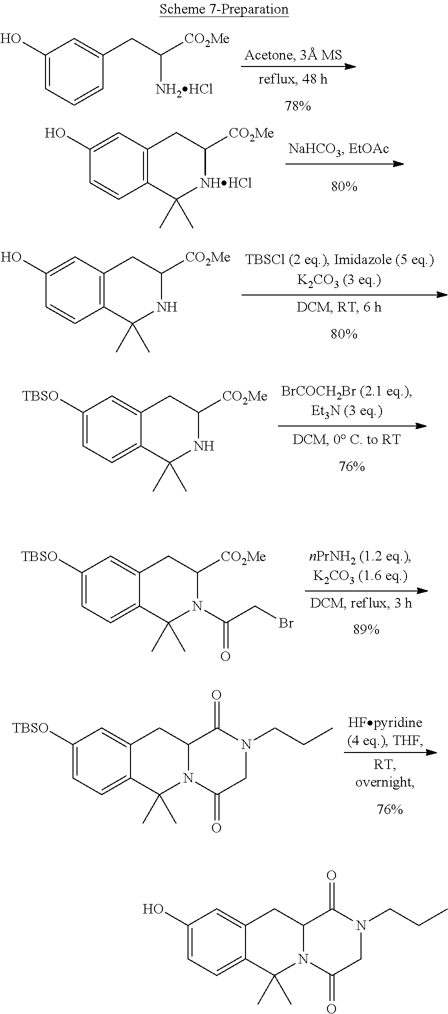

### Methyl 6-hydroxy-1,1-dimethyl-1,2,3,4-tetrahydroisoquinoline-3-carboxylate hydrochloride

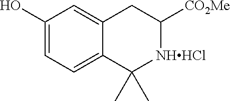

Methyl 3-hydroxyphenylalaninate hydrogen chloride (9.0 g, 38.8 mmol) was dispensed in acetone (100 mL) and refluxed with activated 3 Å molecular sieves in a Soxhlet extractor for 48 h until ¹H NMR showed full conversion of the starting material to product. The reaction mixture was cooled down, filtered and washed with acetone (40 mL) and Et₂O (40 mL×2), yield=9.38 g (89%) as a white solid. m.p.=238-240° C. ¹H NMR (400 MHz, D₂O) δ: 7.29 (d, J=8.8 Hz, 1H, Ar—H), 6.86 (dd, J=2.5, 8.6 Hz, 1H, Ar—H), 6.72 (d, J=2.5 Hz, 1H, Ar—H), 4.61 (dd, J=5.1, 12.6 Hz, 1H, CH₂CH), 3.91 (s, 3H, CO₂CH₃), 3.39 (dd, J=5.1, 17.4 Hz, 1H, CH₂CH), 3.21 (dd, J=12.6, 17.2 Hz, 1H, CH₂CH), 1.81 (s, 3H, C(CH₃)₂), 1.65 (s, 3H, C(CH₃)₂). ¹³C NMR (101 MHz, D₂O) δ: 170.3 (C═O), 155.8 (Ar—C), 130.9 (Ar—C), 129.5 (Ar—C), 127.5 (Ar—C), 116.1 (Ar—C), 115.3 (Ar—C), 59.7 (C(CH₃)₂), 54.4 (CH₂CH), 51.3 (CO₂CH₃), 29.2 (CH₂), 28.3 (CH₃), 27.6 (CH₃). IR (Neat): 3216, 1757, 1615, 1227, 894, 860, 819. HRMS (ESI⁺, m/z): calculated for C₁₃H₁₈NO₃ ([M+H]⁺) 236.1281. Found 236.1275.
Note: ¹³C spectrum is reported relative to 1,4-dioxane (δ: 67.19) as the internal reference.

### Methyl 6-{[tert-butyl(dimethyl)silyl]oxy}-1,1-dimethyl-1,2,3,4-tetrahydroisoquinoline-3-carboxylate

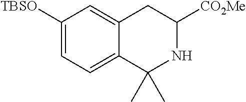 [SMILES: COC(=O)C1Cc2cc(O[Si](C)(C)C(C)(C)C)ccc2C(C)(C)N1]

tert-butyl(chloro)dimethylsilane (4.4 g, 29 mmol) was added to a solution of Methyl 6-hydroxy-1,1-dimethyl-1,2,3,4-tetrahydroisoquinoline-3-carboxylate hydrochloride (4 g, 15 mmol), K₂CO₃ (6.1 g, 44 mmol) and imidazole (5 g, 74 mmol) in DCM (100 mL) at RT. The reaction was stirred for 6 h at RT. The reaction mixture was diluted with H₂O (100 mL) and extracted with DCM (150 mL×2) and washed with H₂O (100 mL). The combined organic extracts were dried over anhydrous Na₂SO₄ and concentrated under reduced pressure. The crude product was purified by f.c.c. (eluent: hexane/EtOAc=4/1), yield=4.1 g (80%) as a pale yellow oil. R_{f} (hexane/EtOAc=4/1)=0.21. ¹H NMR (400 MHz, CDCl₃) δ: 7.06 (d, J=8.4 Hz, 1H, Ar—H), 6.66 (dd, J=2.6, 8.4 Hz, 1H, Ar—H), 6.54 (d, J=2.2 Hz, 1H, Ar—H), 3.88 (dd, J=4.1, 11.4 Hz, 1H, CH₂CH), 3.80 (s, 3H, CO₂CH₃), 2.97 (dd, J=4.1, 16.0 Hz, 1H, CH₂CH), 2.86 (dd, J=11.6, 16.0 Hz, 1H, CH₁CH), 1.50 (s, 3H, C(CH₃)₂), 1.41 (s, 3H, C(CH₃)₂), 0.97 (s, 9H, Si—C(CH₃)₃), 0.19 ppm (s, 6H, Si(CH₃)₂). ¹³C NMR (126 MHz, CDCl₃): 173.8 (C═O), 153.4 (Ar—C), 136.0 (Ar—C), 133.6 (Ar—C), 126.6 (Ar—C), 119.6 (Ar—C), 118.4 (Ar—C), 53.5 (C(CH₃)₂), 52.2 (CO₂CH₃), 51.9 (CH₂CH), 33.5 (CH₂CH), 31.9 (C(CH₃)₂), 31.0 (C(CH₃)₂), 25.6 (C(CH₃)₃), 18.1 (Si—C(CH₃)₃), −4.4 (Si(CH₃)₂). IR (DCM): 2956, 1744, 1608, 1253, 851, 780. HRMS (ESI⁺, m/z): calculated for C₁₉H₃₂NO₃Si ([M+H]⁺) 350.2146. Found 350.2140.

### Methyl 2-(bromoacetyl)-6-{[tert-butyl(dimethyl)silyl]oxy}-1,1-dimethyl-1,2,3,4-tetrahydroisoquinoline-3-carboxylate

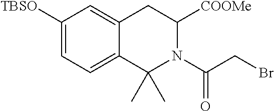 [SMILES: COC(=O)C1Cc2cc(O[Si](C)(C)C(C)(C)C)ccc2C(C)(C)N1C(=O)CBr]

2-bromoacetyl bromide (1.2 mL, 23 mmol) was added to a solution of Methyl 6-{[tert-butyl(dimethyl)silyl]oxy}-1,1-dimethyl-1,2,3,4-tetrahydroisoquinoline-3-carboxylate (4 g, 11 mmol) and Et₃N (4.8 mL, 34 mmol) in DCM (100 mL) at 0° C. The reaction was allowed to reach RT and stirred for 4 h. The reaction mixture was quenched by saturated NaHCO₃ solution (150 mL) and extracted with DCM (150 mL×2), washed with saturated NaHCO₃ solution (150 mL). The combined organic extracts were dried over anhydrous Na₂SO₄ and concentrated under reduced pressure. The crude product was purified by f.c.c. (eluent: hexane/EtOAc=4/1), yield=4 g (75%) as a red oil. R_{f} (hexane/EtOAc=4/1)=0.3. ¹H NMR (400 MHz, CDCl₃) δ: 7.17 (d, J=8.8 Hz, 1H, Ar—H, 6.72 (dd, J=2.5, 8.6 Hz, 1H, Ar—H), 6.59 (d, J=2.5 Hz, 1H, Ar—H), 4.92-4.85 (m, 1H, CH₂CH), 3.93 (d, J=10.6 Hz, 1H, CH₂Br), 3.84 (d, J=10.9 Hz, 1H, CH₂Br), 3.54 (s, 3H, CO₂CH₃), 3.27 (dd, J=2.8, 15.4 Hz, 1H, CH₂CH), 3.17 (dd, J=4.5, 15.2 Hz, 1H, CH₂CH), 1.97 (s, 3H, C(CH₃)₂), 1.67 (s, 3H, C(CH₃)₂), 0.98 (s, 9H, Si—C(CH₃)₃), 0.20 (s, 3H, Si(CH₃)₂), 0.19 (s, 3H, Si(CH₃)₂). ¹³C NMR (100 MHz, CDCl₃) δ: 170.7 (C═O), 166.4 (C═O), 153.8 (Ar—C), 136.6 (Ar—C), 131.8 (Ar—C), 126.3 (Ar—C), 119.4 (Ar—C), 119.1 (Ar—C), 61.0 (C(CH₃)₂), 57.4 (CH₂CH), 52.5 (CO₂CH₃), 32.0 (CH₂CH), 30.8 (C(CH₃)₂), 30.0 (CH₂Br), 25.6 (Si—C(CH₃)₃), 24.1 (C(CH₃)₂), 18.2 (C(CH₃)₃), −4.4 (Si(CH₃)₂). IR (DCM): 1740, 1657, 1260, 841, 782. HRMS (ESI⁺, m/z): calculated for C₂₁H₃₂BrNNaO₄Si ([M+Na]⁺) 492.1176. Found 492.1190.

### 9-{[tert-Butyl(dimethyl)silyl]oxy}-6,6-dimethyl-2-propyl-11,11a-dihydro-2H-pyrazino[1,2-b]isoquinoline-1, 4(3H, 6H)-dione

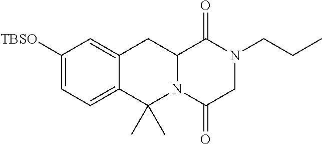 [SMILES: CCCN1CC(=O)N2C(Cc3cc(O[Si](C)(C)C(C)(C)C)ccc3C2(C)C)C1=O]

Methyl 2-(bromoacetyl)-6-{[tert-butyl(dimethyl)silyl]oxy}-1,1-dimethyl-1,2,3,4-tetrahydroisoquinoline-3-carboxylate (4 g, 8.6 mmol) was dissolved in a solution of DCM (20 mL) with 1-propylamine (847 □L, 10 mmol) and K₂CO₃ (1.9 g, 14 mmol). The reaction was refluxed for 3 h and cooled down. The reaction mixture was diluted with DCM (100 mL), washed with brine (150 mL×2) and re-extracted with DCM (150 mL). The combined organic extracts were dried over anhydrous Na₂SO₄ and concentrated under reduced pressure. The crude product was purified by f.c.c. (eluent: hexane/EtOAc=2/1), yield=3.2 g (89%) as a pale yellow oil. R_{f} (hexane/EtOAc=2/1)=0.3. ¹H NMR (400 MHz, CDCl₃) δ: 7.12 (d, J=8.6 Hz, 1H, Ar—H), 6.75 (dd, J=2.7, 8.7 Hz, 1H, Ar—H), 6.61 (d, J=2.5 Hz, 1H, Ar—H), 4.04 (dd, J=1.5, 17.4 Hz, 1H, COCH₂), 4.02 (dd, J=1.8, 11.1 Hz, 1H, CH₂CH), 3.93 (dd, J=1.3, 16.9 Hz, 1H, COCH₂), 3.44 (dd, J=2.0, 15.4 Hz, 1H, CH₂CH), 3.52-3.40 (m, 1H, NCH₂CH₂CH₃), 3.36-3.28 (m, 1H, NCH₂CH₂CH₃), 2.94 (dd, J=11.9, 15.4 Hz, 1H, CH₂CH), 1.87 (s, 3H, C(CH₃)₂), 1.86 (s, 3H, C(CH₃)₂), 1.67-1.60 (m, 2H, NCH₂CH₂CH₃), 0.99 (s, 9H, Si—C(CH₃)₃), 0.96 (t, J=7.6 Hz, 3H; NCH₂CH₂CH₃), 0.21 ppm (s, 6H, Si(CH₃)₂). ¹³C NMR (100 MHz, CDCl₃) δ: 164.5 (C═O), 162.3 (C═O), 153.6 (Ar—C), 135.9 (Ar—C), 134.0 (Ar—C), 126.8 (Ar—C), 119.3 (Ar—C), 118.8 (Ar—C), 62.5 (C(CH₃)₂), 55.9 (CH₂CH), 50.5 (COCH₂), 47.4 (NCH₂CH₂CH₃), 35.9 (CH₂CH), 31.8 (C(CH₃)₂), 25.6 (SiC(CH₃)₃), 24.8 (C(CH₃)₂), 19.6 (NCH₂CH₂CH₃), 18.1 (Si—C(CH₃)₃), 11.2 (NCH₂CH₂CH₃), 4.4 (Si(CH₃)₂). IR (DCM): 1662, 1501, 1255, 841, 781. HRMS (ESI⁺, m/z): calculated for C₂₃H₃₇N₂O₃Si ([M+H]⁺) 417.2568. Found 417.2552.

### 9-Hydroxy-6,6-dimethyl-2-propyl-11,11a-dihydro-2H-pyrazino[1,2-b]isoquinoline-1, 4(3H, 6H)-dione

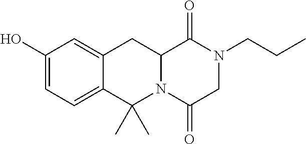 [SMILES: CCCN1CC(=O)N2C(Cc3cc(O)ccc3C2(C)C)C1=O]

HF•pyridine (70%, 0.8 mL) was dissolved in anhydrous THF (20 mL) with anhydrous pyridine (3.18 mL), to give a stock solution of pyridinium fluoride in the concentration of 1.2 M. To 9-((tert-butyldimethylsilyl)oxy)-6,6-dimethyl-2-propyl-2,3,11,11a-tetrahydro-1H-pyrazino[1,2-b]isoquinoline-1,4(6H)-dione (3-xx, 315 mg, 0.76 mmol) in an solution of anhydrous THF (2.5 mL) was added the stock solution (2.5 mL, 3.0 mmol) and stirred for overnight at ambient temperature. The reaction was carefully quenched by the addition of methoxytrimethylsilane (0.62 mL, 3.8 mmol) and stirring for 10 min at ambient temperature. The solution was concentrated under reduced pressure to afford the crude product as a yellow solid. Purified product was obtained by column chromatography on silica gel (66% ethyl acetate in hexane, Rf=0.3) to give pure product as a yellow solid (173 mg, 0.58 mmol, 76% yield). R_{f} (DCM/Et₂O=80/20)=0.15. m.p.=240-241° C. ¹H NMR (400 MHz, CDCl₃) δ: 7.15 (d, J=8.6 Hz, 1H, Ar—H), 6.80 (dd, J=2.5, 8.6 Hz, 1H, Ar—H), 6.76 (d, J=2.5 Hz, 1H, Ar—H), 6.73 (br. s., 1H, OH), 4.07 (dd, J=2.0, 11.9 Hz, 1H, CH₂CH), 4.07 (dd, J=1.5, 17.4 Hz, 1H, COCH₂), 3.97 (dd, J=1.8, 17.4 Hz, 1H, COCH₂), 3.49 (dd, J=2.2, 15.6 Hz, 1H, CH₂CH), 3.56-3.42 (m, 1H, NCH₂CH₂CH₃), 3.40-3.30 (m, 1H, NCH₂CH₂CH₃), 2.99 (dd, J=15.5, 11.7 Hz, 1H, CH₂CH), 1.88 (s, 3H, C(CH₃)₂), 1.86 (s, 3H, C(CH₃)₂), 1.71-1.60 (m, 2H, NCH₂CH₂CH₃), 0.97 (t, J=7.5 Hz, 3H, NCH₂CH₂CH₃). ¹³C NMR (126 MHz, CDCl₃) δ: 164.9 (C═O), 162.1 (C═O), 154.5 (Ar—C), 134.8 (Ar—C), 133.9 (Ar—C), 127.1 (Ar—C), 115.2 (Ar—C), 114.3 (Ar—C), 62.7 (C(CH₃)₂), 56.0 (CH₂CH), 50.4 (COCH₂), 47.7 (NCH₂CH₂CH₃), 36.2 (CH₂CH), 31.8 (C(CH₃)₂), 24.8 (C(CH₃)₂), 19.6 (NCH₂CH₂CH₃), 11.2 (NCH₂CH₂CH₃). IR (Neat): 3276, 1649, 1604, 1314, 863, 756. HRMS (ESI⁺, m/z): calculated for C₁₇H₂₃N₂O₃ ([M+H]⁺) 303.1703. Found 303.1696.

### Fluorination

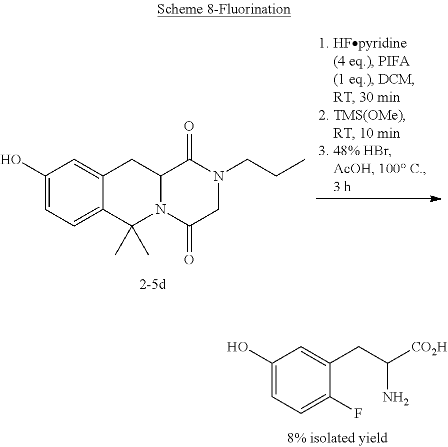 [SMILES: CCCN1CC(=O)N2C(Cc3cc(O)ccc3C2(C)C)C1=O ; NC(Cc1cc(O)ccc1F)C(=O)O]

[Bis(trifluoroacetoxy)iodo]benzene (107 mg, 0.25 mmol) was added to a solution of the tricyclic phenol precursor (76 mg, 0.25 mmol) and HF•pyridine (70%, 26 μL, 1 mmol) in anhydrous DCM (5 mL) at RT. The reaction mixture was stirred for 30 min at RT before being quenched by methoxytrimethylsilane (0.18 mL, 1.3 mmol). The resulting mixture was concentrated under reduced pressure. The residue was then heated in a solution of HBr (48% in H₂O, 1 mL) and acetic acid (1 mL) for 3 h at 100° C. It was then cooled down and neutralised by 20% NaOH in H₂O. The resulting mixture was diluted with 50% MeOH in H₂O, dried azeotropically with acetonitrile and purified by HPLC on a semi-preparative C18 column. Yield=4 mg (8%) as a white solid. ¹H NMR (500 MHz, D₂O) δ: 7.01 (dd, J=9.1, 9.5 Hz, 1H, Ar—H), 6.81-6.76 (m, 1H, Ar—H), 6.76-6.73 (m, 1H, Ar—H), 3.97 (dd, J=5.7, 7.9 Hz, 1H, CHNH₂), 3.26 (dd, J=5.4, 14.5 Hz, 1H, CH₂CH), 3.03 (dd, J=7.9, 14.8 Hz, 1H, CH₂CH). ³C NMR (126 MHz, D₂O) δ: 173.3 (CO₂H), 155.4 (d, J=235.6 Hz, Ar—C—F), 151.7 (d, J=2.9 Hz, Ar—C), 122.8 (d, J=17.2 Hz, Ar—C), 117.7 (d, J=4.8 Hz, Ar—C), 116.4 (d, J=24.8 Hz, Ar—C), 116.0 (d, J=8.6 Hz, Ar—C), 54.9 (CHNH₂), 30.1 (CH₂CH). ¹⁹F NMR (376 MHz, D₂O) δ: −129.0 (dt, J=9.2, 4.6×2 Hz). Characterization data are in agreement with the literature reference.

### Example 8

### Tert-Butyl-meta tyrosine

### Non-Chiral Precursor, Preparation

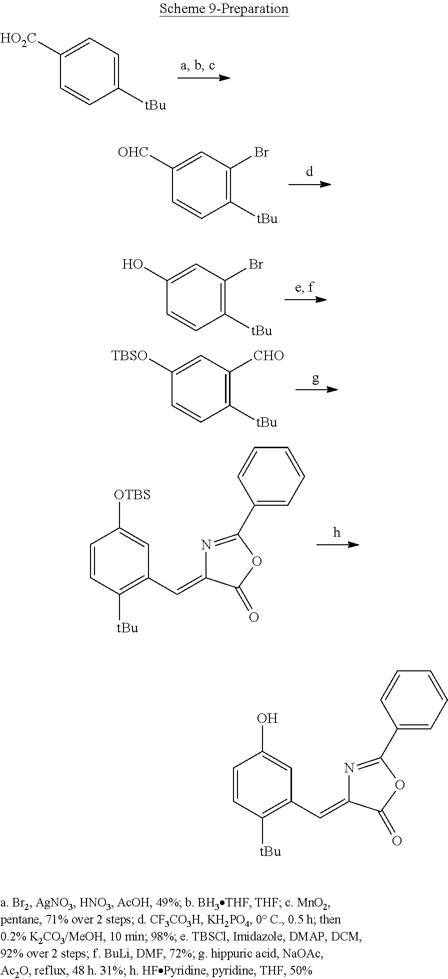

3-Bromo-4-tert-butylbenzoic acid was prepared as literature (Hambley, T. W.; Sternhell, S.; Tansey C. W. Aust. J. Chem. 1990, 43, 807-814.) It's converted to 3-Bromo-4-tert-butylbenzaldehyde with a modified route from the above reference.

### 3-bromo-4-(tert-butyl)benzaldehyde

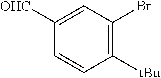 [SMILES: CC(C)(C)c1ccc(C=O)cc1Br]

3-Bromo-4-tert-butylbenzoic acid (18.3 g, 71.2 mmol) was dissolved in anhydrous THF (100 mL) and cooled to 0° C. Borane-THF complex (1.0 M solution in THF, 142 mL, 142 mmol) was added over 1 hour. The reaction mixture was stirred at RT for 12 hours before carefully quenched with 2 M HCl (200 mL). The mixture was extracted with diethyl ether (2×200 mL). The combined organic layer was washed with 2 N HCl (100 mL), water (100 mL) and brine (100 mL), dried, filtered and concentrated to give product as a yellow oil (18.2 g, crude yield 105%). This product was used to the next step without further purification.

The crude 3-bromo-4(tert-butyl)benzaldehyde (maximum 95%, 20 g, 78.4 mmol) was dissolved in petroleum ether (200 mL). Activated manganese dioxide (43.5 g, 500 mmol) was added. The suspension was stirred at RT for 18 h. A 2^{nd} portion of activated manganese dioxide (22 g, 250 mmol) was added. The suspension was stirred for 4 hours. After centrifugation, the supernatant was separated and filtered through a small silica pad (2 g), eluted with petroleum ether (80 mL). The clear filtrate was concentrated to give product as yellow oil (13.4 g, 55.6 mmol) which is NMR pure. Yield over 2 steps 71%. ¹H NMR (200 MHz, CHLOROFORM-d) δ=9.93 (s, 1H), 8.08 (d, J=1.8 Hz, 1H), 7.75 (dd, J=1.8, 8.2 Hz, 1H), 7.61 (d, J=8.2 Hz, 1H), 1.55 (s, 9H). ¹³C NMR (50 MHz, CHLOROFORM-d) δ=190.5, 154.7, 136.8, 135.5, 128.6, 128.1, 123.2, 37.3, 29.4

### 3-bromo-4-(tert-butyl)phenol

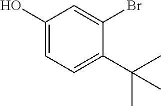

Trifluoroacetic anhydride (20.8 mL, 150 mmol) was slowly added to a suspension of 30% hydrogen peroxide (3.1 mL, 30 mmol) and dichloromethane (40 mL) at 0° C. The mixture was stirred at 0° C. for 1 hour to give the peracid solution. A 1-L flask was charged with potassium phosphate monobasic (54.4 g, 400 mmol), 3-bromo-4-tert-butylbenzaldehyde (4.82 g, 20 mmol) and dichloromethane (200 mL) and cooled to 0° C. The above peracid solution was added over 30 minute with vigorous stirring. The resulting suspension was stirred for another 30 minutes before brine (200 mL) was added followed by sodium sulphite (2.5 g). The mixture was stirred for 5 minutes and diluted with ether (800 mL). The organic layer was washed with water (2×200 mL) and brine (200 mL), dried, filtered and concentrated. The residue was dissolved in methanol (100 mL) containing potassium carbonate (2 mg/mL) and stirred at RT for 10 minutes. The solvent was removed under reduced pressure. The residue was suspended in diethyl ether (200 mL), water (100 mL) and small amount of HCl (2 M, 2 mL). The organic layer was washed with water and brine, dried and concentrated. The crude product was purified by f.c.c (15-20% Et₂O in petroleum ether) to give pale yellow oil (4.49 g, yield 98%). ¹H NMR (400 MHz ,CHLOROFORM-d) δ=7.31 (d, J=8.6 Hz, 1H), 7.13 (d, J=2.8 Hz, 1 H), 6.73 (dd, J=2.8, 8.8 Hz, 1H), 1.49 (s, 9H). ¹³C NMR (101 MHz, CHLOROFORM-d) δ=153.6, 140.1, 128.6, 122.6, 122.4, 113.9, 36.0, 30.0
(3-bromo-4-(tert-butyl)phenoxy) (tert-butyl)dimethylsilane

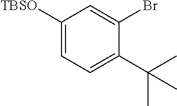 [SMILES: CC(C)(C)c1ccc(O[Si](C)(C)C(C)(C)C)cc1Br]

To a solution of 3-bromo-4-(tert-butyl)phenol (2.29 g, 10.0 mmol), imidazole (1.63 g, 24.0 mmol) and N,N-dimethylaminopyridine (61 mg, 0.5 mmol) in DCM (50 mL) was added tert-butyldimethylsilyl chloride (3.16 g, 21.0 mmol) and the reaction mixture was stirred at ambient temperature for overnight. The reaction was quenched by sat. NaHCO₃ and extracted by DCM (50 mL×2). The combined organic phase was dried in anhydrous MgSO₄, filtered and concentrated in reduced pressure to afford the crude product as light yellow oil. Purified product was obtained by column chromatography on silica gel (pure hexane, Rf=0.7) as colourless oil (2.63 g, 7.7 mmol, 77% yield). ¹H NMR (300 MHz, CDCl₃) ppm 7.06 (d, J=8.6 Hz, 1H, ar-H), 6.90 (d, J=2.6 Hz, 1H, ar-H), 6.50 (dd, J=8.8, 2.6 Hz, 1H, ar-H), 1.27 (s, 9H, CH₃×3 in tBu), 0.78 (s, 9H, CH₃×3 in TBS), 0.00 (s, 6H, CH₃×2 in TBS); ¹³C NMR (75 MHz, CDCl₃) ppm 153.9 (ar-C), 140.4 (ar-C), 128.1 (ar-C), 127.1 (ar-C), 122.4 (ar-C), 118.3 (ar-C), 36.0 (C(CH₃)₃ in tBu), 33.0 (CH₃×3 in tBu), 25.6 (C(CH₃)₃ in TBS), 18.1 (CH₃×3 in TBS), −4.4 (CH₃×2 in TBS);

### 2-(tert-butyl)-5-((tert-butyldimethylsilyl)oxy)benzaldehyde

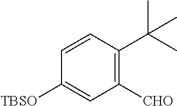 [SMILES: CC(C)(C)c1ccc(O[Si](C)(C)C(C)(C)C)cc1C=O]

n-Butyllithium (2.5 M solution in hexane, 2.1 mL, 5.3 mmol) was added slowly to the solution of (3-bromo-4-(tert-butyl)phenoxy)(tert-butyl)dimethylsilane (1.51 g, 4.4 mmol) in anhydrous THF (50 mL) at −78° C. over 2 min. Anhydrous N,N-dimethylformamide (0.68 mL, 8.8 mmol) was added to the reaction after 30 min at −78° C. and kept for another 30 min before quenching by 1 M HCl (5 mL). The reaction was warmed up to ambient temperature, diluted with diethyl ether (100 mL) and washed by sat. NH₄Cl (50 mL×2). The organic phase was dried in anhydrous MgSO₄, filtered and concentrated in reduced pressure to afford the crude product as light yellow oil. Purified product was obtained by column chromatography on silica gel (2% diethyl ether in hexane, R_{f}=0.2) as colourless oil (924 mg, 3.2 mmol, 72% yield). ¹H NMR (300 MHz, CDCl₃) ppm 10.86 (s, 1H, CHO), 7.45 (d, J=2.7 Hz, 1H, ar-H), 7.38 (d, J=8.8 Hz, 1H, ar-H), 7.00 (dd, J=8.8, 2.7 Hz, 1H, ar-H), 1.54 (s, 9H, CH₃×3 in tBu), 1.04 (s, 9H, CH₃×3 in TBS), 0.27 (s, 6H, CH₃×2 in TBS); ¹³C NMR (75 MHz, CDCl₃) ppm 192.3 (CHO), 153.9 (ar-C), 145.1 (ar-C), 136.4 (ar-C), 127.7 (ar-C), 124.7 (ar-C), 120.6 (ar-C), 35.1 (C(CH₃)³ in tBu), 33.3 (CH₃×3 in tBu), 25.6 (C(CH₃)₃ in TBS), 18.1 (CH₃×3 in TBS), −4.5 (CH₃×2 in TBS); HRMS (ESI, m/z) C₁₇H₂₈NaO₂Si (M+Na⁺) calc. 315.1751. Found 315.1744.

### (Z)-4-(2-(tert-butyl)-5-((tert-butyldimethylsilyl)oxy)benzylidene)-2-phenyloxazol-5(4H)-one

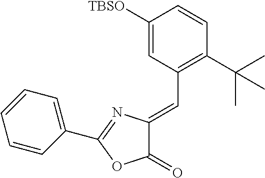 [SMILES: CC(C)(C)c1ccc(O[Si](C)(C)C(C)(C)C)cc1/C=C1\N=C(c2ccccc2)OC1=O]

To a solution of 2-(tert-butyl)-5-((tert-butyldimethylsilyl)oxy)benzaldehyde (802 mg, 2.8 mmol) and hyppuric acid (590 mg, 3.3 mmol) in anhydrous THF (20 mL) was added sodium acetate (54 mg, 0.7 mmol, 24%, mol %) and acetic anhydride (0.66 mL, 6.6 mmol). The reaction was refluxed for 48 hours. The reaction mixture was cooled down, diluted by ethyl acetate and washed by sat. NaHCO₃. The organic phase was dried in anhydrous MgSO₄, filtered and concentrated in reduced pressure to afford the crude product as yellow oil. Purified product was obtained by column chromatography on silica gel (2% diethyl ether in hexane, Rf=0.2) to give pure product as yellow oil (448 mg, 1.0 mmol, 31% yield). ¹H NMR (250 MHz, CDCl₃) ppm 8.19-8.26 (m, 2H, ar-H), 8.16 (d, J=3.0 Hz, 1H, ar-H), 8.06 (s, 1H, C═CH), 7.66 (d, J=7.0 Hz, 1H, ar-H), 7.54-7.62 (m, 2H, ar-H), 7.40 (d, J=8.8 Hz, 1H, ar-H), 6.93 (dd, J=8.8, 2.7 Hz, 1H, ar-H), 1.55 (s, 9H, CH₃×3 in tBu), 1.10 (s, 9H, CH₃×3 in TBS), 0.38 (s, 6H, CH₃×2 in TBS); ¹³C NMR (63 MHz, CDCl₃) ppm 167.9, 163.8, 153.4, 144.3, 133.2, 132.2, 132.1, 132.0, 128.8, 128.2, 127.4, 125.7, 124.7, 122.4, 35.3 (C(CH₃)³ in tBu), 32.4 (CH₃×3 in tBu), 25.7 (C(CH₃)₃ in TBS), 18.2 (CH₃×3 in TBS), −4.3 (CH₃×2 in TBS); HRMS (FI, m/z) C₂₆H₃₃NO₃Si (M⁺) calc. 435.2226. Found 435.2226.

### (Z)-4-(2-(tert-butyl)-5-hydroxybenzylidene)-2-phenyloxazol-5(4H)-one

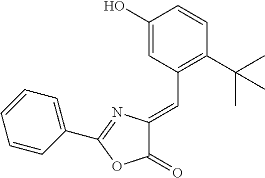 [SMILES: CC(C)(C)c1ccc(O)cc1/C=C1\N=C(c2ccccc2)OC1=O]

Commercial HF•pyridine (70%, 0.8 mL) was dissolved in anhydrous THF (20 mL) with anhydrous pyridine (3.18 mL), to give a stock solution of pyridinium fluoride in the concentration of 1.2 M. To (Z)-4-(2-(tert-butyl)-5-((tert-butyldimethylsilyl)oxy)benzylidene)-2-phenyloxazol-5(4H)-one (338 mg, 0.78 mmol) was added the stock solution (3.25 mL, 3.9 mmol) and stirred for overnight at ambient temperature. The reaction was carefully quenched by the addition of methoxytrimethylsilane (0.65 mL, 4.7 mmol) and stirring for 10 min at ambient temperature. The solution was concentrated under reduced pressure to afford the crude product as yellow oil. Purified product was obtained by column chromatography on silica gel (40% diethyl ether in hexane, Rf=0.4) to give pure product as a yellow solid (126 mg, 0.39 mmol, 50% yield). ¹H NMR (300 MHz, CDCl₃) ppm 8.07-8.12 (m, 2H), 7.94 (d, J=2.9 Hz, 1H), 7.91 (s, 1H), 7.50-7.58 (m, 1H), 7.41-7.48 (m, 2H), 7.29 (d, J=8.6 Hz, 1H), 7.19 (s, 1H), 6.79 (dd, J=8.7, 2.9 Hz, 1H), 1.40 ppm (s, 9H, CH₃×3); ¹³C NMR (75 MHz, CDCl₃) ppm 167.8, 164.1, 153.3, 143.9, 133.4, 132.6, 132.3, 131.8, 128.9, 128.4, 127.

### Non-Chiral Pecursor, Fluorination

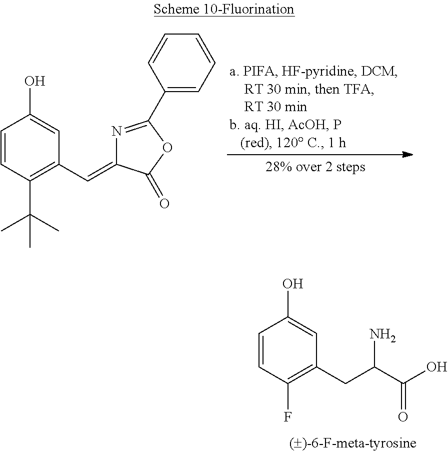 [SMILES: CC(C)(C)c1ccc(O)cc1/C=C1\N=C(c2ccccc2)OC1=O ; NC(Cc1cc(O)ccc1F)C(=O)O]

To a solution of (Z)-4-(2-(tert-butyl)-5-hydroxybenzylidene)-2-phenyloxazol-5(4H)-one (64.3 mg, 0.2 mmol) and HF•pyridine (70% wt, 20.8 □L, 0.8 mmol) in DCM (4 mL) was added phenyliodine di(trifluoroacetace) (PIFA, 86 mg, 0.2 mmol). The reaction was stirred at 25° C. for 30 min. Trifluoroacetic acid (225 □L, 5%) was added and the reaction was stirred for another 30 min before being quenched by methoxytrimethylsilane (0.1 mL). The reaction was concentrated under reduced pressure. Isolated intermediate of oxidative fluorination was obtained by column chromatography on silica gel (50% diethyl ether, Rf=0.5) as pale yellow oil (16 mg, 0.06 mmol, 28% yield). Characterization data are in agreement with the reference compound synthesized independently. The isolated compound was heated with red phosphorus (100 mg) in a solution of HI (˜66%, 1 mL) and acetic acid (1.5 mL) at 120° C. for 1 hour before cooling down and neutralized by 20% aqueous solution of NaOH. The reaction mixture was diluted by water, dried azeotropically with acetonitrile and purified by HPLC (Waters sunfire Prep C18, 10×250 mm, 10 μm, eluted with MeCN/water containing 0.1% TFA at a flow rate of 4 mL/min. The gradient started at 5% MeCN for 3 minutes, then increased to 95% MeCN over 10 minutes, held for 4 minutes, returned to 5% within 2 minutes and equilibrated for 1 minute.). Pure product was obtained as a white solid (11 mg, 0.06 mmol) in 28% yield over two steps. Characterization data are in agreement with the reference compound synthesized independently.

### Chiral Precursor, Preparation

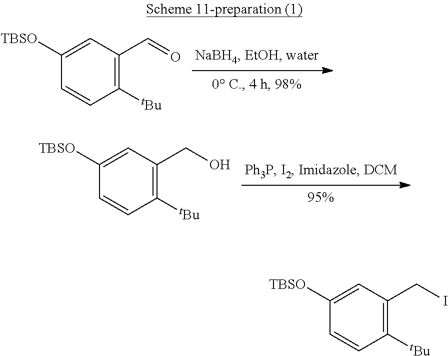 [SMILES: CC(C)(C)c1ccc(O[Si](C)(C)C(C)(C)C)cc1C=O ; CC(C)(C)c1ccc(O[Si](C)(C)C(C)(C)C)cc1CO ; CC(C)(C)c1ccc(O[Si](C)(C)C(C)(C)C)cc1CI]

### (2-(tert-butyl)-5-((tert-butyldimethylsilyl)oxy)phenyl)methanol

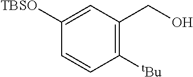 [SMILES: CC(C)(C)c1ccc(O[Si](C)(C)C(C)(C)C)cc1CO]

Sodium borohydride (143 mg, 3.78 mmol) was dissolved in ethanol/water (1:1, 4.4 mL) before use. The aldehyde (2.20 g, 7.53 mmol) was dissolved in ethanol (8.8 mL) and cooled to 0° C. before the above sodium borohydride solution was added over 3 minutes. The reaction mixture was stirred for 5 minutes, then diluted with diethyl ether (100 mL), hexane (30 mL) and water (100 mL). The organic layer was washed with water (2×100 mL) and brine (100 mL), dried and concentrated to give colourless oil (2.12 g, crude yield 95%). This product is used in the next step without further purification. ¹H NMR (400 MHz, CHLOROFORM-d) δ=7.24 (d, J=8.6 Hz, 1H), 7.02 (d, J=2.8 Hz, 1H), 6.70 (dd, J=2.8, 8.6 Hz, 1H), 4.86 (s, 2H), 1.40 (s, 10H), 1.00 (s, 10H), 0.22 (s, 6H). ¹³C NMR (101 MHz, CHLOROFORM-d) δ=153.9, 140.6, 140.3, 127.2, 121.8, 118.4, 63.5, 35.1, 32.1, 25.7, 18.1, −4.4. LRMS (ES−) 293.2 (M−1)

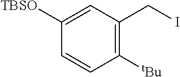 [SMILES: CC(C)(C)c1ccc(O[Si](C)(C)C(C)(C)C)cc1CI]

Triphenylphosphine (2.36 g, 9 mmol) was added to a mixture of iodine (2.34 g, 9.2 mmol) and dichloromethane (44 mL) with a RT water bath. The suspension was stirred for 10 minutes before imidazole (765 mg, 11.25 mmol) was added. The suspension was stirred for another 10 minutes before the above alcohol (2.12 g) was added with dichloromethane (3×3 mL) over 3 minutes. The reaction mixture was stirred for 15 minutes and diluted with hexane (200 mL). The organic suspension was washed with water (100 mL), an aqueous solution of Na₂S₂O₃.5H₂O (0.5 g) in saturated sodium bicarbonate (100 mL) and brine (100 mL), dried and concentrated to give pale yellow solid (4.95 g). This crude product was suspended in hexane (3×20 mL) and quickly filtered through a silica pad (3 g), eluted with hexane (50 mL) and 2% diethyl ether/hexane (50 mL). The collected solution was concentrated to give the iodide as pale yellow oil (2.66 g, 88% over 2 steps). ¹H NMR (400 MHz, CHLOROFORM-d) d=7.14 (d, J=8.6 Hz, 1H), 6.94 (d, J=2.8 Hz, 1H), 6.65 (dd, J=2.8, 8.6 Hz, 1H), 4.74 (s, 2H), 1.43 (s, 9H), 1.00 (s, 9H), 0.22 (s, 6H). ¹³C NMR (101 MHz, CHLOROFORM-d) d=153.8, 140.1, 138.6, 127.5, 125.3, 119.4, 35.2, 31.6, 25.7, 18.2, 7.9, −4.4. LRMS (ES+) 405.2 (M+1)

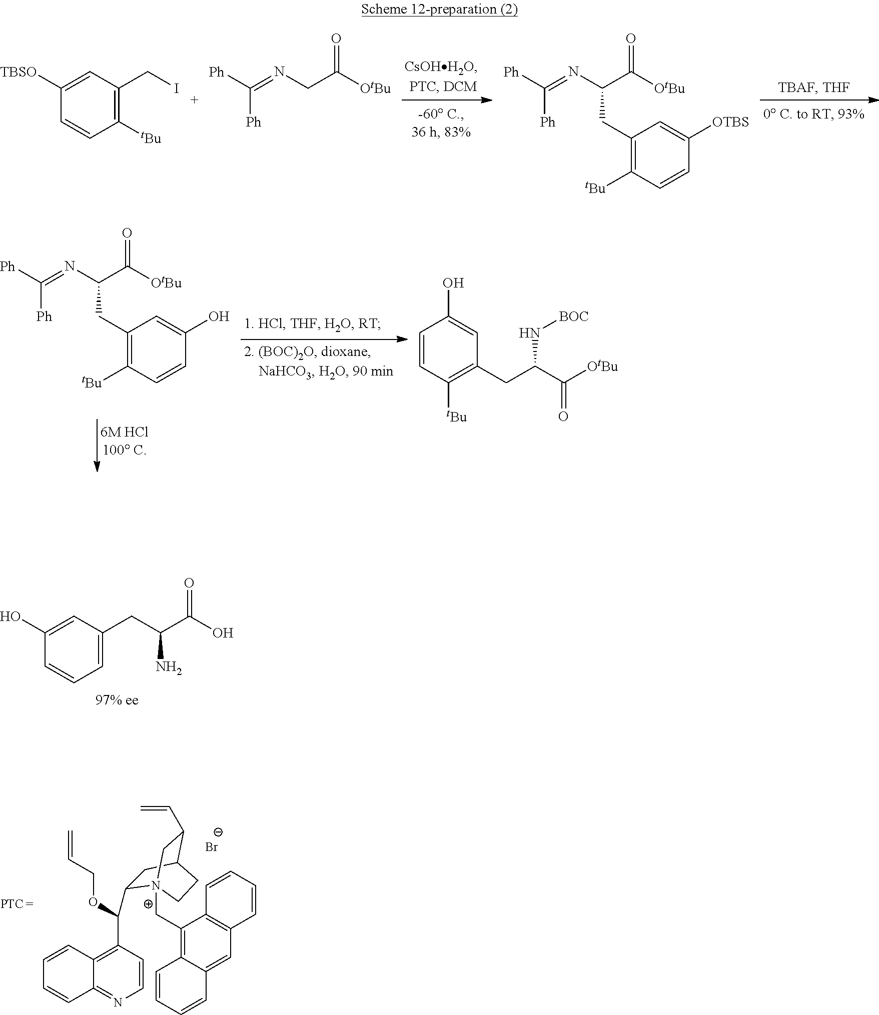

### (S)-tert-butyl 3-(2-(tert-butyl)-5-((tert-butyldimethylsilyl)oxy)phenyl)-2-((diphenylmethylene)amino)propanoate

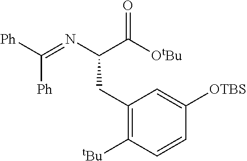 [SMILES: CC(C)(C)OC(=O)[C@H](Cc1cc(O[Si](C)(C)C(C)(C)C)ccc1C(C)(C)C)N=C(c1ccccc1)c1ccccc1]

A solution of tert-butylglycine benzophenone imine (922 mg, 3.12 mmol, 1.2 eq.) and O(9)-allyl-N-(9-anthracenylmethyl)cinchonidinium bromide (315 mg, 0.52 mmol, 0.2 eq.) in dichloromethane (10 mL) was cooled to −60° C. before caesium hydroxide monohydrate (5.24 g, 31.2 mmol, 12 eq.) was added. The iodide (1.05 g, 2.0 mmol) was added with dichloromethane (2+1 mL). The reaction mixture was stirred vigorously at −60° C. for 36 h. The suspension was diluted with ether (50 mL) and water (20 mL), warmed up to RT and further diluted with ether (50 mL) and water (30 mL). The organic layer was washed with water (50 mL) and brine (10 mL), dried and concentrated to give pale yellow oil (1.7 g). This crude product was purified by silica f.c.c eluted with DCM/hexane (50-100%) to give colourless oil (1.62 g) which contains small amount of benzophenone, crude yield 109%. An aliquot was further purified by f.c.c. to give pure product for characterisation. The crude product was subjected to TBS deprotection. HRMS (FI, m/z) C₃₆H₅₀NO₃Si (M⁺) calc. 572.3554. Found 572.3561; ¹H NMR (300 MHz, CHLOROFORM-d) d=7.70-7.61 (m, 2H), 7.41-7.17 (m, 7H), 6.66-6.48 (m, 4H), 4.25 (dd, J=2.8, 10.1 Hz, 1H), 3.69 (dd, J=2.8, 14.0 Hz, 1H), 3.34 (dd, J=10.2, 14.0 Hz, 1H), 1.50 (s, 9H), 1.28 (s, 9H), 0.88 (s, 9H), 0.00 (s, 3H), 0.05 (s, 3H). ¹³C NMR (75 MHz ,CHLOROFORM-d) d=171.2, 170.3, 152.8, 141.0, 139.4, 137.9, 136.5, 130.0, 128.8, 128.0, 127.9, 127.9, 127.7, 126.9, 124.9, 116.8, 81.0, 67.4, 37.6, 35.2, 32.0, 28.1, 25.6, 17.9, −4.5, −4.7.

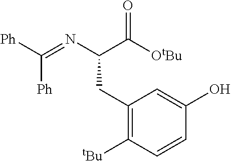 [SMILES: CC(C)(C)OC(=O)[C@H](Cc1cc(O)ccc1C(C)(C)C)N=C(c1ccccc1)c1ccccc1]

The crude TBS ether (1.76 g, ˜92% purity, 2.8 mmol) was dissolved in anhydrous THF (12 mL) and cooled to 0° C. before TBAF (1.0 M in THF, 3.7 mL, 3.7 mmol) was added over 2 minutes. The resulting yellow solution was stirred for 10 minutes at 0° C., then the ice bath was removed and stirring is continued for 15 minutes. Water (50 mL) and ether (100 mL) was added. The aqueous layer was extracted with ether (2×50 mL). The combined organic layer was washed with water (50 mL) and brine (50 mL), dried and concentrated to give pale yellow foam. The product was quickly purified by f.c.c (silica gel, ether/dichloromethane 0-10%) to give 1.20 g of product as yellow solid (crude yield 93%, containing 1% TBSOH by NMR). Further purification on a neutralised column (silica gel +2% NaHCO₃, ether/dichloromethane 0-6%) gave pure product as white foam solid (recovery 58% excluding edge fractions). Total yield over 2 steps was 54%. Mp 52-54° C. [α]_{D}²⁵=−229 (c=1, CHCl₃). ¹H NMR (400 MHz ,CHLOROFORM-d) δ=7.64-7.58 (m, 2H), 7.40-7.21 (m, 6H), 7.18 (d, J=8.8 Hz, 1H), 6.59 (dd, J=3.0, 8.6 Hz, 1H), 6.53 (app. br. d, J=2.8 Hz, 3H), 4.99 (br. s., 1H), 4.25 (dd, J=3.0, 10.1 Hz, 1H), 3.59 (dd, J=3.0, 14.1 Hz, 1H), 3.38 (dd, J=10.2, 14.0 Hz, 1H), 1.48 (s, 9H), 1.26 (s, 9H). ¹³C NMR (101 MHz, CHLOROFORM-d) δ=171.2, 170.5, 152.8, 140.6, 139.3, 137.8, 136.3, 130.1, 128.7, 128.1, 128.0, 127.7, 127.4, 120.0, 112.6, 81.3, 67.4, 37.5, 35.2, 32.0, 28.1

The enantiomeric purity of the product was 97%, determined by chiral HPLC after degradation as below. A small sample (5 mg) was heated with 6 N HCl (0.50 mL) at 100° C. for 10 minutes, cooled to RT and purified by prep HPLC (Waters sunfire Prep C18, 10×250 mm, 10 μm, eluted with MeCN/water containing 0.1% TFA at a flow rate of 4 mL/min. The gradient started at 5% MeCN for 3 minutes, then increased to 95% MeCN over 10 minutes, held for 4 minutes, returned to 5% within 2 minutes and equilibrated for 1 minute.) The product at 4.17 minutes was collected and found to be meta-tyrosine by NMR. It's injected to a CROWNPAK CR(+) column (5 μm, 150×4.0 mm) from Daicel Chemical Industries (Tokyo, Japan) and eluted with aqueous HClO₄ (pH 2.0) at a flow rate of 0.8 mL/min, with racemic meta-tyrosine and commercial L-meta-tyrosine as reference (D-isomer: 6.29 minutes, L-isomer: 8.43 minutes).

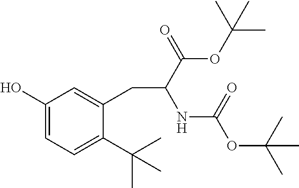 [SMILES: CC(C)(C)OC(=O)NC(Cc1cc(O)ccc1C(C)(C)C)C(=O)OC(C)(C)C]

To a solution of Imine precursor (1.09 mmol, 0.50 g) in THF (10 ml) was added 1.0N HCl (10.9 ml). The resulting solution was stirred under nitrogen for 40 minutes. The reaction mixture was basified by adding solid NaHCO₃ (1.3 g) and extracted three times with EtOAc. The pooled organic layers were washed with brine, dried and evaporated under vacuum to give a white solid. The solid was dissolved in dioxane (6 ml) and water (4 ml), NaHCO3 (2.07 mmol, 0.17 g) and di-tert-butyl dicarbonate (1.20 mmol, 0.26 g) were added. The resulting cloudy mixture was stirred for 1.5 hours after which another portion of di-tert-butyl dicarbonate (0.30 mmol, 0.12 g) was added. Stirring was continued for another 45 minutes after which water was added and the mixture was extracted three times with Et₂O. The pooled organic layers were washed with brine, dried and evaporated under vacuum to give a pale brown oil. The crude product was purified by f.c.c on silica (eluens 10% Et₂O/ hexane to 20% Et₂O/ hexane to 40% Et₂O/ hexane)yield=0.77 g (100%) as a white solid. Rf=0.23 (50% Et₂O/ hexane) m.p 55-58° C. ¹H NMR (400 MHz, METHANOL-d₄) 8 ppm 1.28-1.48 (m, 27H, C(CH₃)₃) 2.94 (dd, J=14.25, 8.9 Hz, 1H, CH₂CH) 3.43 (dd, J=14.25, 6.23 Hz, 1H, CH₂CH) 4.30 (dd, J=8.9, 6.14 Hz, 1H, CH2CH) 6.58 (dd, J=8.62, 2.65 Hz, 1H, Ar—H) 6.70 (d, J=2.73 Hz, 1H, Ar—H) 7.21 (d, J=8.70 Hz, 1H, Ar—H) ¹³C NMR (101 MHz, CHLOROFORM-d) 8 ppm 15.20, 27.88, 28.23, 32.03, 35.12, 35.23, 36.83, 37.98, 54.83, 56.44, 65.85, 76.68, 77.32, 79.95, 80.69, 82.08, 113.19, 113.62, 118.18, 119.77, 127.63, 136.21, 140.02, 153.63, 154.01, 155.31, 172.02. (The NMR was complicated with the rotamers of the carbamate.) IR (neat): 3352, 2975, 1690, 1498, 1366, 1246, 1150 MS (ESI⁻, m/z) 392.26

### Chiral Precursor, Fluorination

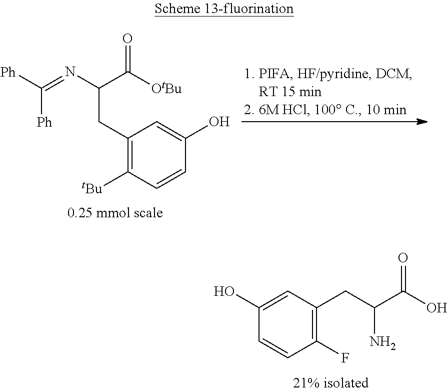 [SMILES: CC(C)(C)OC(=O)C(Cc1cc(O)ccc1C(C)(C)C)N=C(c1ccccc1)c1ccccc1 ; NC(Cc1cc(O)ccc1F)C(=O)O]

HF•pyridine (70%, 26 μL, 1 mmol) was added to a solution of the imine precursor (114 mg, 0.25 mmol) in anhydrous DCM (5 mL) at RT. [Bis(trifluoroacetoxy)iodo]benzene (108 mg, 0.25 mmol) was added 1 minute later. The reaction mixture was stirred for 30 min at RT, quenched by potassium carbonate (276 mg, 2 mmol). The resulting mixture was filtered through cotton wool, rinsed with dichloromethane (3×3 mL). The filtrate was concentrated under reduced pressure. The residue was treated with 6 M HCl (400 μL) at 100° C. fot 10 minutes, cooled down and washed with hexane (2×1 mL). The aqueous layer was filtered though syringe filter and purified by HPLC on a semi-preparative C18 column to give a colourless solid (10.6 mg, yield 21%). The enantiomeric excess was 96%, determined by Chiral HPLC as described above.

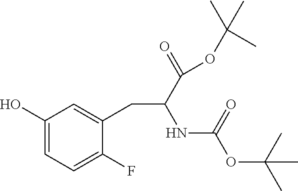 [SMILES: CC(C)(C)OC(=O)NC(Cc1cc(O)ccc1F)C(=O)OC(C)(C)C]

HF•pyridine (70%, 10.1 μL, 0.39 mmol) was added to a solution of N—BOC precursor (0.097 mmol ; 38 mg) in anhydrous DCM (2 mL) at RT. [Bis(trifluoroacetoxy)iodo]benzene (41.7 mg, 0.097 mmol) was added to the reaction mixture 1 min later. The reaction mixture was stirred for 20 min at RT, and then loaded onto a silica column. F.c.c (eluens hexane to 25% Et₂O/hexane to 50% Et₂O/ hexane) yield=8.9 mg as a pale brown oil, that was an inseparable mixture of product and starting material which was purified with prep HPLC (Sunfire Prep C18 column), yield=3.7 mg (10%) of an pale brown oil. Rf=0.17 (50% Et₂O/ hexane), ¹H NMR (300 MHz, CHLOROFORM-d) δ ppm 1.42 (s, 18H, C(CH₃)₃) 2.91-3.12 (m, 2H, CH₂CH) 4.44 (dd, J=14.47, 7.02 Hz, 1H, CH₂CH) 5.11 (d, J=8.04 Hz, 1H, Ar—H) 6.68 (dd, J=7.89, 4.53 Hz, 2H, Ar—H) 6.88 (t, J=8.90 Hz, 1H, Ar—H) ¹⁹F NMR (377 MHz, CHLOROFORM-d) δ ppm −128.53, −128.15

### ¹⁸F Fluorination

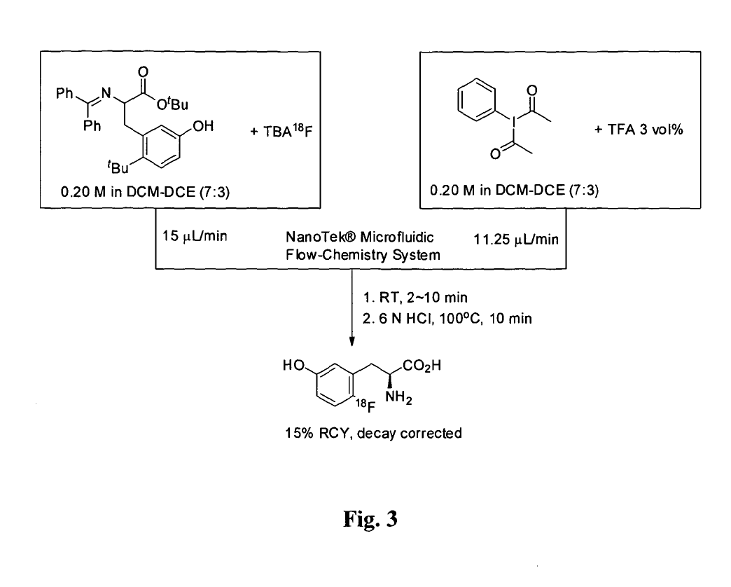

FIG. 3 shows schematically a method of radiolabelling a chiral precursor to 6-¹⁸F-meta-tyrosine. with a microfluidic apparatus (NanoTek®, Advion).

General Radiolabelling with Microfluidic Apparatus (NanoTek®, Advion)

The radiosynthesis and azeotropic drying was automatically performed on a commercial microreactor device (NanoTek®, Advion). Cyclotron-produced non-carrier-added aqueous [¹⁸F]fluoride was first adsorbed onto an anion-exchange cartridge and subsequently released with 500 μl solution of tBu₄NHCO₃ (25 mg) in acetonitrile/water (4:1) into the concentrator. The solution was dried with two cycles of azeotropic drying with acetonitrile (300 μl) and dissolved in CH₂Cl₂/ClCH₂CH₂Cl (7:3 v/v, 500 μl) containing the substrate (0.1-0.5 M). In a separate solution, the oxidant (0.1-0.5 M) was dissolved in CH₂Cl₂/ClCH₂CH₂Cl (7:3 v/v) containing trifluoroacetic acid (3%). Both solutions were delivered at various flow rates (8-30 l/min) through the microfluidic reactor at ambient temperature or higher temperature if required. The reaction mixture was treated under corresponding work up conditions to get the final product.

Chemical identity was verified with radio-HPLC using the Gilson 322 system, equipped with a NaI/PMT radiodetector and a UV-detector using the analytical Waters Nova-Pak C18 Column (4 μm, 3.9×150 mm). The enantiomeric purity of the product was determined after HPLC purification using CROWNPAK CR(+) column (5 μm, 150×4.0 mm) from Daicel Chemical Industries (Tokyo, Japan) and eluted with aqueous HClO₄ (pH 2.0) at a flow rate of 0.8 mL/min. The retention time of two enantiomers was 8.56 min (L-) and 6.53 min (D-), identified by coinjection with the racemic or enantioenriched reference compounds.

### A Typical Discovery Procedure is Show Below.

The precursor solution (0.20 M in CH₂Cl₂/ClCH₂CH₂Cl, 7:3 v/v, 500 μl) was mixed well with dry [¹⁸F]-TBAF and filled the reagent loop. Another reagent loop was filled with the oxidant (PIDA, 0.20 M in CH₂Cl₂/ClCH₂CH₂Cl, 7:3 v/v, 1000 μl, with 30 μl of TFA). The reaction was carried out at 25° C. with a precursor flow rate of 15 μl/min and oxidant/precursor flow ratio of 0.75. 30 μl of precursor was delivered to give the fluorination solution (32.40 MBq at 0 minutes). The solvent was dried under nitrogen flow and the resulting mixture was treated with 6N HCl at 100° C. for 10 minutes and cooled to RT to give the crude product (9.512 MBq at 22 minutes). An aliquot (0.35 MBq at 47 minutes) was injected to HPLC and [¹⁸F]6-fluoro-meta-tyrosine was collected (0.152 MBq at 56 minutes). RCY 15% (decay corrected).

### Example 9

### Catechol

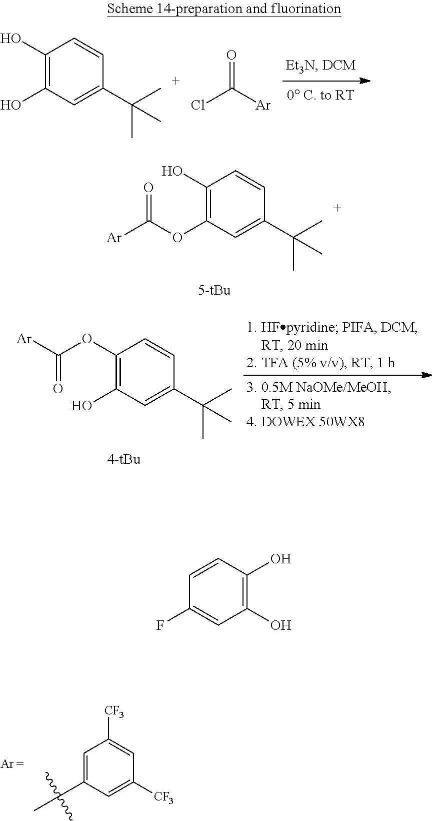

### 5- and 4-tert-Butyl-2-hydroxyphenyl 3,5-bis(trifluoromethyl)benzoate

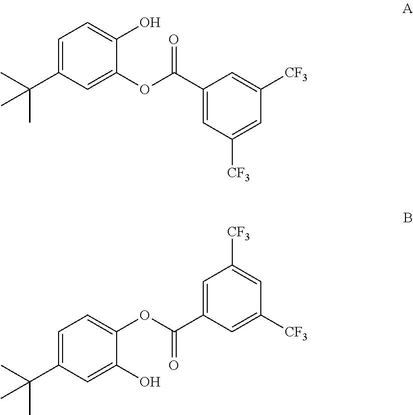 [SMILES: CC(C)(C)c1ccc(O)c(OC(=O)c2cc(C(F)(F)F)cc(C(F)(F)F)c2)c1 ; CC(C)(C)c1ccc(OC(=O)c2cc(C(F)(F)F)cc(C(F)(F)F)c2)c(O)c1]

3,5-Bis(trifluoromethyl)benzoyl chloride (901 μL, 5 mmol) was dissolved in DCM (5 mL) was added to a solution of 4-tert-butylcatechol (831 mg, 5 mmol) and Et₃N (1.39 mL, 10 mmol) in DCM (10 mL) at 0° C. The reaction mixture was stirred for 1 h at RT. The reaction mixture was then quenched with H₂O. The aqueous layer was extracted with DCM. The combined organic extracts were dried over anhydrous Na₂SO₄ and concentrated under reduced pressure. The crude product was purified by f.c.c. (eluent: DCM/pet. ether=50/50 to 100/0), yield=1.36 g (67%) as white solid. m.p.=46-50° C. R_{f} (DCM/pet. ether=1/1): 0.13. Ratio of A:B=2:3. ¹H NMR (500 MHz, CDCl₃) δ: 8.67 (d, J=8.2 Hz, 2H, Bz-H), 8.16 (s, 1H, Bz-H), 7.22 (dd, J=2.4, 8.4 Hz, 0.4H_{A}, Ph-H), 7.18 (d, J=2.5 Hz, 0.4H_{A}, PhH), 7.12-7.09 (d, J=8.5 Hz, 0.6H_{B}, Ph-H), 7.06 (d, J=2.2 Hz, 0.6H_{B}, Ph-H), 7.02 (dd, J=2.2, 8.5 Hz, 0.6H_{B}, Ph-H), 6.95 (d, J=8.5 Hz, 0.4H_{A}, Ph-H), 5.33 (br. s., 1H, OH), 1.32 (s, 9H, C(CH₃). ¹³C NMR (126 MHz, CDCl₃) δ: 162.6 (C═O), 162.5 (C═O), 151.4, 146.1, 145.1, 144.3, 137.9, 135.9, 132.5, (q, J=34 Hz), 131.3, 131.3, 130.4 (septet), 127.1 (septet), 124.6, 122.7 (q, J=273 Hz), 121.7, 119.4, 118.5, 117.7, 115.4, 34.6 (C(CH₃)₃), 34.3 (C(CH₃)₃), 31.4 (C(CH₃)₃), 31.3 (C(CH₃)₃). ¹⁹F NMR (377 MHz, CDCl₃) δ: −62.9. IR (DCM): 3424, 2968, 1754, 1730, 1280, 1242, 1184, 1141.

HRMS (FI⁺, m/z): calculated for C₁₇H₁₆F₆O₃ (M⁺) 406.1004. Found 406.1006. HF•pyridine (70%, 26 μL, 1 mmol) was added to the catechol mono-ester (0.25 mmol) in anhydrous DCM (5 mL). [Bis(trifluoroacetoxy)iodo]benzene (107 mg, 0.25 mmol) was added to the reaction mixture 1 min later. The reaction mixture was stirred for 20 min at RT. Trifluoroacetic acid (250 μL, 3.3 mmol, 5% v/v) was then added and stirred for 1 h at RT. Solvent and trifluoroacetic acid were removed under reduced pressure. The residue was treated with a solution of sodium methoxide in MeOH (0.5 M, 4 mL) under argon at RT for 5 min. DOWEX 50WX8 (2.5 mL) was added and stirred for 5 min at RT. The mixture was filtered and the resin was washed with MeOH (1 mL). The filtrate was concentrated under reduced pressure, dissolved in DMSO-d₆ and subjected to ¹⁹F NMR. Yield (14-18%) of the product was calculated based on ¹⁹F NMR peak intergration at δ-124.2 ppm (in) with the addition of 2,4-dibromofluorobenzene in DMSO-d₆ (0.25 M, 100 μL, 0.25 mmol, δ-109.9 ppm) to the NMR sample as the internal reference. The identity of the product was confirmed by ¹⁹F NMR with addition of approximately equal amount of 4-fluorocatechol (0.25 M) to the NMR sample.

1. A process for producing an ¹⁸F-labelled compound, the process comprising:
treating a compound of formula (I) 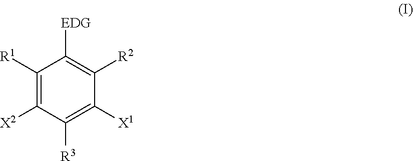
wherein
EDG is an electron-donating group selected from —OH, —OR⁴, —NHR⁵ and —N(R⁵⁵)(R⁵);
R⁴ is unsubstituted or substituted C₁₋₂₀ alkyl, unsubstituted or substituted acyl, unsubstituted or substituted aryl, unsubstituted or substituted C₃₋₁₀ cycloalkyl, or —SiR⁶⁶R⁶R⁷; wherein R⁶⁶, R⁶ and R⁷, which are the same or different, are independently selected from H, unsubstituted or substituted C₁₋₂₀ alkyl, unsubstituted or substituted aryl, and unsubstituted or substituted C₃₋₁₀ cycloalkyl, and unsubstituted or substituted C₁₋₂₀ alkoxy;
R⁵ is selected from —C(O)OR⁸, —S(O)₂R⁹, unsubstituted or substituted C₁₋₂₀ alkyl, unsubstituted or substituted aryl, unsubstituted or substituted C₃₋₁₀ cycloalkyl, acyl, and —SiR⁶⁶R⁶R⁷, provided that R⁵ and R¹ or R⁵ and R² may together form a bidentate group L², wherein L² is -alk-, —C(O)-alk-, —C(O)O-alk- or —S(O)₂-alk- wherein -alk- is unsubstituted or substituted C₁₋₃ alkylene; wherein R⁶⁶, R⁶ and R⁷, which are the same or different, are independently selected from H, unsubstituted or substituted C₁₋₂₀ alkyl, unsubstituted or substituted aryl, unsubstituted or substituted C₃₋₁₀ cycloalkyl, and unsubstituted or substituted C₁₋₂₀ alkoxy; wherein R⁸ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R⁹ is unsubstituted or substituted aryl or unsubstituted or substituted C₁₋₂₀ alkyl;
R⁵⁵ is selected from —C(O)OR⁸, —S(O)₂R⁹, unsubstituted or substituted C₁₋₂₀ alkyl, unsubstituted or substituted aryl, unsubstituted or substituted C₃₋₁₀ cycloalkyl, acyl, and —SiR⁶⁶R⁶R⁷; wherein R⁶⁶, R⁶ and R⁷, which are the same or different, are independently selected from H, unsubstituted or substituted C₁₋₂₀ alkyl, unsubstituted or substituted aryl, unsubstituted or substituted C₃₋₁₀ cycloalkyl, and unsubstituted or substituted C₁₋₂₀ alkoxy; wherein R⁸ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R⁹ is unsubstituted or substituted aryl or unsubstituted or substituted C₁₋₂₀ alkyl;
R¹ and R², which are the same or different, are independently selected from H, unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, acyl, amido, acylamido, halo, cyano, —OR¹⁰ and —NR¹¹R¹¹,
provided that when EDG is —NHR⁵ or —N(R⁵⁵)(R⁵), R⁵ and R¹ or R⁵ and R² may together form a bidentate group L² wherein L² is -alk-, —C(O)-alk-, —C(O)O-alk- or —S(O)₂-alk- wherein -alk- is unsubstituted or substituted C₁₋₃ alkylene,
and provided that R¹ and X² may together form a bidentate group such that R¹, X² and the ring carbon atoms to which R¹ and X² are bonded together form an unsubstituted or substituted fused aryl, heteroaryl, C₅₋₈ carbocyclic or C₅₋₈ heterocyclic ring;
and provided that R² and X¹ may together form a bidentate group such that R², X¹ and the ring carbon atoms to which R² and X¹ are bonded together form an unsubstituted or substituted fused aryl, heteroaryl, C₅₋₈ carbocyclic or C₅₋₈ heterocyclic ring;
R¹⁰ is a hydroxyl protecting group;
R¹¹ and R¹¹¹, which are the same or different, are independently selected from unsubstituted or substituted C₁₋₂₀ alkyl, unsubstituted or substituted C₃₋₁₀ cycloalkyl, C₁₋₂₀ perfluoroalkyl, acyl, unsubstituted or substituted aryl, perfluoroaryl, unsubstituted or substituted heteroaryl, unsubstituted or substituted C₃₋₁₀ heterocyclyl, —C(O)OR¹⁶ and —S(O)₂R¹⁷, wherein R¹⁶ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R¹⁷ is unsubstituted or substituted aryl or unsubstituted or substituted C₁₋₁₀ alkyl;
X¹ and X², which are the same or different, are independently selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, unsubstituted or substituted C₁₋₂₀ alkoxy, amino, unsubstituted or substituted C₁₋₁₀ alkylamino, unsubstituted or substituted di(C₁₋₁₀)alkylamino, unsubstituted or substituted acyl, unsubstituted or substituted amido, unsubstituted or substituted acylamido, halo, cyano and a group of formula (X), formula (X2), formula (Y), formula (Z1) or formula (Z2) 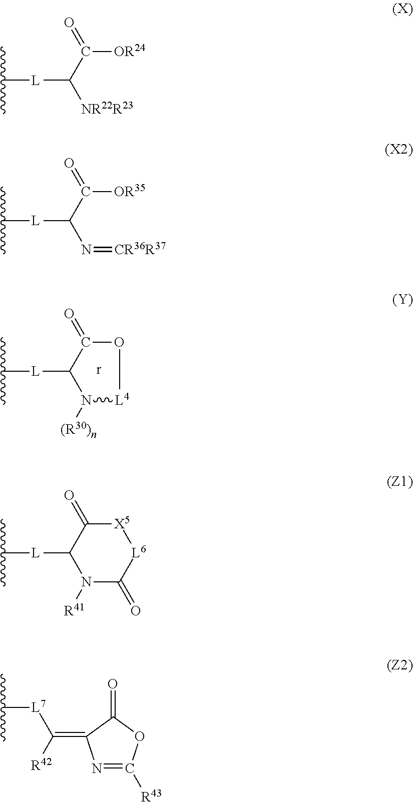
wherein
L⁵ is unsubstituted or substituted C₁₋₆ alkylene;
R⁴⁰ is an amino protecting group;
L is unsubstituted or substituted C₁₋₄ alkylene;
R²² and R²³, which are the same or different, are independently selected from H and an amino protecting group;
R²⁴ is H or a carboxyl protecting group;
R³⁵ is H or a carboxyl protecting group;
R³⁶ and R³⁷, which are the same or different, are independently selected from unsubstituted or substituted aryl, unsubstituted or substituted heteroaryl, unsubstituted or substituted C₃₋₁₀ heterocyclyl, unsubstituted or substituted C₁₋₂₀ alkyl, or unsubstituted or substituted C₃₋₁₀ cycloalkyl, provided that R³⁶ and R³⁷ may together form an unsubstituted or substituted C₄₋₆ alkylene alkylene group;
R³⁰ is H, unsubstituted or substituted C₁₋₁₀ alkyl, or unsubstituted or substituted aryl;
n is 0 or 1, provided that when n is 0, the bond between L⁴ and N is a double bond and when n is 1, the bond between L⁴ and N is a single bond;
L⁴ is a linking group wherein L⁴ forms, together with the —N(R³⁰)ₙ—C(L)-C(O)—O— moiety to which L⁴ is bonded, a ring r which is a C₅₋₈ heterocyclic ring or a C₅₋₈ heteroaryl ring;
R⁴¹ is H or an amino protecting group, provided that when R³ is X⁴, R⁴¹ may be a single bond which connects X⁴ to said group of formula (Z1);
X⁵ is NR⁴⁴ or O, wherein R⁴⁴ is selected from unsubstituted or substituted C₁₋₁₀ alkyl, unsubstituted or substituted C₃₋₁₀ cycloalkyl, unsubstituted or substituted aryl, unsubstituted or substituted heteroaryl, and unsubstituted or substituted C₃₋₁₀ heterocyclyl;
L⁶ is substituted or unsubstituted C₁₋₃ alkylene;
L⁷ is a bond or an unsubstituted or substituted C₁₋₄ alkylene group;
R⁴² is H, unsubstituted or substituted C₁₋₁₀ alkyl, or unsubstituted or substituted aryl;
R⁴³ is unsubstituted or substituted aryl, unsubstituted or substituted heteroaryl, unsubstituted or substituted C₃₋₁₀ heterocyclyl, unsubstituted or substituted C₁₋₂₀ alkyl, or unsubstituted or substituted C₃₋₁₀ cycloalkyl;
provided that X² and R¹ may together form a bidentate group such that R¹, X² and the ring carbon atoms to which R¹ and X² are bonded together form an unsubstituted or substituted fused aryl, heteroaryl, C₅₋₈ carbocyclic or C₅₋₈ heterocyclic ring;
and provided that X¹ and R² may together form a bidentate group such that R², X¹ and the ring carbon atoms to which R² and X¹ are bonded together form an unsubstituted or substituted fused aryl, heteroaryl, C₅₋₈ carbocyclic or C₅₋₈ heterocyclic ring;
and provided that when X¹ or X² is substituted C₁₋₂₀ alkyl, substituted -L⁵-N(R⁴⁰)H, substituted C₃₋₂₀ cycloalkyl, substituted aryl, substituted heteroaryl, substituted C₃₋₁₀ heterocyclyl, substituted C₁₋₂₀ alkoxy, substituted C₁₋₁₀ alkylamino, substituted di(C₁₋₁₀)alkylamino, substituted acyl, substituted amido, substituted acylamido, or a group of formula (X), formula (X2), formula (Y), formula (Z1) or formula (Z2), said X¹ or X² may be substituted with a group X⁴, wherein X⁴ is a bidentate cleavable surrogate group which is bonded (a) to said X¹ or X² and (b) to the ring carbon atom para to EDG;
R³ is selected from H, X³ and X⁴, wherein X³ is a monodentate cleavable surrogate group and X⁴ is said bidentate cleavable surrogate group;
with [¹⁸F]fluoride in the presence of an oxidant,
thereby producing, when R³ in the compound of formula (I) is H, an ¹⁸F-labelled compound of formula (II): 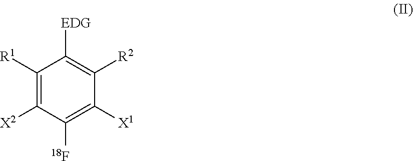
wherein EDG, R¹, R², X¹ and X² are as defined above,
or thereby producing, when R³ in the compound of formula (I) is said monodentate cleavable surrogate group X³, a compound of formula (IIa): 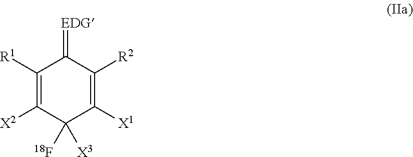
wherein EDG′ is O, NR⁵, [OR⁴]⁺ or [NR⁵⁵R⁵]⁺ and wherein R⁴, R⁵, R⁵⁵, R¹, R², X¹, X² and X³ are as defined above,
or thereby producing, when R³ in the compound of formula (I) is said bidentate cleavable surrogate group X⁴, a compound of formula (Ic) or a compound of formula (IId): 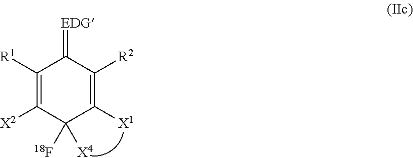
wherein EDG′ is O, NR⁵, [OR⁴]⁺ or [NR⁵⁵R⁵]⁺ and wherein R⁴, R⁵, R⁵⁵, R¹, R² and X² are as defined above; and wherein X¹ is a C₁₋₂₀ alkyl, -L⁵-N(R⁴⁰)H, C₃₋₂₀ cycloalkyl, aryl, heteroaryl, C₃₋₁₀ heterocyclyl, C₁₋₂₀ alkoxy, C₁₋₁₀ alkylamino, di(C₁₋₁₀)alkylamino, acyl, amido or acylamido group, or a group of formula (X), formula (X2), formula (Y), formula (Z1) or formula (Z2), provided that X¹ is substituted with X⁴, wherein X⁴ is said bidentate cleavable surrogate group which is bonded (a) to X¹ and (b) to the ring carbon atom para to EDG′; 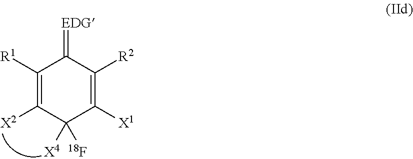
wherein EDG′ is O, NR⁵, [OR⁴]⁺ or [NR⁵⁵R⁵]⁺ and wherein R⁴, R⁵, R⁵⁵, R¹, R² and X¹ are as defined above; and wherein X² is a C₁₋₂₀ alkyl, -L⁵-N(R⁴⁰)H, C₃₋₂₀ cycloalkyl, aryl, heteroaryl, C₃₋₁₀ heterocyclyl, C₁₋₂₀ alkoxy, C₁₋₁₀ alkylamino, di(C₁₋₁₀)alkylamino, acyl, amido or acylamido group, or a group of formula (X), formula (X2), formula (Y), formula (Z1) or formula (Z2), provided that X² is substituted with X⁴, wherein X⁴ is said bidentate cleavable surrogate group which is bonded (a) to X² and (b) to the ring carbon atom para to EDG′.

2. A process according to claim 1, for producing an ¹⁸F-labelled compound, the process comprising treating a compound of formula (I) 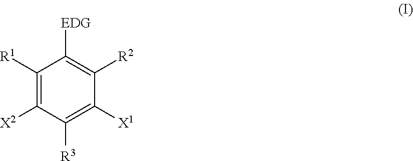
wherein
EDG is an electron-donating group selected from —OH, —OR⁴ and —NHR⁵;
R⁴ is unsubstituted or substituted C₁₋₂₀ alkyl, unsubstituted or substituted aryl, unsubstituted or substituted C₃₋₁₀ cycloalkyl, or —SiR⁶⁶R⁶R⁷; wherein R⁶⁶, R⁶ and R⁷, which are the same or different, are independently selected from H, unsubstituted or substituted C₁₋₂₀ alkyl, unsubstituted or substituted aryl, and unsubstituted or substituted C₃₋₁₀ cycloalkyl, and unsubstituted or substituted C₁₋₂₀ alkoxy;
R⁵ is selected from —C(O)OR⁸, —S(O)₂R⁹, unsubstituted or substituted C₁₋₂₀o alkyl, unsubstituted or substituted aryl, unsubstituted or substituted C₃₋₁₀ cycloalkyl, acyl, and —SiR⁶⁶R⁶R⁷, provided that R⁵ and R¹ or R⁵ and R² may together form a bidentate group L², wherein L² is -alk-, —C(O)-alk-, —C(O)O-alk- or —S(O)₂-alk- wherein -alk- is unsubstituted or substituted C₁₋₃ alkylene; wherein R⁶⁶, R⁶ and R⁷, which are the same or different, are independently selected from H, unsubstituted or substituted C₁₋₂₀ alkyl, unsubstituted or substituted aryl, unsubstituted or substituted C₃₋₁₀ cycloalkyl, and unsubstituted or substituted C₁₋₂₀ alkoxy; wherein R⁸ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R⁹ is unsubstituted or substituted aryl or unsubstituted or substituted C₁₋₂₀ alkyl;
R¹ and R², which are the same or different, are independently selected from H, unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, acyl, amido, acylamido, halo, cyano, —OR¹⁰ and —NR¹¹R¹¹¹, provided that when EDG is NR⁵, R⁵ and R¹ or R⁵ and R² may together form a bidentate group L², wherein L² is -alk-, —C(O)-alk-, —C(O)O-alk- or —S(O)₂-alk- wherein -alk- is unsubstituted or substituted C₁₋₃ alkylene;
R¹⁰ is a hydroxyl protecting group;
R¹¹ and R¹¹¹, which are the same or different, are independently selected from unsubstituted or substituted C₁₋₂₀ alkyl, unsubstituted or substituted C₃₋₁₀ cycloalkyl, C₁₋₂₀ perfluoroalkyl, acyl, unsubstituted or substituted aryl, perfluoroaryl, unsubstituted or substituted heteroaryl, unsubstituted or substituted C₃₋₁₀ heterocyclyl, —C(O)OR¹⁶ and —S(O)₂R¹⁷, wherein R¹⁶ is selected from unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, and 9-fluorenylmethyl; and wherein R¹⁷ is unsubstituted or substituted aryl or unsubstituted or substituted C₁₋₁₀ alkyl;
R³ is selected from H and X³, wherein X³ is a cleavable surrogate group;
X¹ and X², which are the same or different, are independently selected from H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X) 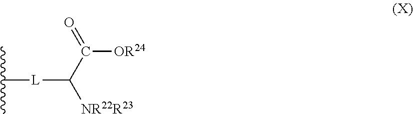
wherein
L is unsubstituted or substituted C₁₋₄ alkylene;
R²² and R²³, which are the same or different, are independently selected from H and an amino protecting group; and
R²⁴ is H or a carboxyl protecting group;
with [¹⁸F]fluoride in the presence of an oxidant,
thereby producing, when R³ in the compound of formula (I) is H, an ¹⁸F-labelled compound of formula (II): 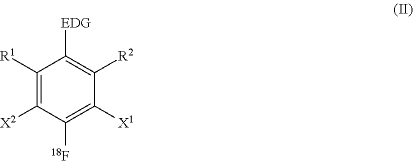
wherein EDG, R¹, R², X¹ and X² are as defined above,
or, when R³ in the compound of formula (I) is said cleavable surrogate group X³, thereby producing a compound of formula (IIa): 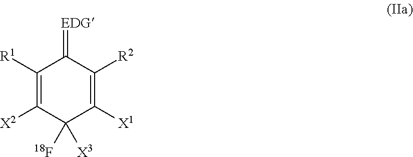
wherein EDG′ is O, NR⁵ or [OR⁴]⁺, and wherein R⁴, R⁵, R¹, R², X¹, X² and X³ are as defined above.

3. A process according to claim 1, wherein R³ in the compound of formula (I) is said cleavable surrogate group X³ and the process further comprises rearomatisation of the compound of formula (IIa) to produce a compound of formula (II) 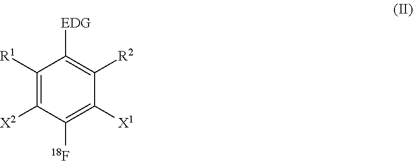
wherein EDG, R¹, R², X¹ and X² are as defined in claim 1.

4. A process according to claim 3 wherein said rearomatisation is performed in situ.

5. A process according to claim 3 wherein said rearomatisation comprises the addition of a reagent which effects cleavage of X³ from the carbon atom of the ring which is para to EDG′ in the compound of formula (IIa), to produce a compound of formula (II), wherein said reagent is an acid, base or oxidising agent.

6.-13. (canceled)

14. A process according to claim 1 wherein R³ in the compound of formula (I) is said cleavable surrogate group X³, and wherein one of X¹ and X² in the compound of formula (I) is a group of formula (Z2): 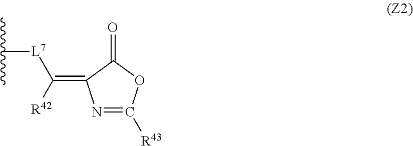
wherein L⁷, R⁴² and R⁴³ are as defined in claim 1;
wherein the process further comprises
(i) rearomatisation of the compound of formula (IIa), comprising cleavage of X³ from the ring carbon atom para to EDG′ in said compound; and
(ii) performing a reductive hydrolysis, in order to convert said group of formula (Z2) into a group of formula (Z3): 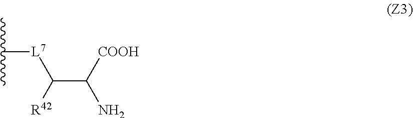
wherein L⁷ and R⁴² are as defined in claim 1 for the group of formula (Z2);
thereby producing a compound of formula (IIZ) 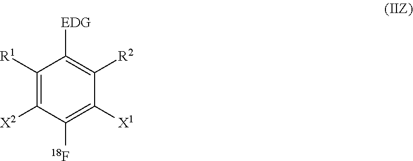
wherein EDG, R¹ and R² are as defined in claim 1, one of X¹ and X² is a said group of formula (Z3), and the other of X¹ and X² is as defined in claim 1.

15.-17. (canceled)

18. A process according to claim 14 wherein EDG is OH, R¹ and R² are both H, L⁷ is a single bond, R⁴² is H, the other of X¹ and X² is H, and the compound of formula (IIZ) is as follows:

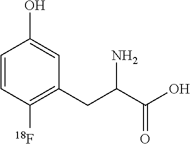 [SMILES: NC(Cc1cc(O)ccc1[18F])C(=O)O]

19. A process according to claim 1, wherein R³ in the compound of formula (I) is said cleavable surrogate group X³, and wherein one of X¹ and X² in the compound of formula (I) is a group of formula (X2) 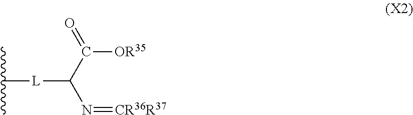
wherein R³⁵, R³⁶ and R³⁷ are as defined in claim 1;
wherein the process further comprises
(i) rearomatisation of the compound of formula (IIa), comprising cleavage of X³ from the ring carbon atom para to EDG′ in said compound; and
(ii) a deprotection step, comprising converting said N═CR³⁶R³⁷ group in the group of formula (X2) into NH₂ and, when R³⁵ is a carboxyl protecting group, substituting H for said carboxyl protecting group, thereby converting the group of formula (X2) into a group of formula (X³): 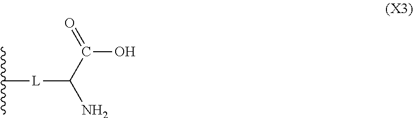
wherein L is as defined in claim 1;
thereby producing a compound of formula (IIX) 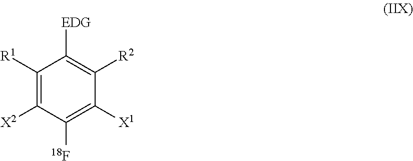
wherein EDG, R¹ and R² are as defined in claim 1, one of X¹ and X² is a said group of formula (X³), and the other of X¹ and X² is as defined in claim 1.

20.-21. (canceled)

22. A process according to claim 19 wherein EDG is OH, R¹ and R² are both H, L is CH₂, the other of X¹ and X² is H, and the compound of formula (IIX) comprises:

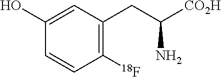 [SMILES: N[C@@H](Cc1cc(O)ccc1[18F])C(=O)O]

23. A process according to claim 1 wherein R³ in the compound of formula (I) is said bidentate cleavable surrogate group X⁴ and the process further comprises rearomatisation of the compound of formula (IIc) or (IId) to produce a compound of formula (IIc′) or (IId′) respectively: 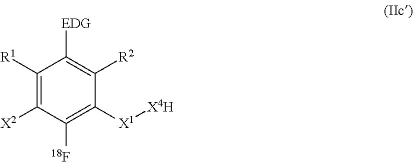
wherein EDG, R¹ and R² are as defined in claim 1;
wherein X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2) as defined in claim 1;
wherein X¹ is a C₁₋₂₀ alkyl, -L⁵-N(R⁴⁰)H, C₃₋₂₀ cycloalkyl, aryl, heteroaryl, C₃₋₁₀ heterocyclyl, C₁₋₂₀ alkoxy, C₁₋₁₀ alkylamino, di(C₁₋₁₀)alkylamino, acyl, amido or acylamido group, or a group of formula (X), formula (X2), formula (Y), formula (Z1) or formula (Z2), wherein X¹ is substituted with X⁴; and
wherein X⁴ is said bidentate cleavable surrogate group which is bonded (a) to X¹ and (b) to H; 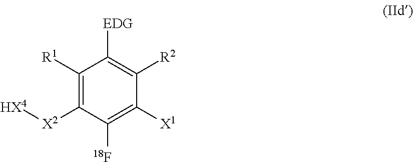
wherein EDG, R¹ and R² are as defined in claim 1;
wherein X¹ is selected from H, unsubstituted or substituted -L⁵-N(R⁴⁰)H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2), as defined in claim 1;
wherein X² is a C₁₋₂₀ alkyl, -L⁵-N(R⁴⁰)H, C₃₋₂₀ cycloalkyl, aryl, heteroaryl, C₃₋₁₀ heterocyclyl, C₁₋₂₀ alkoxy, C₁₋₁₀ alkylamino, di(C₁₋₁₀)alkylamino, acyl, amido or acylamido group, or a group of formula (X), formula (X2), formula (Y), formula (Z1) or formula (Z2), wherein X² is substituted with X⁴; and
wherein X⁴ is said bidentate cleavable surrogate group which is bonded (a) to X² and (b) to H.

24. A process according to claim 1 wherein R³ in the compound of formula (I) is said bidentate cleavable surrogate group, X⁴, and wherein either:
(a) X¹ is a said group of formula -L⁵-N(R⁴⁰)H which is substituted with said bidentate cleavable surrogate group, X⁴, to form a group of formula *-L⁵-N(R⁴⁰)—X⁴-**, wherein * is the point of attachment of X¹ to the ring carbon atom meta to EDG or EDG′ and ** is the point of attachment of X⁴ to the ring carbon atom para to EDG or EDG′; or
(b) X² is a said group of formula -L⁵-N(R⁴⁰)H which is substituted with said bidentate cleavable surrogate group, X⁴, to form a group of formula *-LS-N(R⁴⁰)—X⁴-**, wherein * is the point of attachment of X² to the ring carbon atom meta to EDG or EDG′ and ** is the point of attachment of X⁴ to the ring carbon atom para to EDG or EDG′;
and the process further comprises rearomatisation of the compound of formula (IIc) or (IId) to produce a compound of formula (IIc″) or (IId″) respectively: 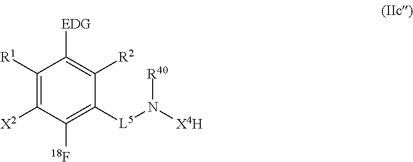
wherein EDG, R¹, R², L⁵ and R⁴⁰ are as defined in claim 1;
wherein X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀-alkylamino, di(C₁₋₁₀)alkylamino, acyl, amido, acylamido, halo, cyano and a group of formula (X), formula (X2), formula (Y), formula (Z1) or formula (Z2) as defined in claim 1; and
wherein X⁴ is said bidentate cleavable surrogate group which is bonded (a) to X¹ and (b) to H; 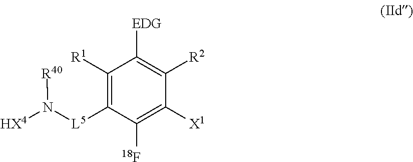
wherein EDG, R¹, R², L⁵ and R⁴⁰ are as defined in claim 1;
wherein X¹ is selected from H, unsubstituted or substituted -L⁵-N(R⁴⁰)H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2), as defined in claim 1; and
wherein X⁴ is said bidentate cleavable surrogate group which is bonded (a) to X² and (b) to H.

25. A process according to claim 1 wherein R³ in the compound of formula (I) is said bidentate cleavable surrogate group, X⁴, and wherein either:
(a) X¹ is a said group of formula (Z1) which is substituted with said bidentate cleavable surrogate group, X⁴, to form a group of formula (Z12) 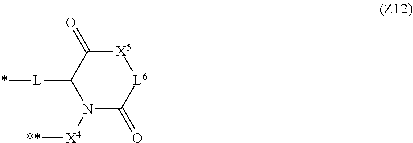
wherein * is the point of attachment of X¹ to the ring carbon atom meta to EDG or EDG′ and ** is the point of attachment of X⁴ to the ring carbon atom para to EDG or EDG′; or
(b) X² is a said group of formula (Z1) which is substituted with said bidentate cleavable surrogate group, X⁴, to form a said group of formula (Z12) 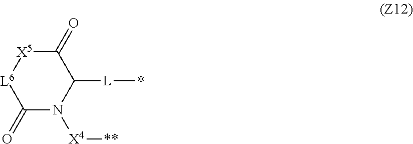
wherein * is the point of attachment of X² to the ring carbon atom meta to EDG or EDG′ and ** is the point of attachment of X⁴ to the ring carbon atom para to EDG or EDG′;
and the process further comprises rearomatisation of the compound of formula (IIc) or (IId) to produce a compound of formula (IIc′″) or (IId′″) respectively: 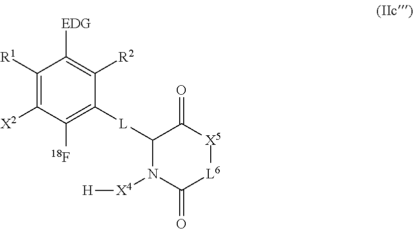
wherein EDG, R¹, R², L, X⁵ and L⁶ are as defined in claim 1;
wherein X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2) as defined in claim 1; and
wherein X⁴ is said bidentate cleavable surrogate group which is bonded (a) to X¹ and (b) to H; 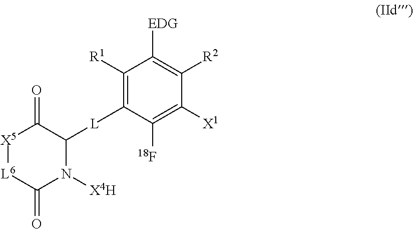
wherein EDG, R¹, R², L, X⁵ and L⁶ are as defined in claim 1;
wherein X¹ is selected from H, unsubstituted or substituted -L⁵-N(R⁴⁰)H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2), as defined in claim 1; and
wherein X⁴ is said bidentate cleavable surrogate group which is bonded (a) to X² and (b) to H.

26. A process according to claim 1 wherein R³ in the compound of formula (I) is said bidentate cleavable surrogate group X⁴ and the process further comprises:
(i) rearomatisation of said compound of formula (IIc) or (IId), comprising cleavage of X⁴ from the ring carbon atom para to EDG′ in said compound; and (ii) cleavage of X⁴ from the group X¹ or X² to which X⁴ is bonded;
thereby producing a compound of formula (II): 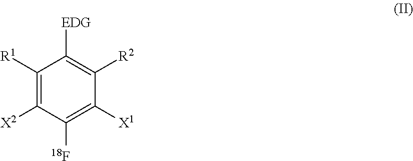
wherein EDG, R¹ and R² are as defined in claim 1; and
one of X¹ and X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, unsubstituted or substituted C₁₋₂₀ alkoxy, amino, unsubstituted or substituted C₁₋₁₀ alkylamino, unsubstituted or substituted di(C₁₋₁₀)alkylamino, unsubstituted or substituted acyl, unsubstituted or substituted amido, unsubstituted or substituted acylamido, halo, cyano and a group of formula (X), formula (X2), formula (Y), formula (Z1) or formula (Z2) as defined in claim 1; and
the other of X¹ and X² is selected from unsubstituted or substituted C₁₋₂₀ alkyl, unsubstituted or substituted -L⁵-N(R⁴⁰)H, unsubstituted or substituted C₃₋₂₀ cycloalkyl, unsubstituted or substituted aryl, unsubstituted or substituted heteroaryl, unsubstituted or substituted C₃₋₁₀ heterocyclyl, unsubstituted or substituted C₁₋₂₀ alkoxy, unsubstituted or substituted C₁₋₁₀ alkylamino, unsubstituted or substituted di(C₁₋₁₀)alkylamino, unsubstituted or substituted acyl, unsubstituted or substituted amido, unsubstituted or substituted acylamido, and a group of formula (X), formula (X2), formula (Y), formula (Z1) or formula (Z2).

27. A process according to claim 1 wherein R³ in the compound of formula (I) is said bidentate cleavable surrogate group, X⁴, and wherein either:
(a) X¹ is a said group of formula -L⁵-N(R⁴⁰)H which is substituted with said bidentate cleavable surrogate group, X⁴, to form a group of formula *-L⁵-N(R⁴⁰)—X⁴-**, wherein * is the point of attachment of X¹ to the ring carbon atom meta to EDG or EDG′ and ** is the point of attachment of X⁴ to the ring carbon atom para to EDG or EDG′; or
(b) X² is a said group of formula -L⁵-N(R⁴⁰)H which is substituted with said bidentate cleavable surrogate group, X⁴, to form a group of formula *-L⁵-N(R⁴⁰)—X⁴-**, wherein * is the point of attachment of X² to the ring carbon atom meta to EDG or EDG′ and ** is the point of attachment of X⁴ to the ring carbon atom para to EDG or EDG′;
and the process further comprises:
(i) rearomatisation of said compound of formula (IIc) or (IId), comprising cleavage of X⁴ from the ring carbon atom para to EDG′ in said compound; and
(ii) cleavage of X⁴ from the group X¹ or X² to which X⁴ is bonded; thereby producing a compound of formula (IIc″″) or (IId″″) respectively: 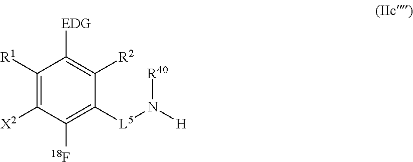
wherein EDG, R¹, R², L⁵ and R⁴⁰ are as defined in claim 1; and
X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2) as defined in claim 1; 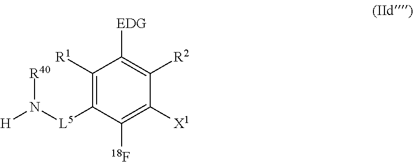
wherein EDG, R¹, R², L⁵ and R⁴⁰ are as defined in claim 1; and
X¹ is selected from H, unsubstituted or substituted -L⁵-N(R⁴⁰)H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2), as defined in claim 1.

28. A process according to claim 27 which further comprises a deprotection step comprising substituting H for said amino protecting group R⁴⁰, thereby converting the group —NHR⁴⁰ in the compound of formula (IIc″″) or (IId″″) into a —NH₂ group.

29. A process according to claim 1 wherein R³ in the compound of formula (I) is said bidentate cleavable surrogate group, X⁴, and wherein either:
(a) X¹ is a said group of formula (Z1) which is substituted with said bidentate cleavable surrogate group, X⁴, to form a group of formula (Z12) 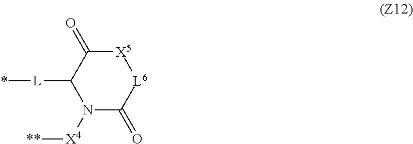
wherein * is the point of attachment of X¹ to the ring carbon atom meta to EDG or EDG′ and ** is the point of attachment of X⁴ to the ring carbon atom para to EDG or EDG′; or
(b) X² is a said group of formula (Z1) which is substituted with said bidentate cleavable surrogate group, X⁴, to form a said group of formula (Z12) 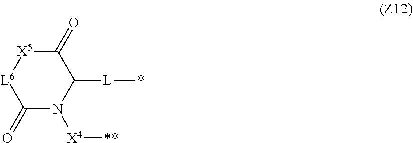
wherein * is the point of attachment of X² to the ring carbon atom meta to EDG or EDG′ and ** is the point of attachment of X⁴ to the ring carbon atom para to EDG or EDG′;
and the process further comprises
(i) rearomatisation of said compound of formula (IIc) or (IId), comprising cleavage of X⁴ from the ring carbon atom para to EDG′ in said compound; and
(ii) cleavage of X⁴ from the group X¹ or X² to which X⁴ is bonded;
thereby producing a compound of formula (IIc′″″) or (IId′″″) respectively: 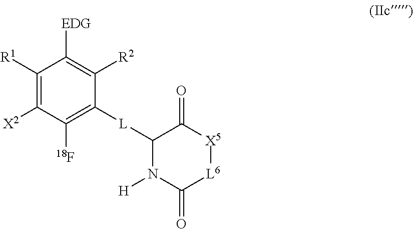
wherein EDG, R¹, R², L, X⁵ and L⁶ are as defined in claim 1; and
wherein X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2) as defined in claim 1; 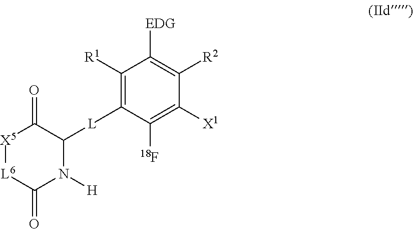
wherein EDG, R, R², L, X⁵ and L⁶ are as defined in claim 1; and
wherein X¹ is selected from H, unsubstituted or substituted -L⁵-N(R⁴⁰)H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2), as defined in claim 1.

30. A process according to claim 29 which further comprises a hydrolysis step, comprising hydrolysing the X⁵—C(O) bond and the N(H)—C(O) bond in the compound of formula (IIc′″″) or (IId′″″) in order to cleave the X⁵-L⁶-C(O) moiety from the compound, thereby producing a compound of formula (IIc″″″) or (IId″″″) respectively: 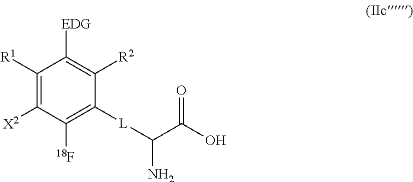
wherein EDG, R¹, R² and L are as defined in claim 1; and
X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2) as defined in claim 1; 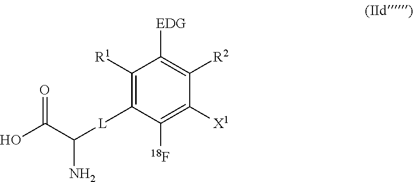
wherein EDG, R¹, R² and L are as defined in claim 1; and
wherein X¹ is selected from H, unsubstituted or substituted -L⁵-N(R⁴⁰)H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2), as defined in claim 1.

31. A process according to claim 1 wherein R³ in the compound of formula (I) is said bidentate cleavable surrogate group, X⁴, and wherein either:
(a) X¹ is a said group of formula (Z1) which is substituted with said bidentate cleavable surrogate group, X⁴, to form a group of formula (Z12) 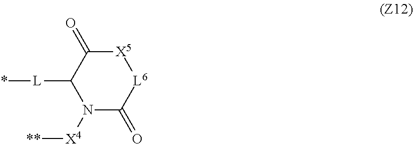
wherein * is the point of attachment of X¹ to the ring carbon atom meta to EDG or EDG′ and ** is the point of attachment of X⁴ to the ring carbon atom para to EDG or EDG′; or
(b) X² is a said group of formula (Z1) which is substituted with said bidentate cleavable surrogate group, X⁴, to form a said group of formula (Z12) 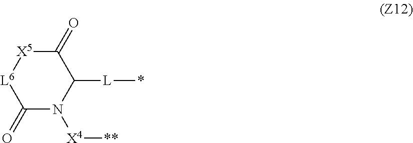
wherein * is the point of attachment of X² to the ring carbon atom meta to EDG or EDG′ and ** is the point of attachment of X⁴ to the ring carbon atom para to EDG or EDG′;
and the process further comprises
(i) rearomatisation of said compound of formula (IIc) or (IId), comprising cleavage of X⁴ from the ring carbon atom para to EDG′ in said compound;
(ii) cleavage of X⁴ from the group X¹ or X² to which X⁴ is bonded; and
(iii) cleaving the X⁵-L⁶-C(O) moiety from the group X¹ or X² to which X⁴ is bonded, thereby producing a compound of formula (IIc″″″) or (IId″″″) respectively: 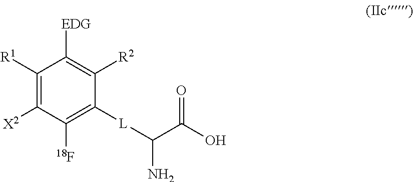
wherein EDG, R¹, R² and L are as defined in claim 1; and
X² is selected from H, unsubstituted or substituted C₁₋₂₀ alkyl, unsubstituted or substituted -L⁵-N(R⁴⁰)H, unsubstituted or substituted C₃₋₂₀ cycloalkyl, C₁₋₂ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀-alkylamino, di(C₁₋₁₀)alkylamino, acyl, amido, acylamido, halo, cyano and a group of formula (X), formula (X2), formula (Y), formula (Z1) or formula (Z2) as defined in claim 1; 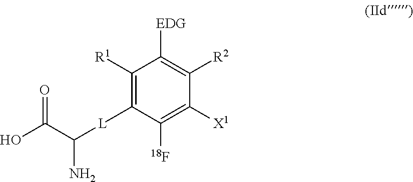
wherein EDG, R, R and L are as defined in claim 1; and
wherein X¹ is selected from H, unsubstituted or substituted -L⁵-N(R⁴⁰)H, unsubstituted or substituted C₁₋₂₀ alkyl, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2), as defined in claim 1.

32.-33. (canceled)

34. A process according to claim 23 wherein said rearomatisation is performed in situ.

35. A process according to claim 23 wherein said rearomatisation comprises the addition of a reagent which effects cleavage of X⁴ from the carbon atom of the ring which is para to EDG′ in the compound of formula (IIc) or formula (IId), wherein said reagent is an acid, base or oxidising agent.

36.-45. (canceled)

46. A process according to claim 1 wherein:
(a) EDG is —NHR⁵ and the process further comprises a deprotection step comprising substituting H for R⁵, thereby producing a compound wherein EDG is —NH₂; or
(b) EDG is —NHR⁵ or —NR⁵⁵R⁵ and the process further comprises a deprotection step comprising substituting H for R⁵ in the compound of formula (II), and, when R⁵⁵ is present, substituting H for R⁵⁵ in the compound of formula (II), thereby producing a compound of formula (IIb): 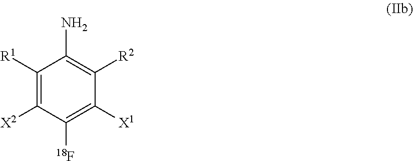
wherein
R¹ and R², which are the same or different, are independently selected from H, unsubstituted or substituted C₁₋₂₀ alkyl, unsubstituted or substituted C₃₋₁₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, acyl, amido, acylamido, halo, cyano, —OR¹⁰ and —NR¹¹R¹¹¹, wherein R¹⁰, R¹¹ and R¹¹¹ are as defined in claim 1; and
X¹ and X², which are the same or different, are independently selected from H, unsubstituted or substituted C₁₋₂₀ alkyl, unsubstituted or substituted -L⁵-N(R⁴⁰)H as defined in claim 1, unsubstituted or substituted C₃₋₂₀ cycloalkyl, C₁₋₂₀ perfluoroalkyl, unsubstituted or substituted aryl, perfluoroaryl, unsubstituted or substituted heteroaryl, unsubstituted or substituted C₃₋₁₀ heterocyclyl, hydroxyl, C₁₋₂₀ alkoxy, amino, C₁₋₁₀ alkylamino, di(C₁₋₁₀)alkylamino, acyl, amido, acylamido, halo, cyano and a group of formula (X), formula (X2), formula (Y), formula (Z1) or formula (Z2), as defined in claim 1,
provided that X² and R¹ may together form a bidentate group such that R¹, X² and the ring carbon atoms to which R¹ and X² are bonded together form an unsubstituted or substituted fused aryl, heteroaryl, C₅₋₈ carbocyclic or C₅₋₈ heterocyclic ring;
and provided that X¹ and R² may together form a bidentate group such that R², X¹ and the ring carbon atoms to which R² and X¹ are bonded together form an unsubstituted or substituted fused aryl, heteroaryl, C₅₋₈ carbocyclic or C₅₋₈ heterocyclic ring.

47. A process according to claim 46 wherein said deprotection step is performed in situ.

48.-51. (canceled)

52. A process according to claim 1 wherein the step of treating the compound of formula (I) with [¹⁸F]fluoride comprises treating the compound of formula (I) with a compound comprising ¹⁸F⁻ and a counter cation, wherein the counter cation is a quaternary ammonium cation, an alkali metal or H⁺.

53.-62. (canceled)

63. A process according to claim 1 wherein the oxidant is a hypervalent iodonium (III) reagent or a metal oxide.

64.-66. (canceled)

67. A process according to claim 1 wherein the step of treating the compound of formula (I) with [¹⁸F]fluoride is performed in the presence of an additive, wherein the additive is an acid or a crown ether.

68.-74. (canceled)

75. A process according to claim 1 wherein said step of treating said compound of formula (I) with said [¹⁸F]fluoride in the presence of said oxidant is performed in a microfluidic reactor.

76. A process according to claim 75 wherein said step of treating said compound of formula (I) with said [¹⁸F]fluoride in the presence of said oxidant comprises contacting a first solution comprising said compound of formula (I) and said [¹⁸F]fluoride with a second solution comprising said oxidant, in said microfluidic reactor.

77. A process according to claim 76 wherein said second solution further comprises an additive selected from the group consisting of acids and crown ethers.

78.-93. (canceled)

94. A process according to claim 1 wherein at least one of R¹ and R² is —OR¹⁰, wherein R¹⁰ is said hydroxyl protecting group, and the process further comprises a deprotection step, performed after said step of treating said compound of formula (I) with said [¹⁸F]fluoride, said deprotection step comprising substituting H for R¹⁰ in said group —OR¹⁰, thereby converting said group —OR¹⁰ into an —OH group.

95. A process according to claim 1, wherein EDG is OH or OR⁴, provided that when EDG is OR⁴, the process further comprises a deprotection step, performed after said step of treating said compound of formula (I) with said [¹⁸F]fluoride, said deprotection step comprising substituting H for R⁴ in said group —OR⁴, thereby converting said group —OR⁴ into an —OH group.

96.-107. (canceled)

108. A process according to claim 1 wherein at least one of X¹ and X² is a group of formula (X) in which at least one of R²² and R²³ is a said amino protecting group, and the process further comprises a deprotection step, performed after said step of treating said compound of formula (I) with said [¹⁸F]fluoride, said deprotection step comprising substituting H for said amino protecting group or groups, thereby converting the group —NR²²R²³ in the group of formula (X) into an —NH₂ group; and/or
at least one of X¹ and X² is a group of formula (X) in which R²⁴ is a said carboxyl protecting group, and the process further comprises a deprotection step, performed after said step of treating said compound of formula (I) with said [¹⁸F]fluoride, said deprotection step comprising substituting H for said carboxyl protecting group, thereby converting the group —COOR²⁴ in the group of formula (X) into a —COOH group,
wherein said deprotection step or steps result in the conversion of said group of formula (X) into a group of formula (Xa) or (Xb) 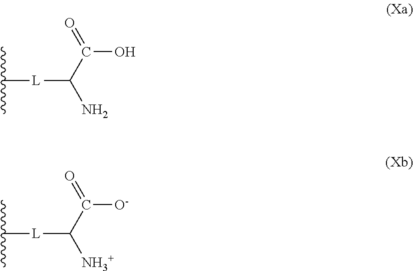
wherein L is unsubstituted or substituted C₁₋₄ alkylene.

109.-113. (canceled)

114. A process according to claim 1 wherein at least one of X¹ and X² is a group of formula (Y), and the process further comprises a deprotection step, performed after said step of treating said compound of formula (I) with said [¹⁸F]fluoride, said deprotection step comprising converting said group of formula (Y) into a group of formula (Xa) or (Xb) 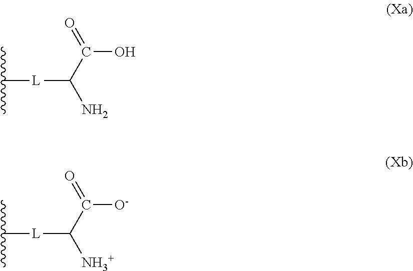
wherein L is unsubstituted or substituted C₁₋₄ alkylene.

115. (canceled)
